(19) United States
(12) Patent Application Publication
(10) Pub. No.: US 2026/0297198 A1
(43) Pub. Date: Oct. 1, 2026

(54) COMPOSITIONS AND METHODS INVOLVING INTEGRIN ALPHA3BETA1

(71) Applicants: 149 BIO, LLC, Miami, FL (US); Rush University Medical Center, Chicago, IL (US)

(72) Inventors: Santiago Balza Pineda, Miami, FL (US); Darlah Lopez-Rodriguez, Miami, FL (US); Carl J. Abrahams, Miami, FL (US); Antonio J. Barbosa, Miami, FL (US); Vineet Gupta, Miami, FL (US)

(21) Appl. No.: 19/124,718

(22) PCT Filed: Oct. 27, 2023
(86) PCT No.: PCT/US2023/078109

§ 371 Date: Jul. 21, 2025

Related U.S. Application Data
(60) Provisional application No. 63/420,964, filed on Oct. 31, 2022.

(51) Int. Cl. C07K 16/28; G01N 33/68

(52) U.S. Cl. CPC C07K 16/2842; G01N 33/6854; C07K 2317/24; C07K 2317/31; C07K 2317/622; G01N 2333/7055

(57) ABSTRACT

Compositions and methods comprising antibodies that bind to integrin α3β1 are provided.

### CROSS-REFERENCE TO RELATED APPLICATION

This application claims priority to U.S. Provisional Application No. 63/420,964, filed Oct. 31, 2022, the disclosure of which is hereby incorporated by reference in its entirety for all purposes.

### REFERENCE TO A SEQUENCE LISTING

The instant application contains a Sequence Listing which has been submitted electronically in XML file format and is hereby incorporated by reference in its entirety. Said XML copy, created on Oct. 27, 2025, is named 107889-1494591-001610US_SL.xml and is 84,428 bytes in size.

### BACKGROUND

Integrin α3β1 is a key integrin on the surface of cells, including podocytes, which are cells in Bowman's capsule in the kidneys that wrap around capillaries of the glomerulus. Integrin α3β1 is essential for podocyte attachment to the outside of blood vessels to form a healthy glomerulus in the kidney. Allosteric agonist antibodies to integrin α3β1 can enhance integrin-dependent ligand binding and cell adhesion.

### BRIEF SUMMARY

In one aspect, the disclosure features an isolated antibody that binds to integrin α3β1 or a portion thereof, comprising:
(1) a heavy chain complementarity-determining region 1 (CDR H1) comprising the sequence of X₁X₂SGX₃TFX₄X₅YX₆X₇X₈ (SEQ ID NO:38), wherein X₁ is A or K; X₂ is A or T; X₃ is F, G, or F; X₄ is S or T; X₅ is S or N; X₆ is G, S, or A; X₇ is M or I; and X₈ is H, N, or S;
(2) a CDR H2 comprising a sequence having up to two amino acid substitutions relative to the sequence of GISGSADTTY (SEQ ID NO:6), SISSSSSYIY (SEQ ID NO:9), or GIIPIFGTAN (SEQ ID NO:10), or the sequence of WISAX₁NGNX₂N (SEQ ID NO:39), wherein X₁ is Y or N; and X₂ is T or S;
(3) a CDR H3 comprising a sequence having up to two amino acid substitutions relative to the sequence of VRDDIQLRD (SEQ ID NO:11) or AREFPGWYFDY (SEQ ID NO:13), or a sequence having up to four amino acid substitutions relative to the sequence of

(SEQ ID NO: 12)
ARDYSGSWYPSNGPALDY,
(SEQ ID NO: 14)
AREYYDFWSGYPSGYAFDI,
or
(SEQ ID NO: 15)
ARGVPSGSGYYLGLDY;

(4) a light chain complementarity-determining region 1 (CDR L1) comprising the sequence of X₁ASQX₂ISX₃YLN (SEQ ID NO:40), wherein X₁ is Q or A; X₂ is D or Y; and X₃ is N or S, or a sequence having up to three amino acid substitutions relative to the sequence of QGDSLRSYYAS (SEQ ID NO:23) or SGSSSNIGSNYVY (SEQ ID NO:24);
(5) a CDR L2 comprising a sequence having up to one amino acid substitution relative to the sequence of YDASNLET (SEQ ID NO:25), or the sequence of YX₁X₂NX₃RPS (SEQ ID NO:41), wherein X₁ is G or R; X₂ is K or N; and X₃ is N or Q; and
(6) a CDR L3 comprising the sequence of X₁QX₂YX₃X₄PXST (SEQ ID NO:42), wherein X₁ is L or Q; X₂ is D or S; X₃ is N, S, or R; X₄ is Y or T; and X₅ is L or P, or a sequence having up to two amino acid substitutions relative to the sequence of

(SEQ ID NO: 31)
NSRDSSGNHWV
or
(SEQ ID NO: 32)
AAWDDSLSGPV.

In some embodiments of this aspect, (1) the CDR H1 comprises a sequence of any one of AASGFTFSSYGMH (SEQ ID NO:1), KASGYTFTSYGIS (SEQ ID NO:2), KTSGFTFTNYGIS (SEQ ID NO:3), AASGFTFSSYSMN (SEQ ID NO:4), and KASGGTFSSYAIN (SEQ ID NO:5); (2) the CDR H2 comprises a sequence of any one of GISGSADTTY (SEQ ID NO:6), WISAYNGNTN (SEQ ID NO:7), WISANNGNSN (SEQ ID NO:8), SISSSSSYIY (SEQ ID NO:9), and GIIPIFGTAN (SEQ ID NO:10); (3) the CDR H3 comprises a sequence of any one of VRDDIQLRD (SEQ ID NO:11), ARDYSGSWYPSNGPALDY (SEQ ID NO:12), AREFPGWYFDY (SEQ ID NO:13), AREYYDFWSGYPSGYAFDI (SEQ ID NO:14), and ARGVPSGSGYYLGLDY (SEQ ID NO:15); (4) the CDR L1 comprises the sequence of any one of QASQDISNYLN (SEQ ID NO:21), RASQYISSYLN (SEQ ID NO:22), QGDSLRSYYAS (SEQ ID NO:23), and SGSSSNIGSNYVY (SEQ ID NO:24); (5) the CDR L2 comprises a sequence of any one of YDASNLET (SEQ ID NO:25), YGKNNRPS (SEQ ID NO:26), and YRNNQRPS (SEQ ID NO:27); and (6) the CDR L3 comprises a sequence of any one of LQDYNYPLT (SEQ ID NO:28), LODYSYPLT (SEQ ID NO:29), QQSYRTPPT (SEQ ID NO:30), NSRDSSGNHWV (SEQ ID NO:31), and AAWDDSLSGPV (SEQ ID NO:32).

In some embodiments, the CDR H1 comprises the sequence of SEQ ID NO:1; the CDR H2 comprises the sequence of SEQ ID NO:6; and the CDR H3 comprises the sequence of SEQ ID NO:11.

In some embodiments, the CDR H1 comprises the sequence of SEQ ID NO:2; the CDR H2 comprises the sequence of SEQ ID NO:7; and the CDR H3 comprises the sequence of SEQ ID NO:12.

In some embodiments, the CDR H1 comprises the sequence of SEQ ID NO:3; the CDR H2 comprises the sequence of SEQ ID NO:8; and the CDR H3 comprises the sequence of SEQ ID NO:13.

In some embodiments, the CDR H1 comprises the sequence of SEQ ID NO:4; the CDR H2 comprises the sequence of SEQ ID NO:9; and the CDR H3 comprises the sequence of SEQ ID NO:14.

In some embodiments, the CDR H1 comprises the sequence of SEQ ID NO:5; the CDR H2 comprises the sequence of SEQ ID NO:10; and the CDR H3 comprises the sequence of SEQ ID NO:15.

In some embodiments, the CDR L1 comprises the sequence of SEQ ID NO:21; the CDR L2 comprises the sequence of SEQ ID NO:25; and the CDR L3 comprises the sequence of SEQ ID NO:28.

In some embodiments, the CDR L1 comprises the sequence of SEQ ID NO:22; the CDR L2 comprises the sequence of SEQ ID NO:25; and the CDR L3 comprises the sequence of SEQ ID NO:29.

In some embodiments, the CDR L1 comprises the sequence of SEQ ID NO:21; the CDR L2 comprises the sequence of SEQ ID NO:25; and the CDR L3 comprises the sequence of SEQ ID NO:30.

In some embodiments, the CDR L1 comprises the sequence of SEQ ID NO:23; the CDR L2 comprises the sequence of SEQ ID NO:26; and the CDR L3 comprises the sequence of SEQ ID NO:31.

In some embodiments, the CDR L1 comprises the sequence of SEQ ID NO:24; the CDR L2 comprises the sequence of SEQ ID NO:27; and the CDR L3 comprises the sequence of SEQ ID NO:32.

In some embodiments, the antibody comprises a heavy chain variable region having at least 90% identity to a sequence of any one of SEQ ID NOS: 16-20. In some embodiments, the antibody comprises a light chain variable region having at least 90% identity to a sequence of any one of SEQ ID NOS:33-37.

In some embodiments, the antibody comprises a HCDR1 having the sequence of SEQ ID NO:1, a HCDR2 having the sequence of SEQ ID NO:6, a HCDR3 having the sequence of SEQ ID NO:11, a LCDR1 having the sequence of SEQ ID NO:21, a LCDR2 having the sequence of SEQ ID NO:25, and a LCDR3 having the sequence of SEQ ID NO:28. In some embodiments, the antibody comprises a heavy chain variable region having at least 90% identity to the sequence of SEQ ID NO:16. In some embodiments, the antibody comprises a light chain variable region having at least 90% identity to the sequence of SEQ ID NO:33.

In some embodiments, the antibody comprises a HCDR1 having the sequence of SEQ ID NO:2, a HCDR2 having the sequence of SEQ ID NO:7, a HCDR3 having the sequence of SEQ ID NO:12, a LCDR1 having the sequence of SEQ ID NO:22, a LCDR2 having the sequence of SEQ ID NO:25, and a LCDR3 having the sequence of SEQ ID NO:29. In some embodiments, the antibody comprises a heavy chain variable region having at least 90% identity to the sequence of SEQ ID NO:17. In some embodiments, the antibody comprises a light chain variable region having at least 90% identity to the sequence of SEQ ID NO:34.

In some embodiments, the antibody comprises a HCDR1 having the sequence of SEQ ID NO:3, a HCDR2 having the sequence of SEQ ID NO:8, a HCDR3 having the sequence of SEQ ID NO:13, a LCDR1 having the sequence of SEQ ID NO:21, a LCDR2 having the sequence of SEQ ID NO:25, and a LCDR3 having the sequence of SEQ ID NO:30. In some embodiments, the antibody comprises a heavy chain variable region having at least 90% identity to the sequence of SEQ ID NO:18. In some embodiments, the antibody comprises a light chain variable region having at least 90% identity to the sequence of SEQ ID NO:35.

In some embodiments, the antibody comprises a HCDR1 having the sequence of SEQ ID NO:4, a HCDR2 having the sequence of SEQ ID NO:9, a HCDR3 having the sequence of SEQ ID NO:14, a LCDR1 having the sequence of SEQ ID NO:23, a LCDR2 having the sequence of SEQ ID NO:26, and a LCDR3 having the sequence of SEQ ID NO:31. In some embodiments, the antibody comprises a heavy chain variable region having at least 90% identity to the sequence of SEQ ID NO:19. In some embodiments, the antibody comprises a light chain variable region having at least 90% identity to the sequence of SEQ ID NO:36.

In some embodiments, the antibody comprises a HCDR1 having the sequence of SEQ ID NO:5, a HCDR2 having the sequence of SEQ ID NO:10, a HCDR3 having the sequence of SEQ ID NO:15, a LCDR1 having the sequence of SEQ ID NO:24, a LCDR2 having the sequence of SEQ ID NO:27, and a LCDR3 having the sequence of SEQ ID NO:32. In some embodiments, the antibody comprises a heavy chain variable region having at least 90% identity to the sequence of SEQ ID NO:20. In some embodiments, the antibody comprises a light chain variable region having at least 90% identity to the sequence of SEQ ID NO:37.

In some embodiments, the antibody comprises an Fc polypeptide having at least 90% identity to a sequence of SEQ ID NO:43.

In some embodiments of the antibody described herein, the antibody binds to a cell expressing integrin α3β1 or a portion thereof. In certain embodiments, the cell is a podocyte, a T cell, a cancer cell, or a neutrophil.

In some embodiments, the antibody binds to the α3 portion of the integrin α3β1. In some embodiments, the antibody binds to a sequence within a thigh-genu region in the α3 portion. In particular embodiments, the antibody binds to the sequence of SEQ ID NO:44 or a sequence within the sequence of SEQ ID NO:44. In some embodiments, the antibody binds to a specific conformation of α3. In some embodiments, the antibody binds α3 and stabilizes it in a specific conformation.

In certain embodiments, the antibody is a monoclonal antibody. In some embodiments, the antibody is a humanized antibody. In some embodiments, the antibody is a full-length antibody, a Fab, a Fab′, a F(ab′)2, an Fv, or a single chain Fv (scFv) antibody. In some embodiments, the antibody is a bispecific antibody.

In another aspect, the disclosure also provides an isolated nucleic acid encoding the isolated antibody described herein.

In another aspect, the disclosure provides an expression vector comprising the nucleic acid that encodes the isolated antibody described herein.

In another aspect, the disclosure provides an isolated host cell comprising the vector described above.

In another aspect, the disclosure provides a pharmaceutical composition comprising the isolated antibody described herein and a pharmaceutically acceptable carrier.

In another aspect, the disclosure provides a method for treating a disease or condition associated with a loss of podocytes in a subject in need thereof, comprising administering to the subject an isolated antibody described herein. In some embodiments, the disease or condition is a kidney disease, an autoimmune disease, a cancer, or an inflammation. In some embodiments, the disease or condition is a transplant procedure.

In some embodiments of the method, the kidney disease is a glomerular disease, such as a nephritic disease, a nephrotic disease, Alport's syndrome, or Focal Segmental Glomerulosclerosis (FSGS).

In another aspect, the disclosure features a method for identifying antibodies that bind to an integrin α3β1 or a portion thereof, comprising:
1) removing antibodies that bind against the β1 chain of the integrin α3β1 in the presence or absence of a ligand mimetic peptide and/or an antibody;
2) from the remaining antibodies from step 1), selecting for antibodies that bind to the integrin α3β1 in the presence or absence of a β1 agonist antibody;
3) counter selecting for antibodies that bind to the integrin α3β1 against immobilized β1 agonist antibodies or a ligand mimetic peptide alone; and
4) repeating steps 1), 2), and 3) above to enrich for antibodies that are integrin α3 allosteric agonists in the presence of cell surface-expressed integrin α3β1.

In some embodiments of the method, the ligand mimetic peptide is LXY2. In some embodiments of the method, step 1) and/or 3) is performed using β1 containing integrin dimers that are not α3β1, such as α4β1 and α5β1. In some embodiments of the method, step 1) and/or 2) is performed using human K562 cells that primarily express human α5β1 integrin and not over-express α3β1.

In some embodiments of the method, step 2) and/or 3) is performed using human K562 cells that over-express α3β1. In some embodiments, step 1) and/or 2) and/or 3) are performed in the presence of agents that block the ligand binding site or domain of the integrin, such as antibodies and ligands.

In some embodiments, integrin α3β1 is stabilized in a specific conformation by pre-complexing it with activating or inhibitory agents, such as activating antibody 9EG7 or TS2/16. In some other embodiments, integrin α3β1 is stabilized in a specific conformation by pre-complexing with agents that selectively bind the beta-chain of the integrin dimer.

### BRIEF DESCRIPTION OF THE DRAWINGS

FIGS. 1A-1D: Binding characterization of integrin agonist antibodies by Direct Integrin ELISA. Bovine Serum Albumin (BSA), recombinant human integrin α3β1 ECD, recombinant human integrin α4β1 ECD, or recombinant mouse integrin α3β1 ECD were coated on the plate and incubated with human anti-α3 Abs or isotype (n of 8 per coated protein, n of 4 for BSA). Binding of (A) Ab74 A100, (B) Ab74 A101, (C) Ab74 A102, and (D) Ab74 A104, was detected by incubating and developing fluorometric substrate with anti-hlgG1 Ab HRP conjugate, then reading mean fluorescence intensity on a plate reader.

FIG. 2: Epitope mapping of integrin agonist antibodies by Direct Integrin ELISA. Recombinant integrin α3β1 domains or Bovine Serum Albumin (BSA) were coated on the plate and incubated with human anti-α3 Abs (red) or isotype (blue) (n of 4). Binding of Ab74 A101 was detected by incubating and developing fluorometric substrate with anti-hIgG1 Ab HRP conjugate, then reading mean fluorescence intensity on a plate reader.

FIGS. 3A-3D: Increased ligand binding by mouse integrin α3β1 expressing cells in the presence of agonist antibodies. α3β1-expressing K562 cells are incubated with α3β1 ligand mimetic LXY2-biotin conjugate and either an integrin agonist antibody or isotype antibody control. Cells are then stained by Streptavidin-fluorophore conjugate and cells are measured in the flow cytometer. (A) Ab74 A100, (B) Ab74 A101, (C) Ab74 A102, (D) Ab74 A104 demonstrate increased LXY2 binding when compared to isotype control alone.

FIGS. 4A-4E: Reduced cell migration in the presence of integrin agonist antibodies by Wound Healing Assay. α3β1-expressing SK-OV-3 cells are plated on ligand-coated wells and allowed to adhere for 16 hours at 37° C. (A) Scratch wound created on the cell layer using a sterile pipette tip before adding treatment. (B) Isotype antibody control and (C) blocking anti-α3 antibody do not reduce cell migration, allowing cells to close the wound. (D) Control anti-β1 agonist antibody and (E) anti-α3 agonist antibody Ab74 A101 reduce wound closure after 16 hours.

FIG. 5: Schematic illustration of domain swapped mammalian expression constructs.

FIGS. 6A-6C: Staining of podocytes with antibodies shows that novel anti-integrin α3 antibodies stain podocyte expressed integrin α3β1. Kidney sections from C57B/L6 wildtype mouse were immunofluorescently stained with various antibodies (5 μg/mL) and imaged using confocal microscopy. Representative images showing staining with either Ab74_A100 (A), 9EG7 (B), or human anti-mouse IgG1 isotype control antibody (C).

### DETAILED DESCRIPTION

### I. Introduction

The inventors have discovered new antibodies that bind to integrin α3β1, e.g., a sequence within the thigh-genu region of integrin α3β1. Such antibodies act as agonists to integrin α3β1 and can enhance integrin-dependent functions, such as ligand binding and cell adhesion. In particular, given that integrin α3β1 is the key integrin on the surface of podocytes, the antibodies can be useful for treating diseases and/or conditions associated with a loss of podocytes, e.g., a kidney disease such as nephritic disease, a nephrotic disease, Alport's syndrome, or Focal Segmental Glomerulosclerosis (FSGS).

The inventors also found that the novel anti-integrin α3 allosteric antibodies induce intracellular signaling in the presence of external ligands. For example, level of phosphorylation focal adhesion kinase (pFAK) did not change when integrin α3-expressing cells were incubated with the novel anti-α3 integrin antibodies in the absence of integrin ligands. Co-incubation of cells with the novel antibodies and ligand laminin increased relative level of pFAK.

### II. Definitions

The term “antibody” as used herein includes antibody fragments that retain binding specificity. For example, there are a number of well characterized antibody fragments. Thus, for example, pepsin digests an antibody C-terminal to the disulfide linkages in the hinge region to produce F(ab)′2, a dimer of Fab which itself is a light chain joined to V_{H}-C_{H}1 by a disulfide bond. The F(ab)′2 may be reduced under mild conditions to break the disulfide linkage in the hinge region thereby converting the (Fab′)2 dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that fragments can be synthesized de novo either chemically or by utilizing recombinant DNA methodology. Thus, the term antibody, as used herein also includes antibody fragments either produced by the modification of whole antibodies or synthesized using recombinant DNA methodologies.

An antibody as described herein can consist of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. In some embodiments, the antibody is IgG (e.g., IgG1, IgG2, IgG3, IgG4), IgM, IgA, IgD, or IgE.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

In an antibody, substitution variants have at least one amino acid residue removed and a different residue inserted in its place. The sites of greatest interest for substitutional mutagenesis include the hypervariable regions, but framework alterations are also contemplated. Examples of conservative substitutions are described above.

Substantial modifications in the biological properties of the antibody are accomplished by selecting substitutions that differ significantly in their effect on maintaining (a) the structure of the polypeptide backbone in the area of the substitution, for example, as a β-sheet or helical conformation, (b) the charge or hydrophobicity of the molecule at the target site, or (c) the bulk of the side chain. Naturally occurring residues are divided into groups based on common side-chain properties:
(1) Non-polar: Norleucine, Met, Ala, Val, Leu, Ile;
(2) Polar without charge: Cys, Ser, Thr, Asn, Gln;
(3) Acidic (negatively charged): Asp, Glu;
(4) Basic (positively charged): Lys, Arg;
(5) Residues that influence chain orientation: Gly, Pro; and
(6) Aromatic: Trp, Tyr, Phe, His.
   Non-conservative substitutions are made by exchanging a member of one of these classes for another class.

One type of substitution that can be made is to change one or more cysteines in the antibody, which may be chemically reactive, to another residue, such as, without limitation, alanine or serine. For example, there can be a substitution of a non-canonical cysteine. The substitution can be made in a CDR or framework region of a variable domain or in the constant region of an antibody. In some embodiments, the cysteine is canonical (e.g., involved in disulfide bond formation). Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment.

Antibodies include V_{H}-V_{L} dimers, including single chain antibodies (antibodies that exist as a single polypeptide chain), such as single chain Fv antibodies (sFv or scFv) in which a variable heavy and a variable light region are joined together (directly or through a peptide linker) to form a continuous polypeptide. The single chain Fv antibody is a covalently linked V_{H}-V_{L} which may be expressed from a nucleic acid including V_{H}- and V_{L}-encoding sequences either joined directly or joined by a peptide-encoding linker (e.g., Huston, et al. Proc. Nat. Acad. Sci. USA, 85:5879-5883, 1988). While the V_{H} and V_{L} are connected to each as a single polypeptide chain, the V_{H} and V_{L} domains associate non-covalently. Alternatively, the antibody can be another fragment. Other fragments can also be generated, e.g., using recombinant techniques, as soluble proteins or as fragments obtained from display methods. Antibodies can also include diantibodies and miniantibodies. Antibodies of the disclosure also include heavy chain dimers, such as antibodies from camelids. In some embodiments an antibody is dimeric. In other embodiments, the antibody may be in a monomeric form that has an active isotype. In some embodiments the antibody is in a multivalent form, e.g., a trivalent or tetravalent form.

As used herein, the terms “variable region” and “variable domain” refer to the portions of the light and heavy chains of an antibody that include amino acid sequences of complementary determining regions (CDRs, e.g., HCDR1, HCDR2, HCR3, LCDR1, LCDR2, and LCDR3) and framework regions (FRs). The variable region for the heavy and light chains is commonly designated V_{H} and V_{L}, respectively. The variable region is included on Fab, F(ab′)₂, Fv and scFv antibody fragments described herein, and involved in specific antigen recognition.

As used herein, “complementarity-determining region (CDR)” refers to the three hypervariable regions in each chain that interrupt the four framework regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

The sequences of the framework regions of different light or heavy chains are relatively conserved within a species. The framework region of an antibody, that is the combined framework regions of the constituent light and heavy chains, serves to position and align the CDRs in three dimensional space.

The amino acid sequences of the CDRs and framework regions can be determined using various well known definitions in the art, e.g., Kabat, North method (see, e.g., North et al., J Mol Biol. 406(2):228-256, 2011), Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human V_{H} segments J. Mol. Biol. 227, 799-817; Al-Lazikani et al., J. Mol. Biol 1997, 273 (4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996).

As used herein, the term “allosteric agonist” refers to a molecule (e.g., an antibody) that binds to its target (e.g., integrin α3β1 or a portion thereof, a sequence within α3 portion of the integrin α3β1, a sequence of SEQ ID NO:44 or a portion thereof), at a site or area that is not the target's active site, to enhance, activate, or increase the target's response to the binding of its natural ligand.

As used herein, “chimeric antibody” refers to an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region, or portion thereof, having a different or altered antigen specificity; or with corresponding sequences from another species or from another antibody class or subclass.

As used herein, “humanized antibody” refers to an immunoglobulin molecule in CDRs from a donor antibody are grafted onto human framework sequences. Humanized antibodies may also comprise residues of donor origin in the framework sequences. The humanized antibody can also comprise at least a portion of a human immunoglobulin constant region. Humanized antibodies may also comprise residues which are found neither in the recipient antibody nor in the imported CDR or framework sequences. Humanization can be performed using methods known in the art (e.g., Jones et al., Nature 321:522-525; 1986; Riechmann et al., Nature 332:323-327, 1988; Verhoeyen et al., Science 239:1534-1536, 1988); Presta, Curr. Op. Struct. Biol. 2:593-596, 1992; U.S. Pat. No. 4,816,567), including techniques such as “superhumanizing” antibodies (Tan et al., J. Immunol. 169:1119, 2002) and “resurfacing” (e.g., Staelens et al., Mol. Immunol. 43:1243, 2006; and Roguska et al., Proc. Natl. Acad. Sci USA 91:969, 1994).

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The terms “antigen,” “immunogen,” “antibody target,” “target analyte,” and like terms are used herein to refer to a molecule, compound, or complex that is recognized by an antibody, i.e., can be specifically bound by the antibody. The term can refer to any molecule that can be specifically recognized by an antibody, e.g., a polypeptide, polynucleotide, carbohydrate, lipid, chemical moiety, or combinations thereof (e.g., phosphorylated or glycosylated polypeptides, etc.). One of skill will understand that the term does not indicate that the molecule is immunogenic in every context, but simply indicates that it can be targeted by an antibody.

Antibodies bind to an “epitope” on an antigen. The epitope is the localized site on the antigen that is recognized and bound by the antibody. Epitopes can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope). The same is true for other types of target molecules that form three-dimensional structures. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The terms “specific for,” “specifically binds,” and like terms refer to a molecule (e.g., antibody or antibody fragment) that binds to a target with at least 2-fold greater affinity than non-target compounds, e.g., at least any of 4-fold, 5-fold, 6-fold, 7-fold, 8-fold, 9-fold, 10-fold, 20-fold, 25-fold, 50-fold, or 100-fold greater affinity. For example, an antibody that specifically binds a target will typically bind the target with at least a 2-fold greater affinity than a non-target. Specificity can be determined using standard methods, e.g., solid-phase ELISA immunoassays (see, e.g., Harlow & Lane, Using Antibodies, A Laboratory Manual (1998) for a description of immunoassay formats and conditions that can be used to determine specific immunoreactivity).

The term “binds” with respect to an antibody target (e.g., antigen, analyte, immune complex), typically indicates that an antibody binds a majority of the antibody targets in a pure population (assuming appropriate molar ratios). For example, an antibody that binds a given antibody target typically binds to at least ⅔ of the antibody targets in a solution (e.g., at least any of 75, 80, 85, 90, 91, 92, 93, 94, 95, 96, 97, 98, 99, or 100%). One of skill will recognize that some variability will arise depending on the method and/or threshold of determining binding.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value or a range gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of benefit and/or side effects). Controls can be designed for in vitro applications. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST 2.0 sequence comparison algorithms with default parameters described below, or by manual alignment and visual inspection (see, e.g., NCBI web site ncbi.nlm.nih.gov/BLAST/or the like). Such sequences are then said to be “substantially identical.” As described below, the preferred algorithms can account for gaps and the like. Preferably, identity exists over a region that is at least about 25 amino acids or nucleotides in length, or more preferably over a region that is 50-100 or more amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art.

An algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the disclosure. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The term “nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

The terms “polypeptide,” “peptide” and “protein” are used interchangeably herein to refer to a polymer of amino acid residues. The terms encompass to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

### III. Antibodies that Bind Integrin α3β1 or a Portion Thereof

Antibodies (including antibody fragments) that specifically bind to integrin α3β1 or a portion thereof (e.g., a sequence within a thigh-genu region of integrin α3β1) are provided herein. Integrin α3β1 is an integrin heterodimer of α3 and β1 portions. Integrin α3β1 is highly expressed on the surface of kidney podocyte cells and is essential for podocyte attachment to the outside of blood vessels to form a healthy glomerulus in the kidney. This integrin is also expressed on other cells, such as T-cells (Park et al., Integrin α3 promotes TH17 cell polarization and extravasation during autoimmune neuroinflammation, Science Immunology, Vol 8 (88), 2023), cancer cells (Ke et al., Novel monoclonal antibody against integrin α3 shows therapeutic potential for ovarian cancer, Cancer Sci., 111 (10), p 3478, 2020) and neutrophils (Lerman et al., Sepsis lethality via exacerbated tissue infiltration and TLR-induced cytokine production by neutrophils is integrin α3β1-dependent, Blood, 2014 Dec. 4; 124(24):3515-23) and keratinocytes (Has et al, Integrin α3 mutations with kidney, lung, and skin disease. N Engl J Med 366:1508-1514, 2012), and can affect functions of cells that express this integrin. The antibodies described herein act as allosteric agonist antibodies to integrin α3β1 and can enhance integrin-dependent ligand binding and cell adhesion, thus, preventing podocyte cell loss in the urine and protecting from loss in kidney function. The antibodies can also reduce T-cell transmigration and infiltration to reduce autoimmune diseases, cancer cell migration to reduce tumor growth and metastases, and pro-inflammatory neutrophil activation and tissue recruitment.

In some embodiments, the anti-α3β1 antibody is isolated (e.g., separated from a component of its natural environment (e.g., an animal, a biological sample)). In some embodiments, the anti-α3β1 antibody is a humanized antibody, or an antigen binding fragment thereof. In some embodiments, the anti-α3β1 antibody is a derivative of a humanized antibody that binds α3β1 or a portion thereof. In some embodiments, the anti-α3β1 antibody binds α3β1 under laboratory conditions (e.g., binds α3β1 in vitro, binds α3β1 in a flow cytometry assay, binds α3β1 in an ELISA). In some embodiments, the anti-α3β1 antibody binds α3β1 under physiological conditions (e.g., binds α3β1 in a cell (e.g., a podocyte) in a subject).

In some embodiments, the α3 portion of the heterodimer integrin α3β1 has a sequence having at least 90% (e.g., 91%, 92%, 93%, 94%, 95%, 96%, 97%, 98%, 99%, or 100%) identity to the sequence of:

(SEQ ID NO: 45)
MGPGPSRAPRAPRLMLCALALMVAAGGCVVSAFNLDTRFLVVKEAGNPG
SLFGYSVALHRQTERQQRYLLLAGAPRELAVPDGYTNRTGAVYLCPLTA
HKDDCERMNITVKNDPGHHIIEDMWLGVTVASQGPAGRVLVCAHRYTQV
LWSGSEDQRRMVGKCYVRGNDLELDSSDDWQTYHNEMCNSNTDYLETGM
CQLGTSGGFTQNTVYFGAPGAYNWKGNSYMIQRKEWDLSEYSYKDPEDQ
GNLYIGYTMQVGSFILHPKNITIVTGAPRHRHMGAVFLLSQEAGGDLRR
RQVLEGSQVGAYFGSAIALADLNNDGWQDLLVGAPYYFERKEEVGGAIY
VFMNQAGTSFPAHPSLLLHGPSGSAFGLSVASIGDINQDGFQDIAVGAP
FEGLGKVYIYHSSSKGLLRQPQQVIHGEKLGLPGLATFGYSLSGQMDVD
ENFYPDLLVGSLSDHIVLLRARPVINIVHKTLVPRPAVLDPALCTATSC
VQVELCFAYNQSAGNPNYRRNITLAYTLEADRDRRPPRLRFAGSESAVF
HGFFSMPEMRCQKLELLLMDNLRDKLRPIIISMNYSLPLRMPDRPRLGL
RSLDAYPILNQAQALENHTEVQFQKECGPDNKCESNLQMRAAFVSEQQQ
KLSRLQYSRDVRKLLLSINVTNTRTSERSGEDAHEALLTLVVPPALLLS
SVRPPGACQANETIFCELGNPFKRNQRMELLIAFEVIGVTLHTRDLQVQ
LQLSTSSHQDNLWPMILTLLVDYTLQTSLSMVNHRLQSFFGGTVMGESG
MKTVEDVGSPLKYEFQVGPMGEGLVGLGTLVLGLEWPYEVSNGKWLLYP
TEITVHGNGSWPCRPPGDLINPLNLTLSDPGDRPSSPQRRRRQLDPGGG
QGPPPVTLAAAKKAKSETVLTCATGRAHCVWLECPIPDAPVVTNVTVKA
RVWNSTFIEDYRDFDRVRVNGWATLFLRTSIPTINMENKTTWFSVDIDS
ELVEELPAEIELWLVLVAVGAGLLLLGLIILLLWKCGFFKRARTRALYE
AKRQKAEMKSQPSETERLTDDY. The thigh-genu region is
in bold in SEQ ID NO: 45.

In some embodiments, the β1 portion of the heterodimer integrin α3β1 has a sequence having at least 90% (e.g., 91%, 92%, 93%, 94%, 95%, 96%, 97%, 98%, 99%, or 100%) identity to the sequence of:

(SEQ ID NO: 46)
MNLQPIFWIGLISSVCCVFAQTDENRCLKANAKSCGECIQAGPNCGWCTNSTFLQEG
MPTSARCDDLEALKKKGCPPDDIENPRGSKDIKKNKNVTNRSKGTAEKLKPEDITQI
QPQQLVLRLRSGEPQTFTLKFKRAEDYPIDLYYLMDLSYSMKDDLENVKSLGTDLM
NEMRRITSDFRIGFGSFVEKTVMPYISTTPAKLRNPCTSEQNCTSPFSYKNVLSLTNKG
EVFNELVGKQRISGNLDSPEGGFDAIMQVAVCGSLIGWRNVTRLLVFSTDAGFHFAG
DGKLGGIVLPNDGQCHLENNMYTMSHYYDYPSIAHLVQKLSENNIQTIFAVTEEFQP
VYKELKNLIPKSAVGTLSANSSNVIQLIIDAYNSLSSEVILENGKLSEGVTISYKSYCK
NGVNGTGENGRKCSNISIGDEVQFEISITSNKCPKKDSDSFKIRPLGFTEEVEVILQYIC
ECECQSEGIPESPKCHEGNGTFECGACRCNEGRVGRHCECSTDEVNSEDMDAYCRK
ENSSEICSNNGECVCGQCVCRKRDNTNEIYSGKFCECDNFNCDRSNGLICGGNGVCK
CRVCECNPNYTGSACDCSLDTSTCEASNGQICNGRGICECGVCKCTDPKFQGQTCEM
CQTCLGVCAEHKECVQCRAFNKGEKKDTCTQECSYFNITKVESRDKLPQPVQPDPVS
HCKEKDVDDCWFYFTYSVNGNNEVMVHVVENPECPTGPDIIPIVAGVVAGIVLIGLA
LLLIWKLLMIIHDRREFAKFEKEKMNAKWDTGENPIYKSAVTTVVNPKYEGK.

In certain embodiments, the antibody binds to the α3 portion (e.g., SEQ ID NO:45) of the integrin α3β1. In some embodiments, the antibody binds to a sequence within a thigh-genu region in the α3 portion. In particular embodiments, the antibody binds to the sequence of SEQ ID NO:44 or a portion within the sequence of SEQ ID NO:44.

(SEQ ID NO: 44)
VINIVHKTLVPRPAVLDPALCTATSCVQVELCFAYNQSAGNPNYRRNITL
AYTLEADRDRRPPRLRFAGSESAVFHGFFSMPEMRCQKLELLLMDNLRDK
LRPIIISMNYSLPLRMPDRPRLGLRSLDAYPILNQAQALENHTEVQFQKE
C.

Generally, the anti-α3β1 antibodies provided herein comprise at least one immunoglobulin heavy chain variable region and at least one immunoglobulin light chain variable region. In some embodiments, an anti-α3β1 antibody described herein comprises two immunoglobulin heavy chain variable regions and two immunoglobulin light chain variable regions. Typically, each immunoglobulin heavy chain variable region of the anti-α3β1 antibody comprises first, second, and third heavy chain complementarity determining regions (CDRs; HCDR1, HCDR2, and HCDR3), and each immunoglobulin light chain variable region of the anti-α3β1 antibody comprises first, second, and third light chain CDRs (LCDR1, LCDR2, and LCDR3).

In some embodiments, the antibodies are antibody fragments such as Fab, F(ab′)₂, Fv or scFv. The antibody fragments can be generated using any means known in the art including, chemical digestion (e.g., papain or pepsin) and recombinant methods. Methods for isolating and preparing recombinant nucleic acids are known to those skilled in the art (see, Sambrook et al., Molecular Cloning. A Laboratory Manual (2d ed. 1989); Ausubel et al., Current Protocols in Molecular Biology (1995)). The antibodies can be expressed in a variety of host cells, including E. coli, other bacterial hosts, yeast, and various higher eukaryotic cells such as the COS, CHO, and HeLa cells lines and myeloma cell lines.

In some embodiments, antibodies of the disclosure can comprise sequences of a heavy chain complementary determining region 1 (HCDR1), an HCDR2, an HCDR3, a light chain complementary determining region 1 (LCDR1), a LCDR2, a LCDR3, a heavy chain variable region (V_{H}), and/or a light chain variable region (V_{L}) as described in Table 1.

TABLE 1

| AB | HCDR1 | HCDR2 | HCDR3 | V_{H} |
|---|---|---|---|---|
| A100 | AASGFTFS | GISGSA | VRDDI | EVQLLESGGGVVQPGRSLRLSCAAS |
| | SYGMH | DTTY | QLRD | GFTFSSYGMHWVRQAPGKGLEWLS |
| | (SEQ ID | (SEQ ID | (SEQ ID | GISGSADTTYYADSVKGRFTISRDN |
| | NO: 1) | NO: 6) | NO: 11) | SKNTLYLQMTSLRAEDTAVYYCVR |
| | | | | DDIQLRDWGQGTLVTVSS (SEQ ID |
| | | | | NO: 16) |
| A101 | KASGYTF | WISAY | ARDYS | QVQLVQSGAEVKKPGASVKVSCKA |
| | TSYGIS | NGNTN | GSWYP | SGYTFTSYGISWVRQAPGQGLEWM |
| | (SEQ ID | (SEQ ID | SNGPA | GWISAYNGNTNYAQKLQGRVTMT |
| | NO: 2) | NO: 7) | LDY | TDTSTSTAYMELRSLRSDDTAVYY |
| | | | (SEQ ID | CARDYSGSWYPSNGPALDYWGQG |
| | | | NO: 12) | TMVTVSS (SEQ ID NO: 17) |
| A102 | KTSGFTFT | WISAN | AREFP | EVQLVQSGAEVKKPGASVKVSCKT |
| | NYGIS | NGNSN | GWYF | SGFTFTNYGISWVRQAPGQGLEWM |
| | (SEQ ID | (SEQ ID | DY | GWISANNGNSNYAQDHQGRVTMT |
| | NO: 3) | NO: 8) | (SEQ ID | TDTSTSTAYMELRSLRSDDTAVYY |
| | | | NO: 13) | CAREFPGWYFDYWGQGTLVTVSS |
| | | | | (SEQ ID NO: 18) |
| A103 | AASGFTFS | SISSSS | AREYY | EVQLVESGGGLVQPGGSLRLSCAAS |
| | SYSMN | SYIY | DFWSG | GFTFSSYSMNWVRQAPGKGLEWVS |
| | (SEQ ID | (SEQ ID | YPSGY | SISSSSSYIYYADSVKGRFTISRDNS |
| | NO: 4) | NO: 9) | AFDI | KNTVYLQMNSLRAEDTAVYYCAR |
| | | | (SEQ ID | EYYDFWSGYPSGYAFDIWGQGTLV |
| | | | NO: 14) | TVSS (SEQ ID NO: 19) |
| A104 | KASGGTF | GIIPIFG | SGSGY | QVQLVQSGAEVKKPGSSVKVSCKA |
| | SSYAIN | TAN | ARGVP | SGGTFSSYAINWVRQAPGQGLEWM |
| | (SEQ ID | (SEQ ID | YLGLD | GGIIPIFGTANYAQKFQGRVTITADK |
| | NO: 5) | NO: 10) | Y (SEQ | STSTAYMELSSLRSEDTAVYYCAR |
| | | | ID | GVPSGSGYYLGLDYWGQGTMVTV |
| | | | NO: 15) | SS (SEQ ID NO: 20) |
| AB | LCDR1 | LCDR2 | LCDR3 | V_{L} |
| A100 | QASQD | LET | LQDYN | DIQMTQSPSSLSASVGDRVTIT |
| | ISNYLN | (SEQ ID | YPLT | CQASQDISNYLNWYQQKPGK |
| | (SEQ ID | YDASN | (SEQ ID | APKLLIYDASNLETGVPSRFSG |
| | NO: 21) | NO: 25) | NO: 28) | SGSGTDFALTISSLQPEDFATY |
| | | | | YCLQDYNYPLTFGGGTKVDIK |
| | | | | (SEQ ID NO: 33) |
| A101 | RASQYI | YDASN | LQDYS | DIQMTQSPSSLSASVGDRVTIT |
| | SSYLN | LET | YPLT | CRASQYISSYLNWYQQKPGKA |
| | (SEQ ID | (SEQ ID | (SEQ ID | PKLLIYDASNLETGVPSRFSGS |
| | NO: 22) | NO: 25) | NO: 29) | GSGTDFTFTISSLQPEDIATYYC |
| | | | | LQDYSYPLTFGGGIKVDIK |
| | | | | SEQ ID NO: 34) |
| A102 | QASQD | LET | QQSYR | DIQMTQSPSSLSASVGDRVTIT |
| | ISNYLN | YDASN | TPPT | CQASQDISNYLNWYQQKPGK |
| | (SEQ ID | (SEQ ID | (SEQ ID | APKLLIYDASNLETGVPSRFSG |
| | NO: 21) | NO: 25) | NO: 30) | SGSGTDFTFTISSLQPDDFATY |
| | | | | YCQQSYRTPPTFGQGTKLEIK |
| | | | | (SEQ ID NO: 35) |
| A103 | QGDSL | YGKN | NSRDS | QSALTQDPAVSVALGQTVRIT |
| | RSYYA | NRPS | SGNH | CQGDSLRSYYASWYQQKPGQ |
| | S (SEQ | (SEQ ID | WV | APVLVIYGKNNRPSGIPDRFSG |
| | ID | NO: 26) | (SEQ ID | SSSGNTASLTITGAQAEDEAD |
| | NO: 23) | | NO: 31) | YYCNSRDSSGNHWVFGGGTK |
| | | | | LTVL (SEQ ID NO: 36) |
| A104 | SGSSSN | YRNNQ | AAWD | QSELTQPPSASGAPGQRVTISC |
| | IGSNY | RPS | DSLSG | SGSSSNIGSNYVYWYQQLPGT |
| | VY | (SEQ ID | PV | APKLLIYRNNQRPSGVPDRFSG |
| | (SEQ ID | NO: 27) | (SEQ ID | SKSGTSASLAISGLRSEDEADY |
| | NO: 24) | | NO: 32) | YCAAWDDSLSGPVFSGGTKLT |
| | | | | VL (SEQ ID NO: 37) |

An isolated antibody that specifically binds to integrin α3β1 or a portion thereof (e.g., a sequence within a thigh-genu region of integrin α3β1) can comprise:
(1) a heavy chain complementarity-determining region 1 (HCDR1) comprising the sequence of X₁X₂SGX₃TFX₄X₅YX₆X₇X₈ (SEQ ID NO:38), wherein X₁ is A or K; X₂ is A or T; X₃ is F, G, or F; X₄ is S or T; X₅ is S or N; X₆ is G, S, or A; X₇ is M or I; and X₈ is H, N, or S;
(2) a HCDR2 comprising a sequence having up to two amino acid substitutions relative to the sequence of GISGSADTTY (SEQ ID NO:6), SISSSSSYIY (SEQ ID NO:9), or GIIPIFGTAN (SEQ ID NO:10), or the sequence of WISAX₁NGNX₂N (SEQ ID NO:39), wherein X₁ is Y or N; and X₂ is T or S;
(3) a HCDR3 comprising a sequence having up to two amino acid substitutions relative to the sequence of VRDDIQLRD (SEQ ID NO:11) or AREFPGWYFDY (SEQ ID NO:13), or a sequence having up to four amino acid substitutions relative to the sequence of

(SEQ ID NO: 12)
ARDYSGSWYPSNGPALDY,
(SEQ ID NO: 14)
AREYYDFWSGYPSGYAFDI,
or
(SEQ ID NO: 15)
ARGVPSGSGYYLGLDY;

(4) a light chain complementarity-determining region 1 (LCDR1) comprising the sequence of X₁ASQX₂ISX₃YLN (SEQ ID NO:40), wherein X₁ is Q or A; X₂ is D or Y; and X₃ is N or S, or a sequence having up to three amino acid substitutions relative to the sequence of QGDSLRSYYAS (SEQ ID NO:23) or SGSSSNIGSNYVY (SEQ ID NO:24);
(5) a LCDR2 comprising a sequence having up to one amino acid substitution relative to the sequence of YDASNLET (SEQ ID NO:25), or the sequence of YX₁X₂NX₃RPS (SEQ ID NO:41), wherein X₁ is G or R; X₂ is K or N; and X₃ is N or Q; and
(6) a LCDR3 comprising the sequence of X₁QX₂YX₃X₄PX₅T (SEQ ID NO:42), wherein X₁ is L or Q; X₂ is D or S; X₃ is N, S, or R; X₄ is Y or T; and X₅ is L or P, or a sequence having up to two amino acid substitutions relative to the sequence of

(SEQ ID NO: 31)
NSRDSSGNHWV
or
(SEQ ID NO: 32)
AAWDDSLSGPV.

In some embodiments, an antibody of the disclosure comprises: an HCDR1 having a sequence of any one of SEQ ID NOS: 1-5 or a variant thereof that has a sequence having one amino acid substitution relative to a sequence of any one of SEQ ID NOS: 1-5. In some embodiments, an antibody of the disclosure comprises: an HCDR2 having a sequence of any one of SEQ ID NOS: 6-10 or a variant thereof that has a sequence having one amino acid substitution relative to a sequence of any one of SEQ ID NOS: 6-10. In some embodiments, an antibody of the disclosure comprises: an HCDR3 having a sequence of any one of SEQ ID NOS: 11-15 or a variant thereof that has a sequence having one amino acid substitution relative to a sequence of any one of SEQ ID NOS: 11-15.

In some embodiments, an antibody of the disclosure comprises: a LCDR1 having a sequence of any one of SEQ ID NOS: 21-24 or a variant thereof that has a sequence having one amino acid substitution relative to a sequence of any one of SEQ ID NOS: 21-24. In some embodiments, an antibody of the disclosure comprises: a LCDR2 having a sequence of any one of SEQ ID NOS: 25-27 or a variant thereof that has a sequence having one substitution relative to a sequence of any one of SEQ ID NOS: 25-27. In some embodiments, an antibody of the disclosure comprises: a LCDR3 having a sequence of any one of SEQ ID NOS: 28-32 or a variant thereof that has a sequence having one amino acid substitution relative to the sequence of SEQ ID NO:28-32.

### HCDR1-3 and V_{H}

In some embodiments, an antibody of the disclosure can comprise an HCDR1 having the sequence of SEQ ID NO:1 or a variant thereof that has a sequence having one amino acid substitution relative to the sequence of SEQ ID NO:1, an HCDR2 having the sequence of SEQ ID NO:6 or a variant thereof that has a sequence having one amino acid substitution relative to the sequence of SEQ ID NO:6, and an HCDR3 having the sequence of SEQ ID NO:11 or a variant thereof that has a sequence having one amino acid substitution relative to the sequence of SEQ ID NO:11. In some embodiments, an antibody of the disclosure can comprise an HCDR1 having the sequence of SEQ ID NO:2 or a variant thereof that has a sequence having one amino acid substitution relative to the sequence of SEQ ID NO:2, an HCDR2 having the sequence of SEQ ID NO:7 or a variant thereof that has a sequence having one amino acid substitution relative to the sequence of SEQ ID NO:7, and an HCDR3 having the sequence of SEQ ID NO:12 or a variant thereof that has a sequence having one amino acid substitution relative to the sequence of SEQ ID NO:12. In some embodiments, an antibody of the disclosure can comprise an HCDR1 having the sequence of SEQ ID NO:3 or a variant thereof that has a sequence having one amino acid substitution relative to the sequence of SEQ ID NO:3, an HCDR2 having the sequence of SEQ ID NO:8 or a variant thereof that has a sequence having one amino acid substitution relative to the sequence of SEQ ID NO:8, and an HCDR3 having the sequence of SEQ ID NO:13 or a variant thereof that has a sequence having one amino acid substitution relative to the sequence of SEQ ID NO:13. In some embodiments, an antibody of the disclosure can comprise an HCDR1 having the sequence of SEQ ID NO:4 or a variant thereof that has a sequence having one amino acid substitution relative to the sequence of SEQ ID NO:4, an HCDR2 having the sequence of SEQ ID NO:9 or a variant thereof that has a sequence having one amino acid substitution relative to the sequence of SEQ ID NO:9, and an HCDR3 having the sequence of SEQ ID NO:14 or a variant thereof that has a sequence having one amino acid substitution relative to the sequence of SEQ ID NO:14. In some embodiments, an antibody of the disclosure can comprise an HCDR1 having the sequence of SEQ ID NO:5 or a variant thereof that has a sequence having one amino acid substitution relative to the sequence of SEQ ID NO:5, an HCDR2 having the sequence of SEQ ID NO:10 or a variant thereof that has a sequence having one amino acid substitution relative to the sequence of SEQ ID NO:10, and an HCDR3 having the sequence of SEQ ID NO:15 or a variant thereof that has a sequence having one amino acid substitution relative to the sequence of SEQ ID NO:15.

An antibody of the disclosure can comprise a heavy chain variable region (V_{H}) having an HCDR1, an HCDR2, and an HCDR3 as described herein. In certain embodiments, an antibody of the disclosure can comprise a heavy chain variable region having at least 90% (e.g., 90%, 91%, 92%, 93%, 94%, 95%, 96%, 97%, 98%, 99%, or 100%) identity to the sequence of any one of SEQ ID NOS: 16-20. In certain embodiments, an antibody of the disclosure can comprise a heavy chain variable region having an HCDR1, an HCDR2, and an HCDR3 of SEQ ID NOS: 1, 6, and 11, respectively, and at least 90% (e.g., 90%, 91%, 92%, 93%, 94%, 95%, 96%, 97%, 98%, 99%, or 100%) identity to the sequence of SEQ ID NO:16. In certain embodiments, an antibody of the disclosure can comprise a heavy chain variable region having an HCDR1, an HCDR2, and an HCDR3 of SEQ ID NOS: 2, 7, and 12, respectively, and at least 90% (e.g., 90%, 91%, 92%, 93%, 94%, 95%, 96%, 97%, 98%, 99%, or 100%) identity to the sequence of SEQ ID NO:17. In certain embodiments, an antibody of the disclosure can comprise a heavy chain variable region having an HCDR1, an HCDR2, and an HCDR3 of SEQ ID NOS: 3, 8, and 13, respectively, and at least 90% (e.g., 90%, 91%, 92%, 93%, 94%, 95%, 96%, 97%, 98%, 99%, or 100%) identity to the sequence of SEQ ID NO:18. In certain embodiments, an antibody of the disclosure can comprise a heavy chain variable region having an HCDR1, an HCDR2, and an HCDR3 of SEQ ID NOS: 4, 9, and 14, respectively, and at least 90% (e.g., 90%, 91%, 92%, 93%, 94%, 95%, 96%, 97%, 98%, 99%, or 100%) identity to the sequence of SEQ ID NO:19. In certain embodiments, an antibody of the disclosure can comprise a heavy chain variable region having an HCDR1, an HCDR2, and an HCDR3 of SEQ ID NOS: 5, 10, and 15, respectively, and at least 90% (e.g., 90%, 91%, 92%, 93%, 94%, 95%, 96%, 97%, 98%, 99%, or 100%) identity to the sequence of SEQ ID NO:20.

### LCDR1-3 and V_{L}

In some embodiments, an antibody of the disclosure can comprise an LCDR1 having the sequence of SEQ ID NO:21 or a variant thereof that has a sequence having one amino acid substitution relative to the sequence of SEQ ID NO:21, an LCDR2 having the sequence of SEQ ID NO:25 or a variant thereof that has a sequence having one amino acid substitutions relative to the sequence of SEQ ID NO:25, and an LCDR3 having the sequence of SEQ ID NO:28 or a variant thereof that has a sequence having one amino acid substitution relative to the sequence of SEQ ID NO:28. In some embodiments, an antibody of the disclosure can comprise an LCDR1 having the sequence of SEQ ID NO:22 or a variant thereof that has a sequence having one amino acid substitution relative to the sequence of SEQ ID NO:22, an LCDR2 having the sequence of SEQ ID NO:25 or a variant thereof that has a sequence having one amino acid substitutions relative to the sequence of SEQ ID NO:25, and an LCDR3 having the sequence of SEQ ID NO:29 or a variant thereof that has a sequence having one amino acid substitution relative to the sequence of SEQ ID NO:29. In some embodiments, an antibody of the disclosure can comprise an LCDR1 having the sequence of SEQ ID NO:21 or a variant thereof that has a sequence having one amino acid substitution relative to the sequence of SEQ ID NO:21, an LCDR2 having the sequence of SEQ ID NO:25 or a variant thereof that has a sequence having one amino acid substitutions relative to the sequence of SEQ ID NO:25, and an LCDR3 having the sequence of SEQ ID NO:30 or a variant thereof that has a sequence having one amino acid substitution relative to the sequence of SEQ ID NO:30. In some embodiments, an antibody of the disclosure can comprise an LCDR1 having the sequence of SEQ ID NO:23 or a variant thereof that has a sequence having one amino acid substitution relative to the sequence of SEQ ID NO:23, an LCDR2 having the sequence of SEQ ID NO:26 or a variant thereof that has a sequence having one amino acid substitutions relative to the sequence of SEQ ID NO:26, and an LCDR3 having the sequence of SEQ ID NO:31 or a variant thereof that has a sequence having one amino acid substitution relative to the sequence of SEQ ID NO:31. In some embodiments, an antibody of the disclosure can comprise an LCDR1 having the sequence of SEQ ID NO:24 or a variant thereof that has a sequence having one amino acid substitution relative to the sequence of SEQ ID NO:24, an LCDR2 having the sequence of SEQ ID NO:27 or a variant thereof that has a sequence having one amino acid substitutions relative to the sequence of SEQ ID NO:27, and an LCDR3 having the sequence of SEQ ID NO:32 or a variant thereof that has a sequence having one amino acid substitution relative to the sequence of SEQ ID NO:32.

An antibody of the disclosure can comprise a light chain variable region (V_{L}) having a LCDR1, a LCDR2, and a LCDR3 as described herein. In certain embodiments, an antibody of the disclosure can comprise a light chain variable region having at least 90% (e.g., 90%, 91%, 92%, 93%, 94%, 95%, 96%, 97%, 98%, 99%, or 100%) identity to the sequence of any one of SEQ ID NOS: 33-37. In certain embodiments, an antibody of the disclosure can comprise a light chain variable region having a LCDR1, a LCDR2, and a LCDR3 of SEQ ID NOS: 21, 25, and 28, respectively, and at least 90% (e.g., 90%, 91%, 92%, 93%, 94%, 95%, 96%, 97%, 98%, 99%, or 100%) identity to the sequence of SEQ ID NO:33. In certain embodiments, an antibody of the disclosure can comprise a light chain variable region having a LCDR1, a LCDR2, and a LCDR3 of SEQ ID NOS: 22, 25, and 29, respectively, and at least 90% (e.g., 90%, 91%, 92%, 93%, 94%, 95%, 96%, 97%, 98%, 99%, or 100%) identity to the sequence of SEQ ID NO:34. In certain embodiments, an antibody of the disclosure can comprise a light chain variable region having a LCDR1, a LCDR2, and a LCDR3 of SEQ ID NOS: 21, 25, and 30, respectively, and at least 90% (e.g., 90%, 91%, 92%, 93%, 94%, 95%, 96%, 97%, 98%, 99%, or 100%) identity to the sequence of SEQ ID NO:35. In certain embodiments, an antibody of the disclosure can comprise a light chain variable region having a LCDR1, a LCDR2, and a LCDR3 of SEQ ID NOS: 23, 26, and 31, respectively, and at least 90% (e.g., 90%, 91%, 92%, 93%, 94%, 95%, 96%, 97%, 98%, 99%, or 100%) identity to the sequence of SEQ ID NO:36. In certain embodiments, an antibody of the disclosure can comprise a light chain variable region having a LCDR1, a LCDR2, and a LCDR3 of SEQ ID NOS: 24, 27, and 32, respectively, and at least 90% (e.g., 90%, 91%, 92%, 93%, 94%, 95%, 96%, 97%, 98%, 99%, or 100%) identity to the sequence of SEQ ID NO:37.

### A100

In particular embodiments, an antibody of the disclosure can comprise: (1) an HCDR1 having the sequence of SEQ ID NO:1 or a sequence having one amino acid substitution relative to the sequence of SEQ ID NO:1; (2) an HCDR2 having the sequence of SEQ ID NO:6 or a sequence having one amino acid substitution relative to the sequence of SEQ ID NO:6; (3) an HCDR3 having the sequence of SEQ ID NO:11 or a sequence having one amino acid substitutions relative to the sequence of SEQ ID NO:11; (4) a LCDR1 having the sequence of SEQ ID NO:21 or a sequence having one amino acid substitution relative to the sequence of SEQ ID NO:21; (5) a LCDR2 having the sequence of SEQ ID NO:25 or a sequence having one amino acid substitution relative to the sequence of SEQ ID NO:25; and (6) a LCDR3 having the sequence of SEQ ID NO:28 or a sequence having one amino acid substitution relative to the sequence of SEQ ID NO:28. In particular embodiments, an antibody of the disclosure can comprise: (1) an HCDR1 having the sequence of SEQ ID NO:1; (2) an HCDR2 having the sequence of SEQ ID NO:6; (3) an HCDR3 having the sequence of SEQ ID NO:11; (4) a LCDR1 having the sequence of SEQ ID NO:21; (5) a LCDR2 having the sequence of SEQ ID NO:25; and (6) a LCDR3 having the sequence of SEQ ID NO:28.

In some embodiments, the antibody can comprise (1) a heavy chain variable region having an HCDR1, an HCDR2, and an HCDR3 of SEQ ID NOS: 1, 6, and 11, respectively, and at least 90% (e.g., 90%, 91%, 92%, 93%, 94%, 95%, 96%, 97%, 98%, 99%, or 100%) identity to the sequence of SEQ ID NO:16, and (2) a light chain variable region having a LCDR1, a LCDR2, and a LCDR3 of SEQ ID NOS: 21, 25, and 28, respectively, and at least 90% (e.g., 90%, 91%, 92%, 93%, 94%, 95%, 96%, 97%, 98%, 99%, or 100%) identity to the sequence of SEQ ID NO:33.

In certain embodiments, the antibody comprises a heavy chain having at least 90% (e.g., 90%, 91%, 92%, 93%, 94%, 95%, 96%, 97%, 98%, 99%, or 100%) identity to the sequence of SEQ ID NO:47: EVQLLESGGGVVQPGRSLRLSCAASGFTFSSYGMHWVRQAPGKGLEWLSGISGSAD TTYYADSVKGRFTISRDNSKNTLYLQMTSLRAEDTAVYYCVRDDIQLRDWGQGTLV TVSSASTKGPSVFPLAPSSKSTSGGTAALGCLVKDYFPEPVTVSWNSGALTSGVHTFP AVLQSSGLYSLSSVVTVPSSSLGTQTYICNVNHKPSNTKVDKRVEPKSCDKTHTCPPC PAPELLGGPSVFLFPPKPKDTLMISRTPEVTCVVVDVSHEDPEVKFNWYVDGVEVHN AKTKPREEQYNSTYRVVSVLTVLHQDWLNGKEYKCKVSNKALPAPIEKTISKAKGQ PREPQVYTLPPSRDELTKNQVSLTCLVKGFYPSDIAVEWESNGQPENNYKTTPPVLDS DGSFFLYSKLTVDKSRWQQGNVFSCSVMHEALHNHYTQKSLSLSPGK, and a light chain having at least 90% (e.g., 90%, 91%, 92%, 93%, 94%, 95%, 96%, 97%, 98%, 99%, or 100%) identity to the sequence of SEQ ID NO:48:

DIQMTQSPSSLSASVGDRVTITCQASQDISNYLNWYQQKPGKAPKLLIYD
ASNLETGVPSRFSGSGSGTDFALTISSLQPEDFATYYCLQDYNYPLTFGG
GTKVDIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In certain embodiments, the antibody comprises (i) a heavy chain comprising a heavy chain variable region having the sequence of SEQ ID NO:16 and a sequence having at least 90% (e.g., 90%, 91%, 92%, 93%, 94%, 95%, 96%, 97%, 98%, 99%, or 100%) identity to the sequence of SEQ ID NO:47; and (ii) a light chain comprising a light chain variable region having the sequence of SEQ ID NO:33 and a sequence having at least 90% (e.g., 90%, 91%, 92%, 93%, 94%, 95%, 96%, 97%, 98%, 99%, or 100%) identity to the sequence of SEQ ID NO:48.

### A101

In particular embodiments, an antibody of the disclosure can comprise: (1) an HCDR1 having the sequence of SEQ ID NO:2 or a sequence having one amino acid substitution relative to the sequence of SEQ ID NO:2; (2) an HCDR2 having the sequence of SEQ ID NO:7 or a sequence having one amino acid substitution relative to the sequence of SEQ ID NO:7; (3) an HCDR3 having the sequence of SEQ ID NO:12 or a sequence having one amino acid substitutions relative to the sequence of SEQ ID NO:12; (4) a LCDR1 having the sequence of SEQ ID NO:22 or a sequence having one amino acid substitution relative to the sequence of SEQ ID NO:22; (5) a LCDR2 having the sequence of SEQ ID NO:25 or a sequence having one amino acid substitution relative to the sequence of SEQ ID NO:25; and (6) a LCDR3 having the sequence of SEQ ID NO:29 or a sequence having one amino acid substitution relative to the sequence of SEQ ID NO:29. In particular embodiments, an antibody of the disclosure can comprise: (1) an HCDR1 having the sequence of SEQ ID NO:2; (2) an HCDR2 having the sequence of SEQ ID NO:7; (3) an HCDR3 having the sequence of SEQ ID NO:12; (4) a LCDR1 having the sequence of SEQ ID NO:22; (5) a LCDR2 having the sequence of SEQ ID NO:25; and (6) a LCDR3 having the sequence of SEQ ID NO:29.

In some embodiments, the antibody can comprise (1) a heavy chain variable region having an HCDR1, an HCDR2, and an HCDR3 of SEQ ID NOS: 2, 7, and 12, respectively, and at least 90% (e.g., 90%, 91%, 92%, 93%, 94%, 95%, 96%, 97%, 98%, 99%, or 100%) identity to the sequence of SEQ ID NO:17, and (2) a light chain variable region having a LCDR1, a LCDR2, and a LCDR3 of SEQ ID NOS: 22, 25, and 29, respectively, and at least 90% (e.g., 90%, 91%, 92%, 93%, 94%, 95%, 96%, 97%, 98%, 99%, or 100%) identity to the sequence of SEQ ID NO:34.

In certain embodiments, the antibody comprises a heavy chain having at least 90% (e.g., 90%, 91%, 92%, 93%, 94%, 95%, 96%, 97%, 98%, 99%, or 100%) identity to the sequence of SEQ ID NO:49: QVQLVQSGAEVKKPGASVKVSCKASGYTFTSYGISWVRQAPGQGLEWMGWISAYN GNTNYAQKLQGRVTMTTDTSTSTAYMELRSLRSDDTAVYYCARDYSGSWYPSNGP ALDYWGQGTMVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDKRVE PKSCDKTHTCPPCPAPELLGGPSVFLFPPKPKDTLMISRTPEVTCVVVDVSHEDPEVK FNWYVDGVEVHNAKTKPREEQYNSTYRVVSVLTVLHQDWLNGKEYKCKVSNKAL PAPIEKTISKAKGQPREPQVYTLPPSRDELTKNQVSLTCLVKGFYPSDIAVEWESNGQ PENNYKTTPPVLDSDGSFFLYSKLTVDKSRWQQGNVFSCSVMHEALHNHYTQKSLS LSPGK, and a light chain having at least 90% (e.g., 90%, 91%, 92%, 93%, 94%, 95%, 96%, 97%, 98%, 99%, or 100%) identity to the sequence of SEQ ID NO:50:

DIQMTQSPSSLSASVGDRVTITCRASQYISSYLNWYQQKPGKAPKLLIYD
ASNLETGVPSRFSGSGSGTDFTFTISSLQPEDIATYYCLQDYSYPLTFGG
GIKVDIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In certain embodiments, the antibody comprises (i) a heavy chain comprising a heavy chain variable region having the sequence of SEQ ID NO:17 and a sequence having at least 90% (e.g., 90%, 91%, 92%, 93%, 94%, 95%, 96%, 97%, 98%, 99%, or 100%) identity to the sequence of SEQ ID NO:49; and (ii) a light chain comprising a light chain variable region having the sequence of SEQ ID NO:34 and a sequence having at least 90% (e.g., 90%, 91%, 92%, 93%, 94%, 95%, 96%, 97%, 98%, 99%, or 100%) identity to the sequence of SEQ ID NO:50.

### A102

In particular embodiments, an antibody of the disclosure can comprise: (1) an HCDR1 having the sequence of SEQ ID NO:3 or a sequence having one amino acid substitution relative to the sequence of SEQ ID NO:3; (2) an HCDR2 having the sequence of SEQ ID NO:8 or a sequence having one amino acid substitution relative to the sequence of SEQ ID NO:8; (3) an HCDR3 having the sequence of SEQ ID NO:13 or a sequence having one amino acid substitutions relative to the sequence of SEQ ID NO:13; (4) a LCDR1 having the sequence of SEQ ID NO:21 or a sequence having one amino acid substitution relative to the sequence of SEQ ID NO:21; (5) a LCDR2 having the sequence of SEQ ID NO:25 or a sequence having one amino acid substitution relative to the sequence of SEQ ID NO:25; and (6) a LCDR3 having the sequence of SEQ ID NO:30 or a sequence having one amino acid substitution relative to the sequence of SEQ ID NO:30. In particular embodiments, an antibody of the disclosure can comprise: (1) an HCDR1 having the sequence of SEQ ID NO:3; (2) an HCDR2 having the sequence of SEQ ID NO:8; (3) an HCDR3 having the sequence of SEQ ID NO:13; (4) a LCDR1 having the sequence of SEQ ID NO:21; (5) a LCDR2 having the sequence of SEQ ID NO:25; and (6) a LCDR3 having the sequence of SEQ ID NO:30.

In some embodiments, the antibody can comprise (1) a heavy chain variable region having an HCDR1, an HCDR2, and an HCDR3 of SEQ ID NOS: 3, 8, and 13, respectively, and at least 90% (e.g., 90%, 91%, 92%, 93%, 94%, 95%, 96%, 97%, 98%, 99%, or 100%) identity to the sequence of SEQ ID NO:18, and (2) a light chain variable region having a LCDR1, a LCDR2, and a LCDR3 of SEQ ID NOS: 21, 25, and 30, respectively, and at least 90% (e.g., 90%, 91%, 92%, 93%, 94%, 95%, 96%, 97%, 98%, 99%, or 100%) identity to the sequence of SEQ ID NO:35.

In certain embodiments, the antibody comprises a heavy chain having at least 90% (e.g., 90%, 91%, 92%, 93%, 94%, 95%, 96%, 97%, 98%, 99%, or 100%) identity to the sequence of SEQ ID NO:51: EVQLVQSGAEVKKPGASVKVSCKTSGFTFTNYGISWVRQAPGQGLEWMGWISANN GNSNYAQDHQGRVTMTTDTSTSTAYMELRSLRSDDTAVYYCAREFPGWYFDYWG QGTLVTVSSASTKGPSVFPLAPSSKSTSGGTAALGCLVKDYFPEPVTVSWNSGALTS GVHTFPAVLQSSGLYSLSSVVTVPSSSLGTQTYICNVNHKPSNTKVDKRVEPKSCDK THTCPPCPAPELLGGPSVFLFPPKPKDTLMISRTPEVTCVVVDVSHEDPEVKFNWYVD GVEVHNAKTKPREEQYNSTYRVVSVLTVLHQDWLNGKEYKCKVSNKALPAPIEKTI SKAKGQPREPQVYTLPPSRDELTKNQVSLTCLVKGFYPSDIAVEWESNGQPENNYKT TPPVLDSDGSFFLYSKLTVDKSRWQQGNVFSCSVMHEALHNHYTQKSLSLSPGK, and a light chain having at least 90% (e.g., 90%, 91%, 92%, 93%, 94%, 95%, 96%, 97%, 98%, 99%, or 100%) identity to the sequence of SEQ ID NO:52:

DIQMTQSPSSLSASVGDRVTITCQASQDISNYLNWYQQKPGKAPKLLIYD
ASNLETGVPSRFSGSGSGTDFTFTISSLQPDDFATYYCQQSYRTPPTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In certain embodiments, the antibody comprises (i) a heavy chain comprising a heavy chain variable region having the sequence of SEQ ID NO:18 and a sequence having at least 90% (e.g., 90%, 91%, 92%, 93%, 94%, 95%, 96%, 97%, 98%, 99%, or 100%) identity to the sequence of SEQ ID NO:51; and (ii) a light chain comprising a light chain variable region having the sequence of SEQ ID NO:35 and a sequence having at least 90% (e.g., 90%, 91%, 92%, 93%, 94%, 95%, 96%, 97%, 98%, 99%, or 100%) identity to the sequence of SEQ ID NO:52.

### A103

In particular embodiments, an antibody of the disclosure can comprise: (1) an HCDR1 having the sequence of SEQ ID NO:4 or a sequence having one amino acid substitution relative to the sequence of SEQ ID NO:4; (2) an HCDR2 having the sequence of SEQ ID NO:9 or a sequence having one amino acid substitution relative to the sequence of SEQ ID NO:9; (3) an HCDR3 having the sequence of SEQ ID NO:14 or a sequence having one amino acid substitutions relative to the sequence of SEQ ID NO:14; (4) a LCDR1 having the sequence of SEQ ID NO:23 or a sequence having one amino acid substitution relative to the sequence of SEQ ID NO:23; (5) a LCDR2 having the sequence of SEQ ID NO:26 or a sequence having one amino acid substitution relative to the sequence of SEQ ID NO:26; and (6) a LCDR3 having the sequence of SEQ ID NO:31 or a sequence having one amino acid substitution relative to the sequence of SEQ ID NO:31. In particular embodiments, an antibody of the disclosure can comprise: (1) an HCDR1 having the sequence of SEQ ID NO:4; (2) an HCDR2 having the sequence of SEQ ID NO:9; (3) an HCDR3 having the sequence of SEQ ID NO:14; (4) a LCDR1 having the sequence of SEQ ID NO:23; (5) a LCDR2 having the sequence of SEQ ID NO:26; and (6) a LCDR3 having the sequence of SEQ ID NO:31.

In some embodiments, the antibody can comprise (1) a heavy chain variable region having an HCDR1, an HCDR2, and an HCDR3 of SEQ ID NOS: 4, 9, and 14, respectively, and at least 90% (e.g., 90%, 91%, 92%, 93%, 94%, 95%, 96%, 97%, 98%, 99%, or 100%) identity to the sequence of SEQ ID NO:19, and (2) a light chain variable region having a LCDR1, a LCDR2, and a LCDR3 of SEQ ID NOS: 23, 26, and 31, respectively, and at least 90% (e.g., 90%, 91%, 92%, 93%, 94%, 95%, 96%, 97%, 98%, 99%, or 100%) identity to the sequence of SEQ ID NO:36.

In certain embodiments, the antibody comprises a heavy chain having at least 90% (e.g., 90%, 91%, 92%, 93%, 94%, 95%, 96%, 97%, 98%, 99%, or 100%) identity to the sequence of SEQ ID NO:53: EVOLVESGGGLVQPGGSLRLSCAASGFTFSSYSMNWVRQAPGKGLEWVSSISSSSSYI YYADSVKGRFTISRDNSKNTVYLQMNSLRAEDTAVYYCAREYYDFWSGYPSGYAF DIWGQGTLVTVSSASTKGPSVFPLAPSSKSTSGGTAALGCLVKDYFPEPVTVSWNSG ALTSGVHTFPAVLQSSGLYSLSSVVTVPSSSLGTQTYICNVNHKPSNTKVDKRVEPKS CDKTHTCPPCPAPELLGGPSVFLFPPKPKDTLMISRTPEVTCVVVDVSHEDPEVKFNW YVDGVEVHNAKTKPREEQYNSTYRVVSVLTVLHQDWLNGKEYKCKVSNKALPAPI EKTISKAKGQPREPQVYTLPPSRDELTKNQVSLTCLVKGFYPSDIAVEWESNGQPEN NYKTTPPVLDSDGSFFLYSKLTVDKSRWQQGNVFSCSVMHEALHNHYTQKSLSLSP GK, and a light chain having at least 90% (e.g., 90%, 91%, 92%, 93%, 94%, 95%, 96%, 97%, 98%, 99%, or 100%) identity to the sequence of SEQ ID NO:54:

QSALTQDPAVSVALGQTVRITCQGDSLRSYYASWYQQKPGQAPVLVIYGK
NNRPSGIPDRFSGSSSGNTASLTITGAQAEDEADYYCNSRDSSGNHWVFG
GGTKLTVLRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

In certain embodiments, the antibody comprises (i) a heavy chain comprising a heavy chain variable region having the sequence of SEQ ID NO:19 and a sequence having at least 90% (e.g., 90%, 91%, 92%, 93%, 94%, 95%, 96%, 97%, 98%, 99%, or 100%) identity to the sequence of SEQ ID NO:53; and (ii) a light chain comprising a light chain variable region having the sequence of SEQ ID NO:36 and a sequence having at least 90% (e.g., 90%, 91%, 92%, 93%, 94%, 95%, 96%, 97%, 98%, 99%, or 100%) identity to the sequence of SEQ ID NO:54.

### A104

In particular embodiments, an antibody of the disclosure can comprise: (1) an HCDR1 having the sequence of SEQ ID NO:5 or a sequence having one amino acid substitution relative to the sequence of SEQ ID NO:5; (2) an HCDR2 having the sequence of SEQ ID NO:10 or a sequence having one amino acid substitution relative to the sequence of SEQ ID NO:10; (3) an HCDR3 having the sequence of SEQ ID NO:15 or a sequence having one amino acid substitutions relative to the sequence of SEQ ID NO:15; (4) a LCDR1 having the sequence of SEQ ID NO:24 or a sequence having one amino acid substitution relative to the sequence of SEQ ID NO:24; (5) a LCDR2 having the sequence of SEQ ID NO:27 or a sequence having one amino acid substitution relative to the sequence of SEQ ID NO:27; and (6) a LCDR3 having the sequence of SEQ ID NO:32 or a sequence having one amino acid substitution relative to the sequence of SEQ ID NO:32. In particular embodiments, an antibody of the disclosure can comprise: (1) an HCDR1 having the sequence of SEQ ID NO:5; (2) an HCDR2 having the sequence of SEQ ID NO:10; (3) an HCDR3 having the sequence of SEQ ID NO:15; (4) a LCDR1 having the sequence of SEQ ID NO:24; (5) a LCDR2 having the sequence of SEQ ID NO:27; and (6) a LCDR3 having the sequence of SEQ ID NO:32.

In some embodiments, the antibody can comprise (1) a heavy chain variable region having an HCDR1, an HCDR2, and an HCDR3 of SEQ ID NOS: 5, 10, and 15, respectively, and at least 90% (e.g., 90%, 91%, 92%, 93%, 94%, 95%, 96%, 97%, 98%, 99%, or 100%) identity to the sequence of SEQ ID NO:20, and (2) a light chain variable region having a LCDR1, a LCDR2, and a LCDR3 of SEQ ID NOS: 24, 27, and 32, respectively, and at least 90% (e.g., 90%, 91%, 92%, 93%, 94%, 95%, 96%, 97%, 98%, 99%, or 100%) identity to the sequence of SEQ ID NO:37.

In certain embodiments, the antibody comprises a heavy chain having at least 90% (e.g., 90%, 91%, 92%, 93%, 94%, 95%, 96%, 97%, 98%, 99%, or 100%) identity to the sequence of SEQ ID NO:55: QVQLVQSGAEVKKPGSSVKVSCKASGGTFSSYAINWVRQAPGQGLEWMGGIIPIFGT ANYAQKFQGRVTITADKSTSTAYMELSSLRSEDTAVYYCARGVPSGSGYYLGLDYW GQGTMVTVSSASTKGPSVFPLAPSSKSTSGGTAALGCLVKDYFPEPVTVSWNSGALT SGVHTFPAVLQSSGLYSLSSVVTVPSSSLGTQTYICNVNHKPSNTKVDKRVEPKSCDK THTCPPCPAPELLGGPSVFLFPPKPKDTLMISRTPEVTCVVVDVSHEDPEVKFNWYVD GVEVHNAKTKPREEQYNSTYRVVSVLTVLHQDWLNGKEYKCKVSNKALPAPIEKTI SKAKGQPREPQVYTLPPSRDELTKNQVSLTCLVKGFYPSDIAVEWESNGQPENNYKT TPPVLDSDGSFFLYSKLTVDKSRWQQGNVFSCSVMHEALHNHYTQKSLSLSPGK, and a light chain having at least 90% (e.g., 90%, 91%, 92%, 93%, 94%, 95%, 96%, 97%, 98%, 99%, or 100%) identity to the sequence of SEQ ID NO:56:

QSELTQPPSASGAPGQRVTISCSGSSSNIGSNYVYWYQQLPGTAPKLLIY
RNNQRPSGVPDRFSGSKSGTSASLAISGLRSEDEADYYCAAWDDSLSGPV
FSGGTKLTVLRTVAAPSVFIFPPSDEQLKSGTASVVCLLNNFYPREAKVQ
WKVDNALQSGNSQESVTEQDSKDSTYSLSSTLTLSKADYEKHKVYACEVT
HQGLSSPVTKSFNRGEC.

In certain embodiments, the antibody comprises (i) a heavy chain comprising a heavy chain variable region having the sequence of SEQ ID NO:20 and a sequence having at least 90% (e.g., 90%, 91%, 92%, 93%, 94%, 95%, 96%, 97%, 98%, 99%, or 100%) identity to the sequence of SEQ ID NO:55; and (ii) a light chain comprising a light chain variable region having the sequence of SEQ ID NO:37 and a sequence having at least 90% (e.g., 90%, 91%, 92%, 93%, 94%, 95%, 96%, 97%, 98%, 99%, or 100%) identity to the sequence of SEQ ID NO:56.

### IV. Fc Polypeptide

An anti-α3β1 antibody provided herein can comprise a fragment crystallizable region (Fc region), also referred to as an Fc polypeptide herein. An Fc polypeptide is part of each of the two heavy chains in the antibody and can interact with certain cell surface receptors and certain components of the complement system. An Fc polypeptide typically includes the CH2 domain and the CH3 domain, which are immunoglobulin constant region domain polypeptides. In some embodiments, the Fc polypeptide in an antibody described herein can be a wild-type Fc polypeptide, e.g., a human IgG1 Fc polypeptide. In certain embodiments, an antibody described herein can comprise a wild-type Fc polypeptide having the sequence of SEQ ID NO:43: APELLGGPSVFLFPPKPKDTLMISRTPEVTCVVVDVSHEDPEVKFNWYVDGVEVHNA KTKPREEQYNSTYRVVSVLTVLHQDWLNGKEYKCKVSNKALPAPIEKTISKAKGQP REPQVYTLPPSRDELTKNQVSLTCLVKGFYPSDIAVEWESNGQPENNYKTTPPVLDS DGSFFLYSKLTVDKSRWQQGNVFSCSVMHEALHNHYTQKSLSLSPGK. In other embodiments, an antibody described herein can comprise a variant of the wild-type Fc polypeptide that has at least 90% (e.g., 90%, 91%, 92%, 93%, 94%, 95%, 96%, 97%, 98%, 99%, or 99.5%) identity to the sequence of a wild-type Fc polypeptide (e.g., SEQ ID NO:43) and at least one amino acid substitution relative to the sequence of a wild-type Fc polypeptide (e.g., SEQ ID NO:43).

In some embodiments, an Fc polypeptide includes one or more modifications (e.g., one or more amino acid substitutions, insertions, or deletions relative to a comparable wild-type Fc region). Antibodies comprising modified Fc polypeptides typically have altered phenotypes relative to antibodies comprising wild-type Fc polypeptides. For example, antibodies comprising modified Fc polypeptides can have altered serum half-life, altered stability, altered susceptibility to cellular enzymes, and/or altered effector function (e.g., as assayed in an NK-dependent or macrophage-dependent assay).

In some embodiments, an Fc polypeptide in an antibody described herein can include amino acid substitutions that modulate effector function. In certain embodiments, an Fc polypeptide in an antibody described herein can include amino acid substitutions that reduce or eliminate effector function. Illustrative Fc polypeptide amino acid substitutions that reduce effector function include, but are not limited to, substitutions in a CH2 domain, e.g., at positions 4 and 5 (position numbering relative to the sequence of SEQ ID NO:43) (see, e.g., Lund et al., J Immunol. 147(8):2657-62, 1991). For example, in some embodiments, one or both Fc polypeptides in an antibody described herein can comprise L4A and L5A substitutions.

Additional Fc polypeptide amino acid substitutions that modulate an effector function include, e.g., substitution at position 99 (position numbering relative to the sequence of SEQ ID NO:43). For example, in some embodiments, one or both Fc polypeptides in an antibody described herein can comprise P99G substitution. In certain embodiments, one or both Fc polypeptides in an antibody described herein can have L4A, L5A, and P99G substitutions.

In some embodiments, an Fc polypeptide includes one or more modifications that alter (relative to a wild-type Fc polypeptide) the Ratio of Affinities of the modified Fc polypeptide to an activating FcγR (such as FcγRIIA or FcγRIIIA) relative to an inhibiting FcγR (such as FcγRIIB):

$$Ratio of Affinities = \frac{Wild - Type to Variant Change in Affinity to Fc γ {R}_{Activating}}{Wild - Type to Variant Change in Affinity to Fc γ {R}_{Inhibiting}} .$$

Where a modified Fc polypeptide has a ratio of affinities greater than 1, an anti-α3β1 antibody herein may have particular use in providing a therapeutic or prophylactic treatment of a disease, disorder, or infection, or the amelioration of a symptom thereof, where an enhanced efficacy of effector cell function (e.g., ADCC) mediated by FcγR is desired, e.g., cancer or infectious disease. Where a modified Fc region has a ratio of affinities less than 1, an anti-α3β1 antibody herein may have particular use in providing a therapeutic or prophylactic treatment of a disease or disorder, or the amelioration of a symptom thereof, where a decreased efficacy of effector cell function mediated by FcγR is desired, e.g., autoimmune or inflammatory disorders. Table 2 lists examples of single, double, triple, quadruple, and quintuple amino acid substitutions in an Fc polypeptide that provide a Ratio of Affinities greater than 1 or less than 1 (see e.g., PCT Publication Nos. WO 04/063351; WO 06/088494; WO 07/024249; WO 06/113665; WO 07/021841; WO 07/106707; WO 2008/140603). Amino acid positions are numbered according to EU numbering scheme.

TABLE 2

| Ratio | Single | Double | Triple | Quadruple | Quintuple |
|---|---|---|---|---|---|
| >1 | F243L | F243L & R292P | F243L, P247L & | L234F, F243L, | L235V, F243L, R292P, |
| | D270E | F243L & Y300L | N421K | R292P & Y300L | Y300L & P396L |
| | R292G | F243L & P396L | F243L, R292P & | L235I, F243L, | L235P, F243L, R292P, |
| | R292P | D270E & P396L | Y300L | R292P & Y300L | Y300L & P396L |
| | | R292P & Y300L | F243L, R292P & | L235Q, F243L, | F243L, R292P, V305I, |
| | | R292P & V305I | V305I | R292P & Y300L | Y300L & P396L |
| | | R292P & P396L | F243L, R292P & | F243L, P247L, | |
| | | Y300L & P396L | P396L | D270E & N421K | |
| | | P396L & Q419H | F243L, Y300L & | F243L, R255L, | |
| | | | P396L | D270E & P396L | |
| | | | P247L, D270E & | F243L, D270E, | |
| | | | N421K | G316D & R416G | |
| | | | R255L, D270E & | F243L, D270E, | |
| | | | P396L | K392T & P396L | |
| | | | D270E, G316D & | F243L, D270E, | |
| | | | R416G | P396L & Q419H | |
| | | | D270E, K392T & | F243L, R292P, | |
| | | | P396L | Y300L, & P396L | |
| | | | D270E, P396L & | F243L, R292P, | |
| | | | Q419H | V305I & P396L | |
| | | | V284M, R292L & | P247L, D270E, | |
| | | | K370N | Y300L & N421K | |
| | | | R292P, Y300L & | R255L, D270E, | |
| | | | P396L | R292G & P396L | |
| | | | | R255L, D270E, | |
| | | | | Y300L & P396L | |
| | | | | D270E, G316D, | |
| | | | | P396L & R416G | |
| <1 | Y300L | F243L & P396L | F243L, R292P & | | |
| | P396L | P247L & N421K | V305I | | |
| | | R255L & P396L | | | |
| | | R292P & V305I | | | |
| | | K392T & P396L | | | |
| | | P396L & Q419H | | | |

### V. Antibodies that Competitively Bind with an Anti-α3β1 Antibody

Also provided herein are anti-α3β1 antibodies that competitively bind, or are capable of competitively binding (e.g., competitor antibodies), with one or more anti-α3β1 antibodies described herein. In certain instances, an antibody (i.e., competitor antibody) may be considered to compete for binding to α3β1 when the competitor binds to the same general region of α3β1 as an anti-α3β1 antibody described herein. In certain instances, an antibody (i.e., competitor antibody) may be considered to compete for binding to α3β1 when the competitor binds to the exact same region of α3β1 as an anti-α3β1 antibody described herein (e.g., exact same peptide (linear epitope) or exact same surface amino acids (conformational epitope)). In certain instances, an antibody (i.e., competitor antibody) may be considered capable of competing for binding to α3β1 when the competitor binds to the same general region of α3β1 as an anti-α3β1 antibody described herein (i.e., a sequence within a thigh-genu region of integrin α3β1) under suitable assay conditions. In certain instances, an antibody (i.e., competitor agent) may be considered capable of competing for binding to α3β1 when the competitor binds to the exact same region of α3β1 as an anti-α3β1 antibody described herein (e.g., exact same peptide (linear epitope) or exact same surface amino acids (conformational epitope)) under suitable assay conditions.

In certain instances, an antibody (i.e., competitor antibody) may be considered to compete for binding to α3β1 when the competitor blocks the binding of one or more anti-α3β1 antibodies described herein to α3β1, for example, under suitable assay conditions. Whether a competitor blocks the binding of one or more anti-α3β1 antibodies described herein to α3β1 may be determined using a suitable competition assay or blocking assay, such as, for example, a blocking assay as described in herein. A competitor antibody may block binding of one or more anti-α3β1 antibodies described herein to α3β1 in a competition or blocking assay by 50% or more (e.g., 55%, 60%, 65%, 70%, 75%, 80%, 85%, 90%, 95% or more, or 100%), and conversely, one or more anti-α3β1 antibodies described herein may block binding of the competitor antibody to α3β1 in a competition or blocking assay by about 50% or more (e.g., e.g., 55%, 60%, 65%, 70%, 75%, 80%, 85%, 90%, 95% or more, or 100%).

In certain instances, an antibody (i.e., competitor antibody) may be considered to compete for binding to α31 when the competitor binds to α3β1 with a similar affinity as one or more anti-α3β1 antibodies described herein, for example, under suitable assay conditions. In some embodiments, an antibody (i.e., competitor antibody) is considered to compete for binding to α3β1 when the competitor binds to α31 with an affinity that is at least about 50% (e.g., 55%, 60%, 65%, 70%, 75%, 80%, 85%, 90%, 95%, or 100%) of the affinity of one or more anti-α3β1 antibodies described herein.

Also provided herein are anti-α3β1 antibodies that bind to, or are capable of binding to, the same epitope as one or more anti-α3β1 antibodies described herein. In particular, provided herein are anti-α3β1 antibodies that compete with one or more anti-α3β1 antibodies described herein for binding to the same epitope (e.g., same peptide (linear epitope) or same surface amino acids (conformational epitope)) on α3β1. Such antibodies that bind the same epitope may be referred to as epitope competitors.

### VI. Polyclonal and Monoclonal Antibodies

Polyclonal antibodies may be raised in animals (vertebrate or invertebrates, including mammals, birds and fish, including cartilaginous fish) by multiple subcutaneous (sc) or intraperitoneal (ip) injections of a relevant antigen and an adjuvant. It may be useful to conjugate the relevant antigen to a protein or other carrier that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl2, or R1N═C═NR, where R and R1 are different alkyl groups. Non-protein carriers (e.g., colloidal gold) also may be used for antibody production.

Animals can be immunized against the antigen, immunogenic conjugates, or derivatives by combining, e.g., 100 μg or 5 μg of the protein or conjugate (for rabbits or mice, respectively) with three volumes of Freund's complete adjuvant and injecting the solution intradermally at multiple sites. One month later the animals are boosted with one-fifth to one-tenth of the original amount of peptide or conjugate in Freund's complete adjuvant by subcutaneous injection at multiple sites. Seven to 14 days later the animals are bled and the serum is assayed for antibody titer. Animals are boosted until the titer plateaus. Often, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal antibodies may be made using a hybridoma, e.g., the hybridoma method first described by Kohler et al., Nature, 256:495 (1975), or may be made by other methods such as recombinant DNA methods (see, e.g., U.S. Pat. No. 4,816,567). In the hybridoma method, a mouse or other appropriate host animal, such as a hamster or macaque monkey, is immunized to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (see, e.g., Goding, Monoclonal Antibodies: Principles and Practice, pp. 59-103 (Academic Press, 1986)).

The hybridoma cells thus prepared are seeded and grown in a suitable culture medium that may contain one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells. Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as SP-2 or X₆₃-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. The binding specificity of monoclonal antibodies produced by hybridoma cells may be determined by immunoprecipitation, by an in vitro binding assay, such as radioimmunoassay (RIA) or enzyme-linked immunoabsorbent assay (ELISA), or by flow cytometric analysis of cells expressing the membrane antigen. The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (see, e.g., Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies can be readily isolated and sequenced using conventional procedures (e.g., by using oligonucleotide probes that are capable of binding specifically to genes encoding the heavy and light chains of the monoclonal antibodies). Alternatively, cDNA may be prepared from mRNA and the cDNA then subjected to DNA sequencing. The hybridoma cells serve as a preferred source of such genomic DNA or RNA for cDNA preparation. Once isolated, the DNA may be placed into expression vectors, which are well known in the art, and which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells.

### VII. Humanization and Amino Acid Variants

General methods for humanization of antibodies are described, for example, in U.S. Pat. Nos. 5,861,155, 6,479,284, 6,407,213, 6,639,055, 6,500,931, 5,530,101, 5,585,089, 5,693,761, 5,693,762, 6,180,370, 5,714,350, 6,350,861, 5,777,085, 5,834,597, 5,882,644, 5,932,448, 6,013,256, 6,129,914, 6,210,671, 6,329,511, 5,225,539, 6,548,640, and 5,624,821. In certain embodiments, it may be desirable to generate amino acid sequence variants of these humanized antibodies, particularly where these improve the binding affinity or other biological properties (e.g., half-life) of the antibody.

In some embodiments, the antibody is a humanized antibody, i.e., an antibody that retains the reactivity of a non-human antibody while being less immunogenic in humans. This can be achieved, for instance, by retaining the non-human CDR regions and replacing the remaining parts of the antibody with their human counterparts. See, e.g., Morrison et al., PNAS USA, 81:6851-6855 (1984); Morrison and Oi, Adv. Immunol., 44:65-92 (1988); Verhoeyen et al., Science, 239:1534-1536 (1988); Padlan, Molec. Immun., 28:489-498 (1991); Padlan, Molec. Immun., 31 (3): 169-217 (1994). Techniques for humanizing antibodies are well known in the art and are described in e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534. Humanized antibodies are further described in, e.g., Winter and Milstein (1991) Nature 349:293. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well-known procedures from a variety of human cells. The CDRs for producing the immunoglobulins of the present disclosure can be similarly derived from monoclonal antibodies capable of specifically binding to α3β1.

Amino acid sequence variants of the anti-α3β1 antibody can be prepared by introducing appropriate nucleotide changes into the anti-α3β1 antibody DNA, or by peptide synthesis. Such variants include, for example, deletions from, and/or insertions into and/or substitutions of, residues within the amino acid sequences of the anti-α3β1 antibodies for the examples herein. Any combination of deletion, insertion, and substitution is made to arrive at the final construct, provided that the final construct possesses the desired characteristics. The amino acid changes also may alter post-translational processes of the humanized or variant anti-α3β1 antibody, such as changing the number or position of glycosylation sites.

One method for identification of certain residues or regions of the anti-α3β1 antibody that are preferred locations for mutagenesis is called “alanine scanning mutagenesis,” as described by, e.g., Cunningham and Wells, Science, 244:1081-1085 (1989). Here, a residue or group of target residues are identified (e.g., charged residues such as Arg, Asp, His, Lys, and Glu) and replaced by a neutral or negatively charged amino acid (most preferably Ala or poly-Ala) to affect the interaction of the amino acids with α3β1 antigen (e.g., a sequence within a thigh-genu region of integrin α3β1). Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis is conducted at the target codon or region and the expressed anti-α3β1 antibody variants are screened for the desired activity. Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an N-terminal methionyl residue or the antibody fused to an epitope tag. Other insertional variants include the fusion of an enzyme or a polypeptide that increases the serum half-life of the antibody to the N- or C-terminus of the antibody.

Another type of variant is an amino acid substitution variant. These variants have at least one amino acid residue removed from the antibody molecule and a different residue inserted in its place. The sites of greatest interest for substitutional mutagenesis include the hypervariable regions, but FR alterations are also contemplated. Conservative substitutions are preferred, but more substantial changes may be introduced and the products may be screened. Examples of substitutions are listed below:
Ala (A): Val; Leu; Ile; Val
Arg (R): Lys; Gln; Asn; Lys
Asn (N): Gln; His; Asp, Lys; Gln; Arg
Asp (D): Glu; Asn
Cys (C): Ser; Ala
Gln (Q): Asn; Glu
Glu (E): Asp; Gln
Gly (G): Ala
His (H): Asn; Gln; Lys; Arg
Ile (I): Leu; Val; Met; Ala; Leu; Phe; Norleucine
Leu (L): Norleucine; Ile; Val; Ile; Met; Ala; Phe
Lys (K): Arg; Gln; Asn
Met (M): Leu; Phe; Ile
Phe (F): Leu; Val; Ile; Ala; Tyr
Pro (P): Ala
Ser(S): Thr
Thr (T): Ser
Trp (W): Tyr; Phe
Tyr (Y): Trp; Phe; Thr; Ser
Val (V): Ile; Leu; Met; Phe; Ala; Norleucine

Substantial modifications in the biological properties of an antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: Norleucine, Met, Ala, Val, Leu, Ile;
(2) neutral hydrophilic: Cys, Ser, Thr;
(3) acidic: Asp, Glu;
(4) basic: Asn, Gln, His, Lys, Arg;
(5) residues that influence chain orientation: Gly, Pro; and
(6) aromatic: Trp, Tyr, Phe

Non-conservative substitutions will entail exchanging a member of one of the above classes for another class.

Any cysteine residue not involved in maintaining the proper conformation of the antibody also may be substituted, to improve the oxidative stability of the molecule and prevent aberrant crosslinking. Conversely, cysteine bond(s) may be added to the antibody to improve its stability (particularly where the antibody is an antibody fragment such as an Fv fragment).

One type of substitutional variant involves substituting one or more hypervariable region residues of a parent antibody. Generally, the resulting variant(s) selected for further development will have improved biological properties relative to the parent antibody from which they are generated. A convenient way for generating such substitutional variants is affinity maturation using phage display. Briefly, several hypervariable region sites (e.g., 6-7 sites) are mutated to generate all possible amino acid substitutions at each site. The antibody variants thus generated can be displayed in the monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine-scanning mutagenesis can be performed to identify hypervariable region residues contributing significantly to antigen binding. Alternatively, or in addition, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one of more carbohydrate moieties found in the antibody, and/or adding one or more glycosylation sites that are not present in the antibody. Glycosylation of antibodies is typically either N-linked and/or O-linked. N-linked refers to the attachment of the carbohydrate moiety to the side chain of an asparagine residue. The tripeptide sequences asparagine-X-serine and asparagine-X-threonine, where X is any amino acid except proline, are the most common recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. Addition of glycosylation sites to the antibody can be accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

### VIII. Other Modifications

Other modifications of an anti-α3β1 antibody are contemplated. For example, technology herein also pertains to immunoconjugates comprising an anti-α3β1 antibody described herein conjugated to a cytotoxic agent such as a toxin (e.g., an enzymatically active toxin of bacterial, fungal, plant or animal origin, or fragments thereof), or a radioactive isotope (for example, a radioconjugate), or a cytotoxic drug. Such conjugates are sometimes referred to as “antibody-drug conjugates” or “ADC.” Conjugates can be made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis-(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene).

Anti-α3β1 antibodies (e.g., anti-α3β1 antibodies) disclosed herein may be formulated as immunoliposomes. Liposomes containing an antibody are prepared by methods know in the art, such as described in Epstein et al., Proc. Natl. Acad. Sci. USA 82:3688 (1985); Hwang et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. For example, liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of an antibody provided herein can be conjugated to the liposomes as described in Martin et al., J. Biol. Chem. 257:286-288 (1982) via a disulfide interchange reaction. Another active ingredient is optionally contained within the liposome.

Enzymes or other polypeptides can be covalently bound to an anti-α3β1 antibody by techniques well known in the art such as the use of the heterobifunctional cross-linking reagents discussed above. In some embodiments, fusion proteins comprising at least the antigen binding region of an antibody provided herein linked to at least a functionally active portion of an enzyme can be constructed using recombinant DNA techniques well known in the art (see, e.g., Neuberger et al., Nature 312:604-608 (1984)).

In certain embodiments, it may be desirable to use an antibody fragment, rather than an intact antibody, to increase penetration of target tissues and cells, for example. In such instances, it may be desirable to modify the antibody fragment in order to increase its serum half-life. This may be achieved, for example, by incorporation of a salvage receptor binding epitope into the antibody fragment (e.g., by mutation of the appropriate region in the antibody fragment or by incorporating the epitope into a peptide tag that is then fused to the antibody fragment at either end or in the middle, e.g., by DNA or peptide synthesis; see, e.g., WO96/32478 published Oct. 17, 1996).

In some embodiments, a modification can optionally be introduced into the antibodies (e.g., within the polypeptide chain or at either the N- or C-terminal), e.g., to extend in vivo half-life, such as PEGylation or incorporation of long-chain polyethylene glycol polymers (PEG). Introduction of PEG or long chain polymers of PEG increases the effective molecular weight of the polypeptides, for example, to prevent rapid filtration into the urine. In some embodiments, a lysine residue in the sequence is conjugated to PEG directly or through a linker. Such linker can be, for example, a Glu residue or an acyl residue containing a thiol functional group for linkage to the appropriately modified PEG chain. An alternative method for introducing a PEG chain is to first introduce a Cys residue at the C-terminus or at solvent exposed residues such as replacements for Arg or Lys residues. This Cys residue is then site-specifically attached to a PEG chain containing, for example, a maleimide function. Methods for incorporating PEG or long chain polymers of PEG are known in the art (described, for example, in Veronese, F. M., et al., Drug Disc. Today 10:1451-8 (2005); Greenwald, R. B., et al., Adv. Drug Deliv. Rev. 55:217-50 (2003); Roberts, M. J., et al., Adv. Drug Deliv. Rev., 54:459-76 (2002)), the contents of which are incorporated herein by reference.

Covalent modifications of an anti-α3β1 antibody are also included within the scope of this technology. For example, modifications may be made by chemical synthesis or by enzymatic or chemical cleavage of an anti-α3β1 antibody. Other types of covalent modifications of an antibody are introduced into the molecule by reacting targeted amino acid residues of the antibody with an organic derivatizing agent that is capable of reacting with selected side chains or the N- or C-terminal residues. Example covalent modifications of polypeptides are described in U.S. Pat. No. 5,534,615, specifically incorporated herein by reference. A preferred type of covalent modification of the antibody comprises linking the antibody to one of a variety of non-proteinaceous polymers, e.g., polyethylene glycol, polypropylene glycol, or polyoxyalkylenes, in the manner set forth in, e.g., U.S. Pat. Nos. 4,640,835; 4,496,689; 4,301,144; 4,670,417; 4,791,192 or 4,179,337.

### IX. Nucleic Acids, Vectors, Host Cells, and Recombinant Methods

The disclosure also provides isolated nucleic acids encoding an anti-α3β1 antibody, vectors and host cells comprising the nucleic acid, and recombinant techniques for the production of the antibody. A nucleic acid herein may include one or more subsequences, each referred to as a polynucleotide.

Provided herein are nucleic acids (e.g., isolated nucleic acids) comprising a nucleotide sequence that encodes an anti-α3β1 antibody, or fragment thereof. In some embodiments, a nucleic acid encodes an immunoglobulin heavy chain variable domain of an anti-α3β1 antibody provided herein. In some embodiments, a nucleic acid encodes an immunoglobulin light chain variable domain of an anti-α3β1 antibody provided herein. In some embodiments, a nucleic acid encodes an immunoglobulin heavy chain variable domain and an immunoglobulin light chain variable domain of an anti-α3β1 antibody provided herein. In some embodiments, a nucleic acid comprises a nucleotide sequence that encodes an amino acid sequence of any one of SEQ ID NOS: 1-37.

For recombinant production of an anti-α3β1 antibody, a nucleic acid encoding the anti-α3β1 antibody may be isolated and inserted into a replicable vector for further cloning (amplification of the DNA) or for expression. In certain instances, an anti-α3β1 antibody may be produced by homologous recombination. DNA encoding an anti-α3β1 antibody can be readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, and origin of replication, one or more marker genes, an enhancer element, a promoter, and a transcription termination sequence.

Suitable host cells for cloning or expressing DNA in vectors herein can be prokaryote, yeast, or higher eukaryote cells. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces. One preferred E. coli cloning host is E. coli 294 (ATCC 31,446), although other strains such as E. coli B, E. coli X₁₇₇₆ (ATCC 31,537), and E. coli W3110 (ATCC 27,325) can also be suitable. These examples are illustrative rather than limiting.

In addition to prokaryotes, eukaryotic microbes such as filamentous fungi or yeast are suitable cloning or expression hosts for anti-α3β1 antibody-encoding vectors. Saccharomyces cerevisiae, or common baker's yeast, is the most commonly used among lower eukaryotic host microorganisms. A number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of anti-α3β1 antibodies can also be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori (silk moth) have been identified. A variety of viral strains for transfection are publicly available, e.g., the L-1 variant of Autographa californica NPV and the Bm-5 strain of Bombyx mori NPV, and such viruses may be used as the virus herein according to the present technology, particularly for transfection of Spodoptera frugiperda cells. Plant cell cultures of cotton, corn, potato, soybean, petunia, tomato, and tobacco can also be utilized as hosts.

Suitable host cells for the expression of anti-α3β1 antibodies also may include vertebrate cells (e.g., mammalian cells). Vertebrate cells may be propagated in culture (tissue culture). Examples of useful mammalian host cell lines include monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse Sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells may be transformed with the above-described expression or cloning vectors for antibody production and cultured in conventional nutrient media modified as appropriate for inducing promoters, selecting transformants, or amplifying the genes encoding the desired sequences. Host cells used to produce antibodies provided herein may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, antibodies can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies that are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, Bakerbond ABX™ resin (J. T. Baker, Phillipsburg, N.J.) is useful for purification. Other techniques for protein purification, such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between, e.g., about 2.5-4.5, and may be performed at low salt concentrations (e.g., from about 0-0.2 5M salt).

### X. Pharmaceutical Formulations, Dosing, and Routes of Administration

The present disclosure provides anti-α3β1 antibodies and related compositions, which may be useful for elimination of α3β1-expressing pathogens from the body, for example, and for identification and quantification of the number of α3β1-expressing pathogens in biological samples, for example.

Anti-α3β1 antibodies may be formulated in a pharmaceutical composition that is useful for a variety of purposes, including the treatment of diseases or disorders. Pharmaceutical compositions comprising one or more anti-α3β1 antibodies may be administered using a pharmaceutical device to a patient in need thereof, and according to one embodiment of the technology, kits are provided that include such devices. Such devices and kits may be designed for routine administration, including self-administration, of the pharmaceutical compositions herein.

Therapeutic formulations of an antibody may be prepared for storage by mixing the agent or antibody having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, or polyethylene glycol (PEG).

Formulations herein may also contain more than one active compound as necessary for the particular indication being treated, preferably those with complementary activities that do not adversely affect each other. Such molecules are suitably present in combination in amounts that are effective for the purpose intended.

Formulations for in vivo administration generally are sterile. This may be accomplished for instance by filtration through sterile filtration membranes, for example.

Sustained-release preparations may be prepared. Suitable examples of sustained-release preparations include semipermeable matrices of solid hydrophobic polymers containing the agent/antibody, which matrices are in the form of shaped articles, e.g., films, or microcapsule. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and gamma ethyl-L-glutamate, non-degradable ethylene-vinyl acetate, degradable lactic acid-glycolic acid copolymers such as the Lupron Depot® (injectable microspheres composed of lactic acid-glycolic acid copolymer and leuprolide acetate), and poly-D-(−)-3-hydroxybutyric acid. While polymers such as such as ethylene-vinyl acetate and lactic acid-glycolic acid enable release of molecules for over 100 days, certain hydrogels release proteins for shorter time periods. When encapsulated agents/antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thiol-disulfide interchange, stabilization may be achieved by modifying sulfhydryl residues, lyophilizing from acidic solutions, controlling moisture content, using appropriate additives, and developing specific polymer matrix compositions.

For therapeutic applications, anti-α3β1 antibodies provided herein are administered to a mammal, e.g., a human, in a pharmaceutically acceptable dosage form such as those discussed above, including those that may be administered to a human intravenously as a bolus or by continuous infusion over a period of time, or by intramuscular, intraperitoneal, intra-cerebrospinal, subcutaneous, intra-articular, intrasynovial, intrathecal, oral, topical, or inhalation routes. For the prevention or treatment of disease, the appropriate dosage of agent or antibody will depend on the type of disease to be treated, as defined above, the severity and course of the disease, whether the antibody is administered for preventative or therapeutic purposes, previous therapy, the patient's clinical history and response to the antibody, and the discretion of the attending physician. The antibody is suitably administered to the patient at one time or over a series of treatments.

Depending on the type and severity of the disease, about 1 μg/kg to about 50 mg/kg (e.g., 0.1-20 mg/kg) of antibody may be an initial candidate dosage for administration to the patient, whether, for example, by one or more separate administrations, or by continuous infusion. A typical daily or weekly dosage might range from about 1 μg/kg to about 20 mg/kg or more, depending on the factors mentioned above. For repeated administrations over several days or longer, depending on the condition, the treatment is repeated until a desired suppression of disease symptoms occurs. However, other dosage regimens may be useful. The progress of this therapy is easily monitored by conventional techniques and assays, including, for example, radiographic imaging. Detection methods using the antibody to determine α3β1 levels in bodily fluids or tissues may be used in order to optimize patient exposure to the therapeutic antibody.

In some embodiments, a composition comprising an anti-α3β1 antibody herein can be administered as a monotherapy, and in some embodiments, the composition comprising the anti-α3β1 antibody can be administered as part of a combination therapy. In some cases, the effectiveness of the antibody in preventing or treating diseases may be improved by administering the antibody serially or in combination with another drug that is effective for those purposes, such as a chemotherapeutic drug for treatment of cancer or a microbial infection. In other cases, the anti-α3β1 antibody may serve to enhance or sensitize cells to chemotherapeutic treatment, thus permitting efficacy at lower doses and with lower toxicity. Certain combination therapies include, in addition to administration of the composition comprising an antibody that reduces the number of α3β1-expressing cells, delivering a second therapeutic regimen selected from the group consisting of a chemotherapeutic agent, radiation therapy, surgery, and a combination of any of the foregoing. Such other agents may be present in the composition being administered or may be administered separately. Also, the anti-α3β1 antibody may be suitably administered serially or in combination with the other agent or modality, e.g., chemotherapeutic drug or radiation for treatment of cancer, infection, and the like, or an immunosuppressive drug.

### XI. Methods

As described herein, integrin α3β1 is a key integrin on the surface of podocytes, which are cells in Bowman's capsule in the kidneys that wrap around capillaries of the glomerulus. Integrin α3β1 is essential for podocyte attachment to the outside of blood vessels to form a healthy glomerulus in the kidney. The antibodies described herein can act as allosteric agonist antibodies to integrin α3β1 and can enhance integrin-dependent ligand binding and cell adhesion, thus, preventing cell loss in the urine and protecting from loss in kidney function. Provided herein are also methods for treating diseases and/or conditions associated with a loss of podocytes in a subject in need thereof by administering to the subject an anti-α3β1 antibody described herein that binds to integrin α3β1 or a portion thereof (e.g., a sequence within a thigh-genu region of integrin α3β1). In some embodiments, diseases and/or conditions that are associated with a loss of podocytes can be diseases and/or conditions that are caused by a loss of podocytes (i.e., a loss in cell number and/or a loss in cell function). In some embodiments, diseases and/or conditions that are associated with a loss of podocytes can be diseases and/or conditions that affect the kidney, and thus, the loss of podocytes (i.e., a loss in cell number and/or a loss in cell function) can be a result or manifestation of the kidney disease and/or condition.

In some embodiments of the methods, the subject has a kidney disease that is associated with a loss of podocytes (i.e., a loss in cell number and/or a loss in cell function). A kidney disease can be a glomerular disease, such as a nephritic disease, a nephrotic disease, Alport's syndrome, or Focal Segmental Glomerulosclerosis (FSGS).

In some embodiments, the subject is undergoing, has undergone, or about to undergo a transplant procedure. In some embodiments, the transplant procedure is kidney transplant. In certain embodiments, the antibody is administered after the transplant procedure. In particular embodiments, the antibody is administered after the kidney transplant to protect, maintain, and/or improve kidney function and health.

In some embodiments, the disease or condition that is associated with a loss of podocytes in the subject is an autoimmune disease. In some embodiments, the autoimmune disease affects kidney function and/or kidney health. In some embodiments, the autoimmune disease is lupus nephritis. In some embodiments, the autoimmune disease is Goodpasture syndrome. In some embodiments, the autoimmune disease is anti-glomerular basement membrane (anti-GBM). In some embodiments, the autoimmune disease is ANCA-associated vasculitis and glomerulonephritis.

In other embodiments, the disease or condition that is associated with a loss of podocytes is cancer, in particular, cancers that affect kidney function and/or health. In certain embodiments, the cancer is kidney cancer. In certain embodiments, the cancer is renal cell carcinoma, urothelial carcinoma, kidney sarcoma, Wilms tumor, or lymphoma.

In some embodiments, the disease or condition that is associated with a loss of podocytes is an inflammation, in particular, inflammations that affect kidney function and/or health. In particular, the inflammation is glomerulonephritis. In some embodiments, the inflammation is membranoproliferative glomerulonephritis (MPGN), interstitial nephritis, IgA nephropathy (Berger's disease), pyelonephritis, lupus nephritis, or Wegener's granulomatosis.

The disclosure also features a method for identifying antibodies that bind to an integrin α3β1 or a portion thereof, comprising:
1) removing antibodies that bind against the β1 chain of the integrin α3β1 in the presence or absence of a ligand mimetic peptide and/or an antibody;
2) from the remaining antibodies from step 1), selecting for antibodies that bind to the integrin α3β1 in the presence or absence of a β1 agonist antibody;
3) counter selecting for antibodies that bind to the integrin α3β1 against immobilized β1 agonist antibodies or a ligand mimetic peptide alone; and
4) repeating steps 1), 2), and 3) above to enrich for antibodies that are integrin α3 allosteric agonists in the presence of cell surface-expressed integrin α3β1.

In some embodiments of the method, the ligand mimetic peptide is LXY2. In some embodiments of the method, step 1) and/or 3) is performed using human K562 cells that primarily express human α5β1 integrin and not over-express α3β1.

In some embodiments of the method, step 2) and/or 3) is performed using human K562 cells that over-express α3β1. In some embodiments, step 1) and/or 2) and/or 3) are performed in the presence of agents that block the ligand binding site or domain of the integrin, such as antibodies and ligands.

In some embodiments, integrin α3β1 is stabilized in a specific conformation by pre-complexing it with activating or inhibitory agents, such as activating antibody 9EG7 or TS2/16. In some other embodiments, integrin α3β1 is stabilized in a specific conformation by pre-complexing with agents that selectively bind the beta-chain of the integrin dimer.

We clear for β1 binders by clearing against “other” β1 integrins-either as recombinant proteins or using cell lines such as K562 that express α5β1 etc.

We prepare an α3β1 complex such that α3β1 is in a more “open” or “active” like conformation by pre-complexing α3β1 (recombinant or expressed on a cell line) with a β1 activating antibodies. This allows for easier identification of antibodies that bind active α3β1 or activate α3β1. We further increase the chance of identifying activating antibodies by adding a ligand or a ligand-mimetic to this complex. This prepared pre-complex is used for selecting activating antibodies.

We further force the identification of new antibodies to an allosteric stie by blocking the ligand binding face of the integrin complex using ligand mimetics or blocking antibodies.

### EXAMPLES

### Example 1—Identification of Antibody Fragments Using Phage Display

To find variable short chain fragments (scFv), a phage display library of naïve human scFv was run through a novel selection strategy to identify allosteric agonists. The strategy helps identify allosteric agonist binders, helps identify binders specific to one integrin chain over the other, and helps identify binders that increase ligand binding. Furthermore, the strategy relies on conformationally stabilized integrins (such as integrin α3β1 complexed with β1 activating antibody to stabilize the integrin in an “active” conformation) to help identify conformation-sensitive binders. Additionally, the strategy uses ligand blockers (such as integrin α3β1 complexed with ligand mimetic peptide LXY2 or ligand laminin, in the absence or presence of β1 activating antibody, to block the highly antigenic ligand binding pocket and the MIDAS site) to deliberately exclude binders to the ligand binding pocket. Here we used two selection methods, termed Selection 1 and Selection 2, to identify binders. Selection 1 utilized recombinant integrin and Selection 2 utilized cell surface expressed integrin. For Selection 1, a round of screening used three steps: pre-depletion, selection and counter selection. The pre-depletion step was used to remove binders against the β1 chain of the α3β1 dimer by using a negative selection, where phage binders are selected away from the screening pool, against immobilized recombinant human integrin α4β1. Additionally, a commercial β1 agonist antibody (TS2/16) and ligand mimetic peptide (LXY2) were included with the immobilized integrin α4β1 in some of the steps, to further remove any phage binders to those agents. The non-binding phages were used in the next step, selection step, for positive selection, where the phages were incubated with immobilized recombinant human or mouse integrin α3β1 in the presence or absence of β1 agonist antibodies (antibody clone TS2/16 for human α3β1 and antibody clone 9EG7 for mouse α3β1) and ligand mimetic peptide LXY2. The non binding phages were removed and discarded. Bound phages were eluted and used in the final step, where any binders to the β1 agonist antibodies and LXY2 were removed using a counter selection against immobilized β1 agonist antibodies and LXY2 alone. All non-binding phages were considered enriched as anti-integrin α3 allosteric binders. Furthermore, this process can be repeated for multiple rounds to further enrich phage clones of interest.

Next, the enriched phage pool from Selection 1 was optionally amplified and then taken through Selection 2 to enrich for anti-integrin α3 allosteric agonists that bind to cell surface expressed integrin, similar to the method described above. Here, a round of screening is structured as follows: 1) a pre-depletion step against K562 cells (primarily expressing human α5β1 integrin and not over-expressing α3β1), in the presence or absence of β1 agonist antibodies and ligand mimetic peptide LXY2; 2) a positive selection against human K562 cells expressing integrin α3β1, in the presence or absence of β1 agonist antibody TS2/16, and a counter selection against immobilized β1 agonist antibodies and LXY2 alone. Cell line generation is described in the methods.

The enriched phage pool from Selection 2 was optionally further amplified and plated using standard methods. 184 individual clones were identified and picked for clonal amplification step. Each clone was purified to yield a periplasmic extract (PE) solution containing soluble parental clone scFv. These extracts were tested via Direct Integrin ELISA (as described in the methods) against each of human «3β1, mouse α3β1, and human α4β1 in the presence or absence of LXY2. The extracts were then tested against human α3-expressing K562 and α3 non-expressing K562 by flow cytometry.

Following the characterization of the PE, the DNA of the variable domains of each scFv were sequenced. CDR sequences were assigned to each of the 184 clones, then aligned and clustered to eliminate duplicates. The 184 clones isolated from the selection yielded 25 unique sets of scFv CDR sequences. Sequencing data was combined with the assay data from PE ELISA and FACS to enable the selection of top hits.

### Example 2—Generation of Full Length IgG Antibodies

Using the dataset generated from the sequencing and characterization assays, the top five sequences were selected for reformatting. The heavy variable region DNA sequences of each scFv were appended to full-length human heavy constant IgG1 DNA (IGHC1 gene transcript) via gene synthesis and cloning. The light human Kappa variable regions and the Lambda variable region DNA sequences of each scFv were appended to full-length human light constant IGKC1 and IGLC1 DNA respectively, retaining their heavy/light pairing from the scFv level. The heavy and light DNA constructs were cloned into separate cloning vectors then shuttled into mammalian expression vectors.

Each of the five paired constructs were transfected and expressed in 10 mL of mammalian cells, then the antibodies were isolated using Protein A purification. Antibody samples were run on reducing and non-reducing SDS-PAGE as well as SEC-HPLC for quality control. Antibody samples were found at the expected molecular weights on the SDS-PAGE gels and SEC-HPLC peaks determined the samples are reasonably pure.

### Example 3—Validation of Antibody Binding Using ELISA

The five full-length IgG antibodies were termed Ab74 A100 through A104, or Ab74 for short. First, the binding of the five Ab74 to ECD was characterized by direct Integrin ELISA. Briefly, BSA, recombinant human integrin α3β1 ECD, recombinant human integrin α4β1 ECD, or recombinant mouse integrin α3β1 ECD were coated overnight on the plate then incubated with either each of the five Ab74 individually or an isotype human IgG1 antibody negative control or a commercial anti-human α3 antibody positive control. Binding was detected by incubating with anti-human IgG1 antibody Horse Radish Peroxidase (HRP) conjugate, then treating with fluorometric substrate, developing the reaction, and reading mean fluorescence intensity using a plate reader.

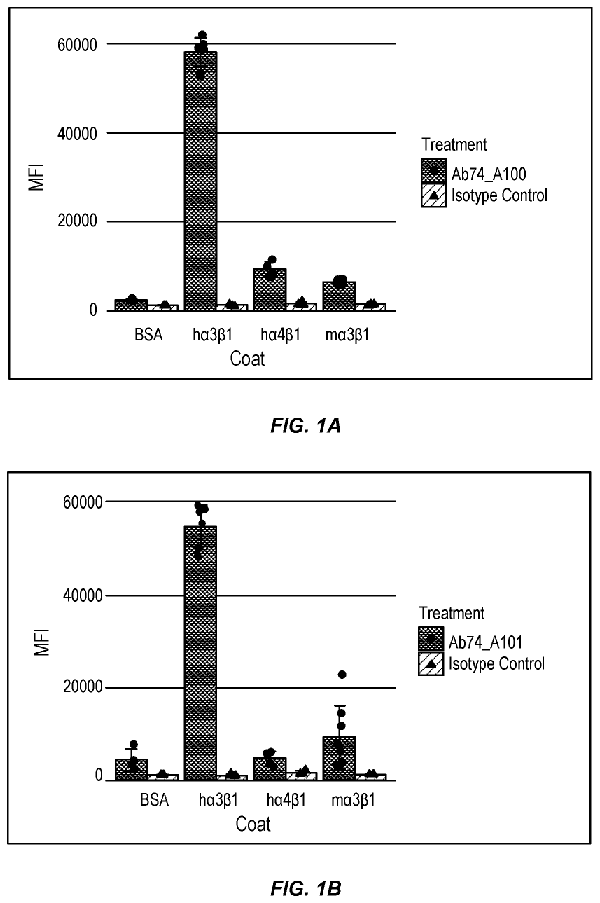

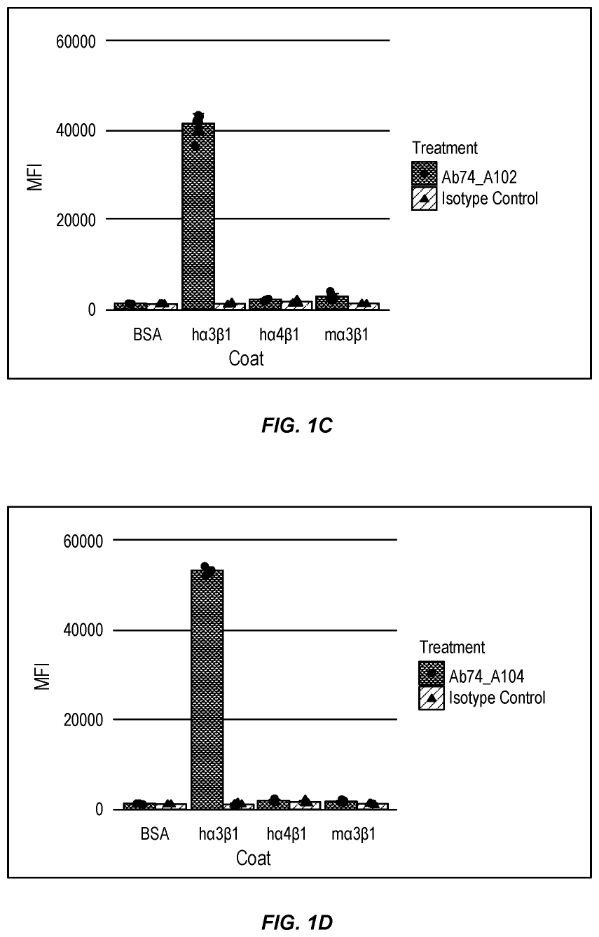

ELISA results show that the five Ab74 preferentially bind human α3β1 ECD over every other antigen coated on the plate (FIGS. 1A-1D). The isotype negative control and anti-α3 positive control yield expected negative and positive results, confirming the low background of the assay and the positive signal against human α3 respectively. Two of the five Ab74 antibodies demonstrate low binding towards mouse α3, and all five Ab74 show only background signal against coated BSA and human α4β1 ECD, confirming by ELISA that Ab74 bind human α3 ECD and not human β1 ECD.

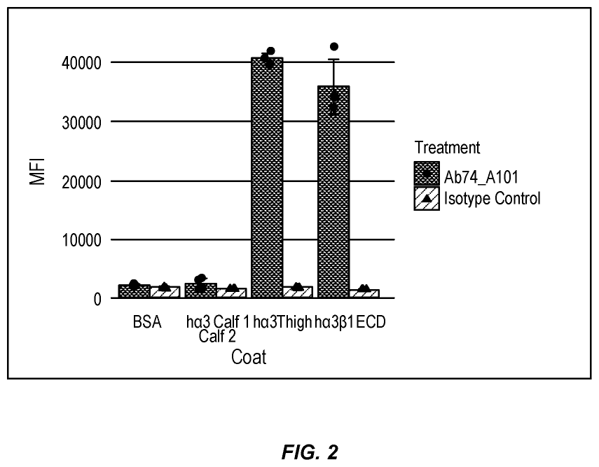

To further refine the binding site, individual human α3 domains were recombinantly expressed and purified in mammalian cells. Ab74 was tested against BSA, soluble human α3 Thigh-Genu, human α3 Calf1-Calf2, or human α3β1 ECD by Direct Integrin ELISA. The data suggests that Ab74 A101 binds the Thigh-Genu region in the sequence of the expressed construct (FIG. 2).

### Example 4—Validation of Antibody Binding Using Flow Cytometry-Based Assays

To validate antibody binding on cells, human or mouse integrin α3-expressing K562 cells were generated as described in the methods.

All full-length human IgG antibodies were detected by flow cytometry upon treatment of the K562 cells overexpressing either the human or mouse integrin α3 following both treatment and secondary antibody staining with an anti-human IgG1 antibody conjugated to a fluorophore. Results in Table 3 show high detection of Ab74 in both human and mouse integrin α3 expressing K562 cells when compared to negative control isotype antibody.

TABLE 3
Characterization of integrin agonist antibodies. Ab74 clones bind both human
and mouse integrin α3β1 as measured by ELISA and Flow Cytometry. α3β1 domain
mapping performed by Binding ELISA and Flow Cytometry using recombinant protein
domains and domain-swapped integrin-expressing cell lines, respectively.

| | Recombinant | | Human α3β1- | Mouse α3β1- |
|---|---|---|---|---|
| | Human α3β1 | | expressing cells | expressing cells |
| | Binding in | α3β1 Domain | Binding by | Binding by |
| Clone ID | ELISA | Mapping | FACS | FACS |
| Ab74 A100 | High | Thigh-Genu | High | High |
| Ab74 A101 | High | Thigh-Genu | High | High |
| Ab74 A102 | High | Thigh-Genu | High | High |
| Ab74 A103 | High | Thigh-Genu | High | High |
| Ab74 A104 | High | Thigh-Genu | High | High |

### Example 5—Increased Ligand Binding by Human Integrin α3β1 Expressing Cells in the Presence of Agonist Antibodies

To explore ligand binding agonization, human 3-expressing K562 were treated with the anti-α3 Ab74 or negative control isotype antibody or positive control commercial β1 agonist antibody TS2/16 in the presence of biotinylated ligand mimetic peptide LXY2 in a low-affinity Ca2+/Mg2+ buffer. Binding of LXY2 is detected by treating the cells with Streptavidin fluorophore conjugate and reading the cells in a flow cytometer. Negative and positive controls correctly show low-to-none and high ligand binding respectively in the low-affinity buffer. Results in Table 4 show Ab74 increases LXY2 binding in the low-affinity buffer compared to isotype antibody.

TABLE 4
Increased ligand binding by human integrin α3β1 expressing
cells in the presence of agonist antibodies. α3β1-expressing
K562 cells are incubated with α3β1 ligand mimetic LXY2-
biotin conjugate and either an integrin agonist antibody or
isotype control. Cells are then stained by Streptavidin-fluorophore
conjugate and measured by the flow cytometer.

| Clone ID | % Ligand Binding |
|---|---|
| Isotype Control | Low |
| Ab74 A100 | High |
| Ab74 A101 | High |
| Ab74 A102 | High |
| Ab74 A103 | High |
| Ab74 A104 | High |

### Example 6—Increased Ligand Binding by Mouse Integrin α3β1 Expressing Cells in the Presence of Agonist Antibodies

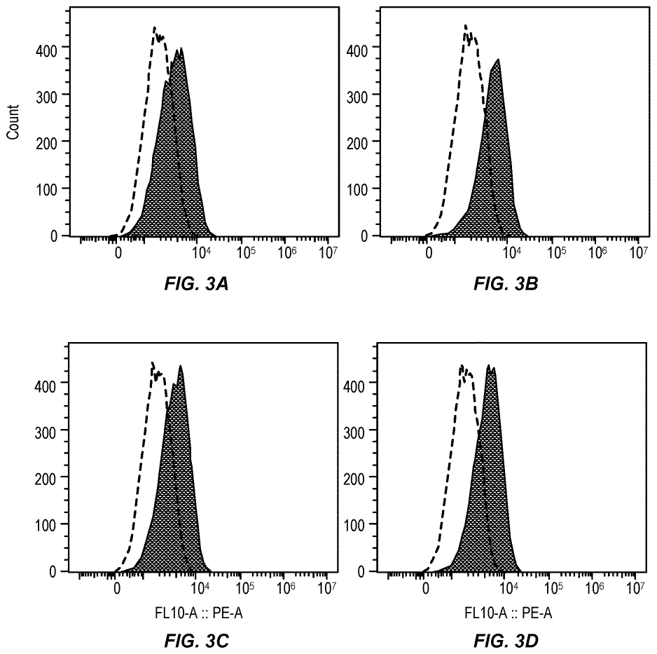

Cross-reactivity with mouse α3 was characterized by repeating the ligand experiments on the mouse α3-expressing K562. Briefly, the cells were treated with the anti-α3 Ab74 or negative control isotype antibody or positive control commercial β1 agonist antibody 9EG7 in the presence of biotinylated ligand mimetic peptide LXY2 in a low-affinity Ca2+/Mg2+ buffer. Binding of LXY2 is detected by treating the cells with Streptavidin fluorophore conjugate and reading the cells in a flow cytometer. Negative and positive controls correctly show low-to-none and high ligand binding respectively in the low-affinity buffer. Results in FIGS. 3A-3D show Ab74 increases LXY2 binding in the low-affinity buffer as well.

### Example 7—Reduced Cell Migration in the Presence of Integrin Agonist Antibodies

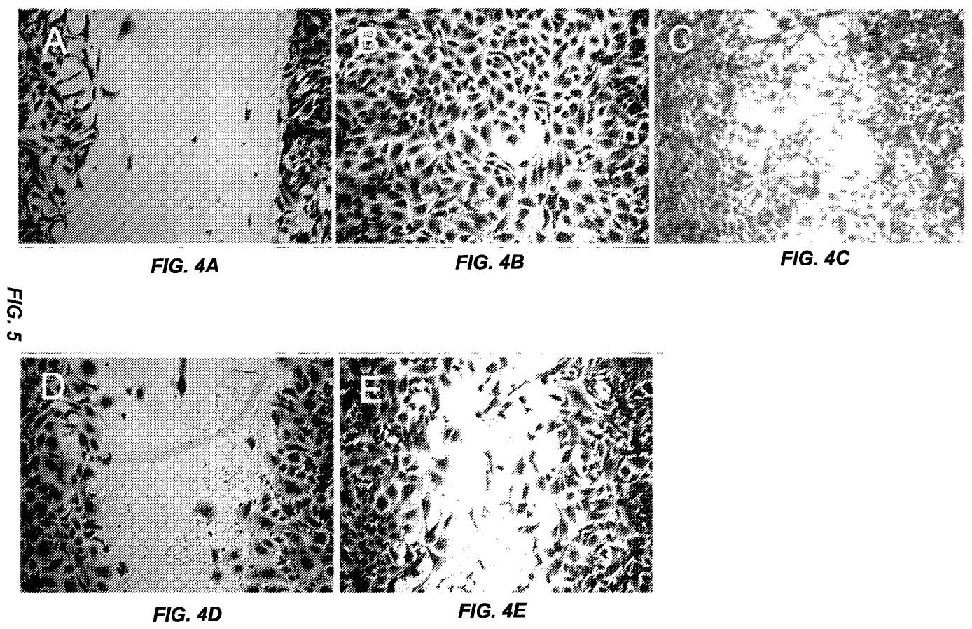

The adherent human ovarian cancer cell line SK-OV-3 expresses integrin α3β1 at high levels and mediates ligand binding to laminin-511. A tissue-culture treated 96-well plate was coated with integrin α3 ligand laminin-511 and incubated overnight. The next day, wells were plated with SK-OV-3 cells in serum-free media and cells were allowed to adhere. After 16 hours, a scratch (wound) is performed with a sterile plastic P200 pipette tip before adding anti-α3 Ab74 or negative control isotype antibody or positive control β1 agonist antibody in complete media. The change from serum-free media to media containing Fetal Bovine Serum (FBS) promotes cell migration and therefore wound closure via cell motility and migration. After 48 hours, the media was removed, the cells were fixed with 4% paraformaldehyde, and then stained with 0.2% Crystal violet. Results in FIGS. 4A-4E demonstrate inhibition of wound closure in wells treated with all Ab74 antibodies or positive control anti-βl agonist antibody when compared to isotype treated negative control or blocking anti-α3 which yielded wound closure.

### Example 8—Integrin Agonist Antibodies Target the Thigh-Genu Domain

For further refinement of the binding epitope of the antibodies, several α3 integrin domains were individually ‘exchanged’ with their homologous counterpart in the similar protein human integrin α7 (as described in the methods), which also contains the Thigh-Genu, Calf 1, and Calf 2 domains.

To this end, integrin DNA constructs were created where the Thigh-genu, Calf 1, and Calf 2 domains on integrin α7 replace the equivalent domains on the integrin α3 inside the mammalian expression plasmid. These three constructs were individually transfected into HEK-293 cells using Lipofectamine and allowed to expand for 48 hours under culture conditions. These cells were then treated with Ab74 or negative control isotype antibody or positive control commercial α3 antibody PIB5, before staining with a fluorophore conjugated anti-human IgG1 for detection in a flow cytometer.

Results in Table 5 show Ab74 detection in cells transfected with either the full-length integrin α3 DNA and construct with integrin α3 containing calf-2 of integrin α7 and construct with integrin α3 containing calf-1 of integrin α7 whereas little to no binding occurs in the integrin α3 construct replaced with Thigh-genu domain of integrin α7 suggesting that the five Ab74 antibodies recognize an epitope in the Thigh-Genu region of α3.

For the results in Table 5, for each integrin domain, a DNA construct was created where the respective domain is swapped out for its homologous integrin α7 counterpart. These are ITGA7 Thigh-genu, Calf 1, and Calf 2 inserted individually into α3, replacing the wild-type sequence. These DNA constructs are then cloned into mammalian expression vectors and individually transfected into mammalian cells over 48 hours, then incubated with activating human anti-α3 Abs. Antibody binding was detected by staining the cells with anti-hlgG1 antibody APC conjugate and reading on a flow cytometer.

TABLE 5
Epitope domain mapping of integrin agonist
antibodies by Flow Cytometry.

| | | | Chimeric | Chimeric | Chimeric |
|---|---|---|---|---|---|
| | | | α3β1 with | α3β1 | α3β1 |
| | Human | Human | α7 thigh- | with α7 | with α7 |
| | α3β1 | α7β1 | genu | Calf 1 | Calf 2 |
| | expressing | expressing | expressing | expressing | expressing |
| | cells, | cells, | cells, | cells, | cells, |
| Clone ID | binding | binding | binding | binding | binding |
| Ab74 | High | None | None | High | High |
| A100 | | | | | |
| Ab74 | High | None | None | High | High |
| A101 | | | | | |
| Ab74 | High | None | None | High | High |
| A102 | | | | | |
| Ab74 | High | None | None | High | High |
| A103 | | | | | |
| Ab74 | High | None | None | High | High |
| A104 | | | | | |

### Example 9—Methods

### Cell Culture and Transient Protein Expression in HEK293 Cell Line

HEK293 cells were cultured in serum-free CD medium (Sino Biological Cat #SMM 293-TI) until they reached optimum cell density. The expression vector was added to the cells with the presence of TF1 transfection reagent and serum-free feeder solution (Sino Biological Cat #M293-SUPI-100) was added to the culture at days 1, 3, and 5 upon transfection. Cells were harvested at culture day 7 and proceeded to protein purification.

Protein Purification from HEK293 Cells

Cells were removed via centrifugation and the culture supernatant was collected for protein purification.

Affinity purification: the column was equilibrated with loading buffer and the culture supernatant was loaded onto the column. The column was re-equilibrated, and the target protein was eluted by a gradient of buffer contain imidazole (Ni-affinity), or Glycine and NaCl (Protein A affinity or FLAG affinity). The protein is subjected to further buffer exchange to remove excessive imidazole or other salts. The protein solution was concentrated and protein concentration as well as purity was assayed by corresponding methods. The protein concentration was determined by UV while its purity was assayed by SDS-PAGE and western blotting.

### Cloning in Expression Vector

Restriction Site 1-Kozak sequence-Signal peptide-target protein-Stop codon-Restriction Site 2.

Signal peptide
(SEQ ID NO: 57)
N-term-MGWSCIILFLVATATGVHS-

### Protein Tags and Features

Some constructs have N-term FLAG, C-term 6×His with 3 Gly4Ser linkers and 3C protease cleavage site.
N-term FLAG Tag MDYKDDDDK (SEQ ID NO:58)
C-term 6× His Tag HHHHHH (SEQ ID NO:59)
(Gly4Ser)3 linker GGGGGGGGSGGGGS (SEQ ID NO:60)
PreScission protease (3C) LEVLFQGP (SEQ ID NO:61) cleavage site (LEVLFQ/GP (SEQ ID NO:61), (where “/” indicates the cleavage site))

### Domain Swapped Mammalian Expression Constructs

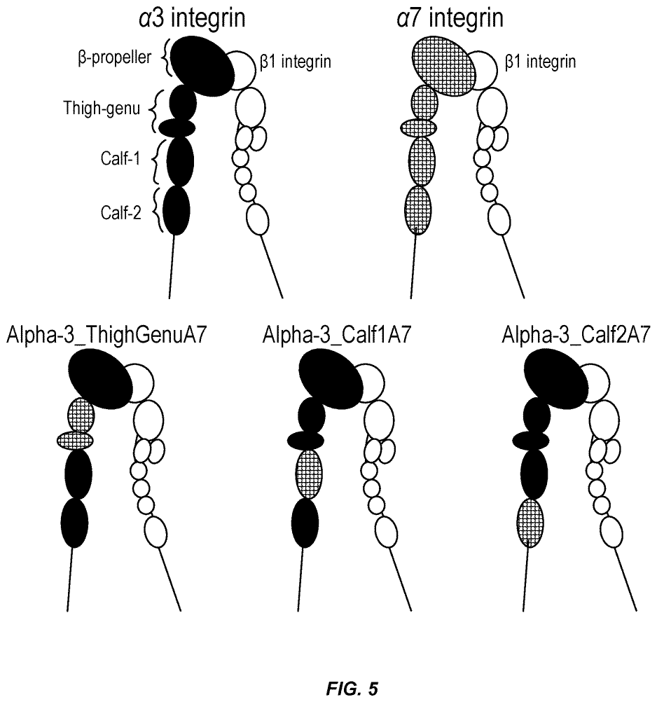

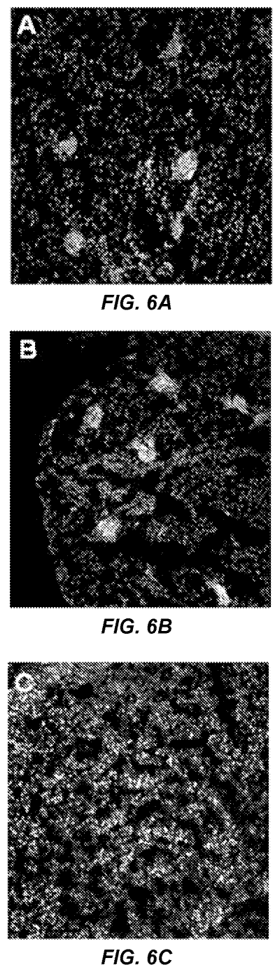

To test antibodies for domain specificity, individual domains from ITGA3 are replaced with the homologous respective domains from ITGA7 in the DNA sequence (FIG. 5). These are then inserted into a CMV driven mammalian expression vector (pCMV6-Neo) for transient transfection.

SEQ ID NO: 62:
Full-length human ITGA3 with swapped ITGA7 Thigh-Genu:
MGPGPSRAPRAPRLMLCALALMVAAGGCVVSAFNLDTRFLVVKEAGNPGSLFGYS
VALHRQTERQQRYLLLAGAPRELAVPDGYTNRTGAVYLCPLTAHKDDCERMNITV
KNDPGHHIIEDMWLGVTVASQGPAGRVLVCAHRYTQVLWSGSEDQRRMVGKCYV
RGNDLELDSSDDWQTYHNEMCNSNTDYLETGMCQLGTSGGFTQNTVYFGAPGAYN
WKGNSYMIQRKEWDLSEYSYKDPEDQGNLYIGYTMQVGSFILHPKNITIVTGAPRHR
HMGAVFLLSQEAGGDLRRRQVLEGSQVGAYFGSAIALADLNNDGWQDLLVGAPYY
FERKEEVGGAIYVFMNQAGTSFPAHPSLLLHGPSGSAFGLSVASIGDINQDGFQDIAV
GAPFEGLGKVYIYHSSSKGLLRQPQQVIHGEKLGLPGLATFGYSLSGQMDVDENFYP
DLLVGSLSDHIVLLRARPILHVSHEVSIAPRSIDLEQPNCAGGHSVCVDLRVCFSYIAV
PSSYSPTVALDYVLDADTDRRLRGQVPRVTFLSRNLEEPKHQASGTVWLKHQHDRV
CGDAMFQLQENVKDKLRAIVVTLSYSLQTPRLRRQAPGQGLPPVAPILNAHQPSTQR
AEIHFLKQGCGPDNKCESNLQMRAAFVSEQQQKLSRLQYSRDVRKLLLSINVTNTRT
SERSGEDAHEALLTLVVPPALLLSSVRPPGACQANETIFCELGNPFKRNQRMELLIAF
EVIGVTLHTRDLQVQLQLSTSSHQDNLWPMILTLLVDYTLQTSLSMVNHRLQSFFGG
TVMGESGMKTVEDVGSPLKYEFQVGPMGEGLVGLGTLVLGLEWPYEVSNGKWLL
YPTEITVHGNGSWPCRPPGDLINPLNLTLSDPGDRPSSPQRRRRQLDPGGGQGPPPVT
LAAAKKAKSETVLTCATGRAHCVWLECPIPDAPVVTNVTVKARVWNSTFIEDYRDF
DRVRVNGWATLFLRTSIPTINMENKTTWFSVDIDSELVEELPAEIELWLVLVAVGAG
LLLLGLIILLLWKCGFFKRARTRALYEAKRQKAEMKSQPSETERLTDDY*
SEQ ID NO: 63:
Full-length human ITGA3 with swapped ITGA7 Calf 1:
MGPGPSRAPRAPRLMLCALALMVAAGGCVVSAFNLDTRFLVVKEAGNPGSLFGYS
VALHRQTERQQRYLLLAGAPRELAVPDGYTNRTGAVYLCPLTAHKDDCERMNITV
KNDPGHHIIEDMWLGVTVASQGPAGRVLVCAHRYTQVLWSGSEDQRRMVGKCYV
RGNDLELDSSDDWQTYHNEMCNSNTDYLETGMCQLGTSGGFTQNTVYFGAPGAYN
WKGNSYMIQRKEWDLSEYSYKDPEDQGNLYIGYTMQVGSFILHPKNITIVTGAPRHR
HMGAVFLLSQEAGGDLRRRQVLEGSQVGAYFGSAIALADLNNDGWQDLLVGAPYY
FERKEEVGGAIYVFMNQAGTSFPAHPSLLLHGPSGSAFGLSVASIGDINQDGFQDIAV
GAPFEGLGKVYIYHSSSKGLLRQPQQVIHGEKLGLPGLATFGYSLSGQMDVDENFYP
DLLVGSLSDHIVLLRARPVINIVHKTLVPRPAVLDPALCTATSCVQVELCFAYNQSAG
NPNYRRNITLAYTLEADRDRRPPRLRFAGSESAVFHGFFSMPEMRCQKLELLLMDNL
RDKLRPIIISMNYSLPLRMPDRPRLGLRSLDAYPILNQAQALENHTEVQFQKECGPDN
KCQSNLQLVRARFCTRVSDTEFQPLPMDVDGTTALFALSGQPVIGLELMVTNLPSDP
AQPQADGDDAHEAQLLVMLPDSLHYSGVRALDPAEKPLCLSNENASHVECELGNP
MKRGAQVTFYLILSTSGISIETTELEVELLLATISEQELHPVSARARVFIELLQTSLSMV
NHRLQSFFGGTVMGESGMKTVEDVGSPLKYEFQVGPMGEGLVGLGTLVLGLEWPY
EVSNGKWLLYPTEITVHGNGSWPCRPPGDLINPLNLTLSDPGDRPSSPQRRRRQLDPG
GGQGPPPVTLAAAKKAKSETVLTCATGRAHCVWLECPIPDAPVVTNVTVKARVWN
STFIEDYRDFDRVRVNGWATLFLRTSIPTINMENKTTWFSVDIDSELVEELPAEIELWL
VLVAVGAGLLLLGLIILLLWKCGFFKRARTRALYEAKRQKAEMKSQPSETERLTDD
Y*
SEQ ID NO: 64:
Full-length human ITGA3, Calf 2 replaced with ITGA7 Calf 2:
MGPGPSRAPRAPRLMLCALALMVAAGGCVVSAFNLDTRFLVVKEAGNPGSLFGYS
VALHRQTERQQRYLLLAGAPRELAVPDGYTNRTGAVYLCPLTAHKDDCERMNITV
KNDPGHHIIEDMWLGVTVASQGPAGRVLVCAHRYTQVLWSGSEDQRRMVGKCYV
RGNDLELDSSDDWQTYHNEMCNSNTDYLETGMCQLGTSGGFTQNTVYFGAPGAYN
WKGNSYMIQRKEWDLSEYSYKDPEDQGNLYIGYTMQVGSFILHPKNITIVTGAPRHR
HMGAVFLLSQEAGGDLRRRQVLEGSQVGAYFGSAIALADLNNDGWQDLLVGAPYY
FERKEEVGGAIYVFMNQAGTSFPAHPSLLLHGPSGSAFGLSVASIGDINQDGFQDIAV
GAPFEGLGKVYIYHSSSKGLLRQPQQVIHGEKLGLPGLATFGYSLSGQMDVDENFYP
DLLVGSLSDHIVLLRARPVINIVHKTLVPRPAVLDPALCTATSCVQVELCFAYNQSAG
NPNYRRNITLAYTLEADRDRRPPRLRFAGSESAVFHGFFSMPEMRCQKLELLLMDNL
RDKLRPIIISMNYSLPLRMPDRPRLGLRSLDAYPILNQAQALENHTEVQFQKECGPDN
KCESNLQMRAAFVSEQQQKLSRLQYSRDVRKLLLSINVTNTRTSERSGEDAHEALLT
LVVPPALLLSSVRPPGACQANETIFCELGNPFKRNQRMELLIAFEVIGVTLHTRDLQV
QLQLSTSSHQDNLWPMILTLLVDYTPLSIAGMAIPQQLFFSGVVRGERAMQSERDVG
SKVKYEVTVSNQGQSLRTLGSAFLNIMWPHEIANGKWLLYPMQVELEGGQGPGQK
GLCSPRPNILHLDVDSRDRRRRELEPPEQQEPGERQEPSMSWWPVSSAEKKKNITLD
CARGTANCVVFSCPLYSFDRAAVLHVWGRLWNSTFLEEYSAVKSLEVIVRANITVKS
SIKNLMLRDASTVIPVMVYLDPMEELPAEIELWLVLVAVGAGLLLLGLIILLLWKCGF
FKRARTRALYEAKRQKAEMKSQPSETERLTDDY*

### Cell Line Generation

K562 cells from ATCC were transfected via electroporation with linearized expression plasmid containing human integrin α3 and kept under 0.5 mg/mL of G418 selection for 2 weeks. Cells were enriched for high integrin expression by Fluorescence Activated Cell Sorting (FACS) following staining protocol for anti-integrin α3 antibody staining using the commercial antibody PIB5 (Millipore Sigma, MA Waltham, MA, USA).

K562 mouse α3β1 and K562 cynomolgus monkey α3β1: K562 cells from ATCC were transfected via electroporation with linearized expression plasmid containing either mouse or cynomolgus monkey integrin α3 containing a C-terminus FLAG tag and kept under 0.8 mg/mL of puromycin selection for 2 weeks. Cells were enriched for high integrin expression by Fluorescence Activated Cell Sorting (FACS) following staining protocol for anti-FLAG antibody (Sino Biological, China).

### Cell Adhesion Assay, Fluorescent Reporter

Target integrin-expressing K562 cells expressing human integrin α3β1 were washed with TBS and 50,000 cells/well were transferred to ligand-coated wells of high binding clear 384-well microplates (Corning Incorporated, One Riverfront Plaza, NY, USA) in total 90 μL of assay buffer (HEPES 20 mM/2 mg/mL Glucose/140 mM NaCl containing 1 mM each of Ca₂₊ and Mg₂₊ denoted HEPES-CaMg). Plates were incubated at 37° C. in the presence of antibodies for 30 minutes. To induce detachment of non-adherent cells, plates were gently inverted and kept in that position for 45 minutes at room temperature. The plate were brought upright, and wells were quickly aspirated with an automated plate washer (Agilent Technologies, Santa Clara, CA, USA). Adherent cells were quantitated using CyQuantNF (Invitrogen, Waltham, MA, USA). For min-max normalization, a negative control assay buffer (HEPES buffer containing 10 mM EDTA denoted HEPES-EDTA) and a positive control assay buffer (TBS containing 1 mM of Mn₂₊ and 200 uM Ca₂₊ denoted TBS-Mn) are included in the plate.

### Cell Adhesion Assay, Automated Imaging

Target integrin-expressing K562 cells expressing human integrin α3β1 were washed with TBS and 50,000 cells/well were transferred to ligand-coated wells of high binding clear 384-well microplates (Corning Incorporated, One Riverfront Plaza, NY, USA) in total 90 μL of assay buffer (HEPES 20 mM/2 mg/mL Glucose/140 mM NaCl containing 1 mM each of Ca₂₊ and Mg₂₊ denoted HEPES-CaMg). Plates were incubated at 37° C. in the presence of antibodies for 30 minutes. To induce detachment of non-adherent cells, plates were gently inverted and kept in that position for 45 minutes at room temperature. The plate was brought upright, and wells are fixated using a stock solution of paraformaldehyde for a final concentration of 2% in inverted position for 10 minutes at room temperature The plate was brought upright and quickly aspirated with an automated plate washer (Agilent Technologies, Santa Clara, CA, USA). Adherent cells were quantitated using DAPI and an automated imaging system with a nucleus segmentation algorithm. For min-max normalization, a negative control assay buffer (TBS containing 10 mM EDTA denoted TBS-EDTA) and a positive control assay buffer (TBS containing 1 mM of Mn2+ and 200 μM Ca2+ denoted TBS-Mn) are included in the plate.

### Direct Integrin ELISA

High binding black 384-well microplates (Corning Incorporated, One Riverfront Plaza, NY, USA) were coated overnight with 30 μL TBS containing 3 μg/mL recombinant integrin at 4° C. The plates were flicked to remove any liquid and blocked by adding 90 μL of TBS containing 5% Bovine Serum Albumin (w/v), 0.05% Triton X-100 (v/v) and 0.025% (v/v) Pluronic F68 (Sigma-Aldrich, St. Louis, MO, USA) and incubating for 1 hour. After incubation, the plate was washed three times with 100 μL TBS using an automated plate washer (Agilent Technologies, Santa Clara, CA, USA). 30 μL assay buffer (TBS containing 0.05% Triton X-100 (v/v) and 0.025% (v/v) Pluronic F68, denoted TBS-T) were added to each well. 1 μL stock solution of test antibody was added to each corresponding well and the plate was centrifuged at 1000 g for 1 minute and incubated at room temperature for 1 hour. The plate was washed three times with 100 μL TBS using the automated washer. 30 μL of staining buffer (TBS-T containing a 1:2000 dilution of anti-IgG HRP conjugate) (Invitrogen, Waltham, MA, USA) was added to each well and incubated for 30 minutes. The plate was washed three times with 100 μL in an automated plate washer. 30 μL of substrate buffer is added (TBS containing 100 μM Amplex Red and 4 mM hydrogen peroxide) (Biotium, Fremont, CA, USA) and incubated for 30 minutes at room temperature. The plate was analyzed in a fluorescent microplate reader (Agilent Technologies, Santa Clara, CA, USA) at 563/587 nm.

### Integrin Sandwich ELISA

High binding black 384-well microplates (Corning Incorporated, One Riverfront Plaza, NY, USA) were coated overnight with 30 μL TBS containing 4 μg/mL anti-integrin antibody at 4° C. Assay buffer (TBS containing 0.05% Triton X-100 (v/v) and 0.025% (v/v) Pluronic F68 (Sigma-Aldrich, St. Louis, MO, USA), denoted TBS-T) was prepared ahead of the following steps. The plate was flicked to remove any liquid and blocked by adding 90 μL of TBS-T containing 5% Bovine Serum Albumin (w/v) (Sigma-Aldrich, St. Louis, MO, USA) and incubating for 1 hour. After incubation, the plate was washed three times with 100 μL TBS using an automated plate washer (Agilent Technologies, Santa Clara, CA, USA). 30 μL assay buffer (TBS-T containing 4 μg/mL tagged recombinant integrin) was added to each well. The plate was centrifuged at 1000 g for 1 minute and incubated at room temperature for 1 hour. The plate was washed three times with 100 μL TBS using the automated washer. 30 μL of staining buffer (TBS-T containing a 1:2000 dilution of anti-tag antibody HRP conjugate) (Invitrogen, Waltham, MA, USA) was added to each well and incubated for 30 minutes at room temperature. The plate was washed three times with 100 μL in an automated plate washer. 30 μL of substrate buffer (TBS containing 100 μM Amplex Red and 4 mM hydrogen peroxide) (Biotium, Fremont, CA, USA) was added to each well and developed for 30 minutes at room temperature. The plate was analyzed in a fluorescent microplate reader (Agilent Technologies, Santa Clara, CA, USA) at 563/587 nm.

### Recombinant Integrin Functional Assay (SoLISA), Integrin Detection

High binding black 384-well microplates (Corning Incorporated, One Riverfront Plaza, NY, USA) were coated overnight with 30 μL TBS containing 8 μg/mL ligand at 4° C. Assay buffer (TBS containing 0.05% Triton X-100 (v/v) and 0.025% (v/v) Pluronic F68 (Sigma-Aldrich, St. Louis, MO, USA), denoted TBS-T) was prepared ahead of the following steps. The plate was flicked to remove any liquid and blocked by adding 90 μL of TBS-T containing 5% Bovine Serum Albumin (w/v) (Sigma-Aldrich, St. Louis, MO, USA) and incubated for 1 hour. After incubation, the plate was washed three times with 100 μL TBS using an automated plate washer (Agilent Technologies, Santa Clara, CA, USA). 30 μL assay buffer (TBS-T containing 4 μg/mL tagged recombinant integrin and one of 1 mM Ca₂₊/1 mM Mg₂₊, 1 mM Mn₂₊/200 μM Ca₂₊, or 10 mM EDTA) was added to each well. 1 μL of agonist antibody (or isotype control) stock solution at an appropriate concentration was added to each well. The plate was centrifuged at 1000 g for 1 minute and incubated at room temperature for 3 hours. The plate was washed three times with 100 μL TBS using the automated washer. 30 μL of staining buffer (TBS-T containing a 1:2000 dilution of anti-tag antibody HRP conjugate) (Invitrogen, Waltham, MA, USA) was added to each well and incubated for 30 minutes at room temperature. The plate was washed three times with 100 μL in an automated plate washer. 30 μL of substrate buffer (TBS containing 100 μM Amplex Red and 4 mM hydrogen peroxide) (Biotium, Fremont, CA, USA) was added to each well and developed for 30 minutes at room temperature. The plate was analyzed in a fluorescent microplate reader (Agilent Technologies, Santa Clara, CA, USA) at 563/587 nm.

### Recombinant Integrin Functional Assay (SoLISA), Ligand Detection

High binding black 384-well microplates (Corning Incorporated, One Riverfront Plaza, NY, USA) were coated overnight with 30 μL TBS containing 4 μg/mL anti-tag antibody at 4° C. Assay buffer (TBS containing 0.05% Triton X-100 (v/v) and 0.025% (v/v) Pluronic F68 (Sigma-Aldrich, St. Louis, MO, USA), denoted TBS-T) were prepared ahead of the following steps. The plate was flicked to remove any liquid and blocked by adding 90 μL of TBS-T containing 5% Bovine Serum Albumin (w/v) then incubated for 1 hour. After incubation, the plate was washed three times with 100 μL TBS using an automated plate washer (Agilent Technologies, Santa Clara, CA, USA). 30 μL capture buffer (TBS-T containing 4 μg/mL tagged recombinant integrin) was added to each well. 30 μL assay buffer (TBS-T containing 8 μg/mL tagged recombinant ligand and one of 1 mM Ca₂₊/1 mM Mg₂₊, 1 mM Mn₂₊/200 μM Ca₂₊, or 10 mM EDTA) was added to each well. 1 μL of agonist antibody (or isotype control) stock solution at an appropriate concentration was added to each well. The plate was centrifuged at 1000 g for 1 minute and incubated at room temperature for 3 hours. The plate was washed three times with 100 μL TBS using the automated washer. 30 μL of staining buffer (TBS-T containing a 1:2000 dilution of anti-ligand-tag antibody HRP conjugate) (Invitrogen, Waltham, MA, USA) was added to each well and incubated for 30 minutes at room temperature. The plate was washed three times with 100 μL in an automated plate washer. 30 μL of substrate buffer (TBS containing 100 μM Amplex Red and 4 mM hydrogen peroxide) (Biotium, Fremont, CA, USA) was added to each well and developed for 30 minutes at room temperature. The plate was analyzed in a fluorescent microplate reader (Agilent Technologies, Santa Clara, CA, USA) at 563/587 nm.

### Recombinant Integrin Functional Assay (SoLISA), Antibody Detection

High binding black 384-well microplates (Corning Incorporated, One Riverfront Plaza, NY, USA) were coated overnight with 30 μL TBS containing 8 μg/mL ligand at 4° C. Assay buffer (TBS containing 0.05% Triton X-100 (v/v) and 0.025% (v/v) Pluronic F68 (Sigma-Aldrich, St. Louis, MO, USA), denoted TBS-T) is prepared ahead of the following steps. The plate was flicked to remove any liquid and blocked by adding 90 μL of TBS-T containing 5% Bovine Serum Albumin (w/v) (Sigma-Aldrich, St. Louis, MO, USA) then incubated for 1 hour. After incubation, the plate was washed three times with 100 μL TBS using an automated plate washer (Agilent Technologies, Santa Clara, CA, USA). 30 μL assay buffer (TBS-T containing 4 μg/mL tagged recombinant integrin and one of 1 mM Ca₂₊/1 mM Mg₂₊1 mM Mn₂₊/200 μM Ca₂₊, or 10 mM EDTA) was added to each well. 1 μL of agonist antibody (or isotype control) stock solution at an appropriate concentration was added to each well. The plate was centrifuged at 1000 g for 1 minute and incubated at room temperature for 3 hours. The plate was washed three times with 100 μL TBS using the automated washer. 30 μL of staining buffer (TBS-T containing a 1:2000 dilution of anti-IgG antibody HRP conjugate) (Invitrogen, Waltham, MA, USA) was added to each well and incubated for 30 minutes at room temperature. The plate was washed three times with 100 μL in an automated plate washer. 30 μL of substrate buffer (TBS containing 100 μM Amplex Red and 4 mM hydrogen peroxide) (Biotium, Fremont, CA, USA) was added to each well and developed for 30 minutes at room temperature. The plate was analyzed in a fluorescent microplate reader (Agilent Technologies, Santa Clara, CA, USA) at 563/587 nm.

### Soluble Ligand (Laminin 511) Binding Assay by Flow Cytometry

The day before the assay, integrin-expressing K562 cells in antibiotic-containing media were counted, washed with 10 mL of PBS, spun down and resuspended in complete media without positive selection antibiotic. These were cultured overnight at 37° C. with 5% CO₂. Laminin-511 E8 Fragment Fc (Acro Biosystems, Newark, DE, USA) were conjugated with anti-human IgG Alexa Fluor 647 conjugate (Jackson Immuno Research Labs, West Grove, PA, USA) in a 1:1.5 molar ratio (5 μg Laminin-511 E8 Fc per test group) incubated for 30 minutes at RT in the dark. The cells were centrifuged and resuspended in FACS buffer (PBS containing 2% Fetal Bovine Serum) (Summerlin Scientific, Hampton, NH, USA) at 10 million cells/mL. Human Fc block were added to cells (BD Biosciences, Franklin Lakes, NJ, USA) to a final 25 μg/mL and incubated for 15 minutes on ice. After incubation, add FACS buffer to dilute the concentration to 1 million cells/mL. Once cells are transferred to a V-bottom 96-well plate [Cat. No. 290-8116-01V], 40 μL of assay buffer containing one of 1 mM Ca₂₊/1 mM Mg₂₊ or 1 mM Mn₂₊/200 μM Ca₂₊ or 10 mM EDTA was added to each respective well followed by agonist antibody or isotype and incubated for 5 minutes at room temperature. Next, 5 μL of Laminin 511 E8 Fc/Ab-AF647 solution was added to each well and incubated for 25 minutes at room temperature. Plate was washed with 200 μL assay buffer. To detect binding of agonist antibody, cells were resuspended in 100 μL of detection buffer (Assay buffer containing 2.5-5.0 μg/mL of anti-IgG (BD Pharmigen) and incubated for 30 minutes in the dark at room temperature. Plate was washed with 200 μL assay buffer and pellet was resuspended in 100 μL freshly prepared fixation buffer (PBS containing 4% paraformaldehyde) [Cat No. AA47377-9M] and incubated on ice for 10 minutes. Finally, cells were resuspended PBS and cells were analyzed on a CytoFLEX Flow Cytometer (Beckman Coulter, Pasadena, CA, USA).

### Soluble Ligand (Ligand-Mimic) Binding Assay by Flow Cytometry

The day before the assay, integrin-expressing K562 cells in antibiotic-containing media were counted, washed with 10 mL of PBS, spun down and resuspended in complete media without positive selection antibiotic. These were cultured overnight at 37 C with 5% CO₂. The cells were centrifuged and resuspended in FACS buffer (PBS containing 2% Fetal Bovine Serum) (Summerlin Scientific, Hampton, NH, USA) at 10 million cells/mL. Human Fc block were added to cells (BD Biosciences, Franklin Lakes, NJ, USA) to a final 25 μg/mL and incubated for 15 minutes on ice. After incubation, add FACS buffer to dilute the concentration to 1 million cells/mL. Once cells are transferred to a V-bottom 96-well plate [Cat. No. 290-8116-01V], 40 μL of assay buffer containing one of 1 mM Ca₂₊/1 mM Mg₂₊ or 1 mM Mn₂₊/200 μM Ca₂₊ or 10 mM EDTA was added to each respective well followed by agonist antibody or isotype and incubated for 5 minutes at room temperature. Next, 5 μL of 10× biotinylated-ligand mimic was added to each well and incubated for 25 minutes at room temperature. Plate was washed with 200 μL assay buffer. To detect binding of agonist antibody, cells were resuspended in 100 μL of detection buffer (Assay buffer containing 2.5-5.0 μg/mL of anti-IgG (BD Pharmigen). Biotinylated ligand mimic was detected by fluorescently tagged streptavidin and incubated for 30 minutes in the dark at room temperature. Plate was washed with 200 μL assay buffer and pellet was resuspended in 100 μL freshly prepared fixation buffer (PBS containing 4% paraformaldehyde) [Cat No. AA47377-9M] and incubated on ice for 10 minutes. Finally, cells were resuspended PBS and cells were analyzed on a CytoFLEX Flow Cytometer (Beckman Coulter, Pasadena, CA, USA).

### Wound Healing/Scratch Assay

The day before, a tissue-culture treated, flat 96 well-plate were coated with 100 μL of 2.0 μg/mL of laminin-511 (iMatrix) prepared in 1×PBS under sterile conditions and placed at 4° C. overnight. The next day, coating solution was aspirated, and the plate was blocked with 100 μL of sterile 2% FBS in for 1-hour at room temperature. To detach cells to seed for scratch assay, SKOV3 cell layer was treated 0.25% Trypsin-EDTA and 30,000 cells were seeded per well in 100 μL of warm serum-free media. Plate was centrifuged at 500 g for 5 minutes to settle cells and incubated overnight in cell incubator. The next day, a vertical wound/scratch in the middle of the well was created using a sterile p200 tip. Agonist or isotype antibody treatment were prepared in warm complete media, and 100 μL of treatment were added per well. Lastly, Ca₂₊Mg₂₊ or Mn₂₊ were added to a final concentration of 0.5 mM in 10 μL per well. The wound healing process was observed at 24 hours for wound closure, when complete media treated only is near closing, the media was aspirated, and cell layer was washed with 200 μL of PBS and fixed with 4% of PFA at 4° C. for 10 minutes. 200 μL of 0.5% Crystal violet were added to each well and stained for 30 minutes at RT.

Transient Transfection of 293HEK with Integrin Alpha Subunit Chimeric DNA Constructs

293HEK cells were plated in a 6-well plate at 500,000 cells per well. The day of the transfection, complete media was aspirated, cell layer was washed with 2 mL of PBS then 800 μL of Opti-MEM was gently added to cells. Transfection agent was prepared with 2.5 μg of DNA and 3 μL of lipofectamine 2000 in 250 μL of Opti-MEM then incubated for 5 minutes at room temperature. Solution was dispensed dropwise into wells, incubated overnight followed by complete media change. Cells were analyzed for Ab74 binding by flow cytometry and detected by anti-human IgG1 antibody conjugated to a fluorophore.

Additional sequences
Human recombinant integrin α3β1:
ITGA3 sp|P26006|33-991; Protein sequence: 1034 aa
(SEQ ID NO: 65)
MGWSCIILFLVATATGVHSFNLDTRFLVVKEAGNPGSLFGYSVALHRQTERQQRYLL
LAGAPRELAVPDGYTNRTGAVYLCPLTAHKDDCERMNITVKNDPGHHIIEDMWLGV
TVASQGPAGRVLVCAHRYTQVLWSGSEDQRRMVGKCYVRGNDLELDSSDDWQTY
HNEMCNSNTDYLETGMCQLGTSGGFTQNTVYFGAPGAYNWKGNSYMIQRKEWDL
SEYSYKDPEDQGNLYIGYTMQVGSFILHPKNITIVTGAPRHRHMGAVFLLSQEAGGD
LRRRQVLEGSQVGAYFGSAIALADLNNDGWQDLLVGAPYYFERKEEVGGAIYVFM
NQAGTSFPAHPSLLLHGPSGSAFGLSVASIGDINQDGFQDIAVGAPFEGLGKVYIYHS
SSKGLLRQPQQVIHGEKLGLPGLATFGYSLSGQMDVDENFYPDLLVGSLSDHIVLLR
ARPVINIVHKTLVPRPAVLDPALCTATSCVQVELCFAYNQSAGNPNYRRNITLAYTLE
ADRDRRPPRLRFAGSESAVFHGFFSMPEMRCQKLELLLMDNLRDKLRPIIISMNYSLP
LRMPDRPRLGLRSLDAYPILNQAQALENHTEVQFQKECGPDNKCESNLQMRAAFVS
EQQQKLSRLQYSRDVRKLLLSINVTNTRTSERSGEDAHEALLTLVVPPALLLSSVRPP
GACQANETIFCELGNPFKRNQRMELLIAFEVIGVTLHTRDLQVQLQLSTSSHQDNLW
PMILTLLVDYTLQTSLSMVNHRLQSFFGGTVMGESGMKTVEDVGSPLKYEFQVGPM
GEGLVGLGTLVLGLEWPYEVSNGKWLLYPTEITVHGNGSWPCRPPGDLINPLNLTLS
DPGDRPSSPQRRRRQLDPGGGQGPPPVTLAAAKKAKSETVLTCATGRAHCVWLECPI
PDAPVVTNVTVKARVWNSTFIEDYRDFDRVRVNGWATLFLRTSIPTINMENKTTWFS
VDIDSELVEELPAEIEGTGGLLEVLFQGPGENAQLEKELQALEKENAQLEWELQALE
KELAQGGDYKDDDDK.
ITGB1 sp|P05556|21-728; Protein sequence: 781 aa
(SEQ ID NO: 66)
MGWSCIILFLVATATGVHSQTDENRCLKANAKSCGECIQAGPNCGWCTNSTFLQEG
MPTSARCDDLEALKKKGCPPDDIENPRGSKDIKKNKNVTNRSKGTAEKLKPEDITQI
QPQQLVLRLRSGEPQTFTLKFKRAEDYPIDLYYLMDLSYSMKDDLENVKSLGTDLM
NEMRRITSDFRIGFGSFVEKTVMPYISTTPAKLRNPCTSEQNCTSPFSYKNVLSLTNKG
EVFNELVGKQRISGNLDSPEGGFDAIMQVAVCGSLIGWRNVTRLLVFSTDAGFHFAG
DGKLGGIVLPNDGQCHLENNMYTMSHYYDYPSIAHLVQKLSENNIQTIFAVTEEFQP
VYKELKNLIPKSAVGTLSANSSNVIQLIIDAYNSLSSEVILENGKLSEGVTISYKSYCK
NGVNGTGENGRKCSNISIGDEVQFEISITSNKCPKKDSDSFKIRPLGFTEEVEVILQYIC
ECECQSEGIPESPKCHEGNGTFECGACRCNEGRVGRHCECSTDEVNSEDMDAYCRK
ENSSEICSNNGECVCGQCVCRKRDNTNEIYSGKFCECDNFNCDRSNGLICGGNGVCK
CRVCECNPNYTGSACDCSLDTSTCEASNGQICNGRGICECGVCKCTDPKFQGQTCEM
CQTCLGVCAEHKECVQCRAFNKGEKKDTCTQECSYFNITKVESRDKLPQPVQPDPVS
HCKEKDVDDCWFYFTYSVNGNNEVMVHVVENPECPTGPDDTSGLLEVLFQGPGKN
AQLKKKLQALKKKNAQLKWKLQALKKKLAQGGHHHHHH.
Human recombinant integrin α3β1 domain Calfl-Calf2:
(SEQ ID NO: 67)
ESNLQMRAAFVSEQQQKLSRLQYSRDVRKLLLSINVTNTRTSERSGEDAHEALLTLV
VPPALLLSSVRPPGACQANETIFCELGNPFKRNQRMELLIAFEVIGVTLHTRDLQVQL
QLSTSSHQDNLWPMILTLLVDYTLQTSLSMVNHRLQSFFGGTVMGESGMKTVEDVG
SPLKYEFQVGPMGEGLVGLGTLVLGLEWPYEVSNGKWLLYPTEITVHGNGSWPCRP
PGDLINPLNLTLSDPGDRPSSPQRRRRQLDPGGGQGPPPVTLAAAKKAKSETVLTCAT
GRAHCVWLECPIPDAPVVTNVTVKARVWNSTFIEDYRDFDRVRVNGWATLFLRTSI
PTINMENKTTWFSVDIDSELVEELPAEIEGTGGLLEVLFQGPGENHHHHHH.
Human recombinant integrin α3β1 domain Thigh:
(SEQ ID NO: 68)
MGWSCIILFLVATATGVHSMDYKDDDDKGGGGSGGGGSGGGGSLEVLFQGPLRAR
PVINIVHKTLVPRPAVLDPALCTATSCVQVELCFAYNQSAGNPNYRRNITLAYTLEA
DRDRRPPRLRFAGSESAVFHGFFSMPEMRCQKLELLLMDNLRDKLRPIIISMNYSLPL
RMPDRPRLGLRSLDAYPILNQAQALENHTEVQFQLEVLFQGPGGGGSGGGGSGGGG
SHHHHHH.
Mouse recombinant integrin α3β1:
Mouse α3 ECD sequence; Protein sequence: 1036aa
(SEQ ID NO: 69)
MGWSCIILFLVATATGVHSFNLDTRFLVVKEAVNPGSLFGYSVALHRQTERQQRYLL
LAGAPRDLAVGDDYTNRTGAVYLCPLTAHKDDCERMDISEKSDPDHHIIEDMWLGV
TVASQGPAGRVLVCAHRYTKVLWSGLEDQRRMVGKCYVRGNDLQLDPGDDWQTY
HNEMCNSNTDYLQTGMCQLGTSGGFTQNTVYFGAPGAYNWKGNSYMIQRKDWDL
SEYSYRGSEEQGNLYIGYTVQVGNAILHPTDIITVVTGAPRHQHMGAVFLLKQESGG
DLQRKQVLKGTQVGAYFGSAIALADLNNDGWQDLLVGAPYYFERKEEVGGAVYVF
MNQAGASFPDQPSLLLHGPSRSAFGISIASIGDINQDGFQDIAVGAPFEGLGKVYIYHS
SSGGLLRQPQQIIHGEKLGLPGLATFGYSLSGKMDVDENLYPDLLVGSLSDHIVLLRA
RPVINILHRTLVARPAVLDPALCTATSCVQVELCFAYNQSAGNPNYRRNITLAYTLEA
DRDRRPPRLRFARSQSSVFHGFFSMPETHCQTLELLLMDNVRDKLRPIVIAMNYSLPL
RMPDRLKLGLRSLDAYPVLNQAQAMENHTEVHFQKECGPDNKCDSNLQMRAAFLS
EQLQPLSRLQYSRDTKKLFLSINVTNSPSSQRAGEDAHEALLTLEVPSALLLSSVRPSG
TCQANNETILCELGNPFKRNQRMELLIAFEVIGVTLHTRDLPVLLQLSTSSHQDNLQP
VLLTLQVDYTLQASLSLMNHRLQSFFGGTVMGEAAMKTAEDVGSPLKYEFQVSPVG
DGLAALGTLVLGLEWPYEVTNGKWLLYPTEITIHSNGSWPCQPSGNLVNPLNLTLSD
PGVTPLSPQRRRRQLDPGGDQSSPPVTLAAAKKAKSETVLTCSNGRARCVWLECPLP
DTSNITNVTVKARVWNSTFIEDYKDFDRVRVDGWATLFLRTSIPTINMENKTTWFSV
DIDSELVEELPAEIEGTGGLLEVLFQGPGENAQLEKELQALEKENAQLEWELQALEK
ELAQGGDYKDDDDK.
Mouse β1 ECD Sequence; Protein sequence: 781aa
(SEQ ID NO: 70)
MGWSCIILFLVATATGVHSQTDKNRCLKANAKSCGECIQAGPNCGWCTNTTFLQEG
MPTSARCDDLEALKKKGCQPSDIENPRGSQTIKKNKNVTNRSKGMAEKLRPEDITQI
QPQQLLLKLRSGEPQKFTLKFKRAEDYPIDLYYLMDLSYSMKDDLENVKSLGTDLM
NEMRRITSDFRIGFGSFVEKTVMPYISTTP AKLRNPCTSEQNCTSPFSYKNVLSLTDRG
EFFNELVGQQRISGNLDSPEGGFDAIMQVAVCGSLIGWRNVTRLLVFSTDAGFHFAG
DGKLGGIVLPNDGQCHLENNVYTMSHYYDYPSIAHLVQKLSENNIQTIFAVTEEFQP
VYKELKNLIPKSAVGTLSGNSSNVIQLIIDAYNSLSSEVILENSKLPDGVTINYKSYCK
NGVNGTGENGRKCSNISIGDEVQFEISITANKCPNKESETIKIKPLGFTEEVEVVLQFIC
KCNCQSHGIPASPKCHEGNGTFECGACRCNEGRVGRHCECSTDEVNSEDMDAYCRK
ENSSEICSNNGECVCGQCVCRKRDNTNEIYSGKFCECDNFNCDRSNGLICGGNGVCR
CRVCECYPNYTGSACDCSLDTGPCLASNGQICNGRGICECGACKCTDPKFQGPTCET
CQTCLGVCAEHKECVQCRAFNKGEKKDTCAQECSHFNLTKVESREKLPQPVQVDPV
THCKEKDIDDCWFYFTYSVNGNNEAIVHVVETPDCPTGPDDTSGLLEVLFQGPGKN
AQLKKKLQALKKKNAQLKWKLQALKKKLAQGGHHHHHH.
Mouse recombinant integrin α3β1 domain Calf1-Calf2:
(SEQ ID NO: 71)
MGWSCIILFLVATATGVHSDSNLQMRAAFLSEQLQPLSRLQYSRDTKKLFLSINVTNS
PSSQRAGEDAHEALLTLEVPSALLLSSVRPSGTCQANNETILCELGNPFKRNQRMELL
IAFEVIGVTLHTRDLPVLLQLSTSSHQDNLQPVLLTLQVDYTLQASLSLMNHRLQSFF
GGTVMGEAAMKTAEDVGSPLKYEFQVSPVGDGLAALGTLVLGLEWPYEVTNGKW
LLYPTEITIHSNGSWPCQPSGNLVNPLNLTLSDPGVTPLSPQRRRRQLDPGGDQSSPPV
TLAAAKKAKSETVLTCSNGRARCVWLECPLPDTSNITNVTVKARVWNSTFIEDYKD
FDRVRVDGWATLFLRTSIPTINMENKTTWFSVDIDSELVEELPAEIEGENHHHHHH.

The above examples are provided to illustrate the disclosure but not to limit its scope. Other variants of the disclosure will be readily apparent to one of ordinary skill in the art and are encompassed by the appended claims. All publications, databases, internet sources, patents, patent applications, and accession numbers cited herein are hereby incorporated by reference in their entireties for all purposes.

1. An isolated antibody that binds to an integrin α3β1 or a portion thereof, comprising:
(1) a heavy chain complementarity-determining region 1 (CDR H1) comprising the sequence of X₁X₂SGX₃TFX₄X₅YX₆X₇X₈ (SEQ ID NO:38), wherein X₁ is A or K; X₂ is A or T; X₃ is F, G, or Y; X₄ is S or T; X₅ is S or N; X₆ is G, S, or A; X₇ is M or I; and X₈ is H, N, or S;
(2) a CDR H2 comprising a sequence having up to two amino acid substitutions relative to the sequence of GISGSADTTY (SEQ ID NO:6), SISSSSSYIY (SEQ ID NO:9), or GIIPIFGTAN (SEQ ID NO:10), or the sequence of WISAX₁NGNX₂N (SEQ ID NO:39), wherein X₁ is Y or N; and X₂ is T or S;
(3) a CDR H3 comprising a sequence having up to two amino acid substitutions relative to the sequence of VRDDIQLRD (SEQ ID NO:11) or AREFPGWYFDY (SEQ ID NO:13), or a sequence having up to four amino acid substitutions relative to the sequence of
(SEQ ID NO: 12)
ARDYSGSWYPSNGPALDY,
(SEQ ID NO: 14)
AREYYDFWSGYPSGYAFDI,
or
(SEQ ID NO: 15)
ARGVPSGSGYYLGLDY;
(4) a light chain complementarity-determining region 1 (CDR L1) comprising the sequence of X₁ASQX₂ISX₃YLN (SEQ ID NO:40), wherein X₁ is Q or A; X₂ is D or Y; and X₃ is N or S, or a sequence having up to three amino acid substitutions relative to the sequence of
(SEQ ID NO: 23)
QGDSLRSYYAS
or
(SEQ ID NO: 24)
SGSSSNIGSNYVY;
(5) a CDR L2 comprising a sequence having up to one amino acid substitution relative to the sequence of YDASNLET (SEQ ID NO:25), or the sequence of YX₁X₂NX₃RPS (SEQ ID NO:41), wherein X₁ is G or R; X₂ is K or N; and X₃ is N or Q; and
(6) a CDR L3 comprising the sequence of X₁QX₂YX₃X₄PX₅T (SEQ ID NO:42), wherein X₁ is L or Q; X₂ is D or S; X₃ is N, S, or R; X₄ is Y or T; and X₅ is L or P, or a sequence having up to two amino acid substitutions relative to the sequence of NSRDSSGNHWV (SEQ ID NO:31) or AAWDDSLSGPV (SEQ ID NO:32).

2. The isolated antibody of claim 1, wherein:
(1) the CDR H1 comprises a sequence of any one of AASGFTFSSYGMH (SEQ ID NO:1), KASGYTFTSYGIS (SEQ ID NO:2), KTSGFTFTNYGIS (SEQ ID NO:3), AASGFTFSSYSMN (SEQ ID NO:4), and KASGGTFSSYAIN (SEQ ID NO:5);
(2) the CDR H2 comprises a sequence of any one of GISGSADTTY (SEQ ID NO:6), WISAYNGNTN (SEQ ID NO:7), WISANNGNSN (SEQ ID NO:8), SISSSSSYIY (SEQ ID NO:9), and GIIPIFGTAN (SEQ ID NO:10);
(3) the CDR H3 comprises a sequence of any one of VRDDIQLRD (SEQ ID NO:11), ARDYSGSWYPSNGPALDY (SEQ ID NO:12), AREFPGWYFDY (SEQ ID NO:13), AREYYDFWSGYPSGYAFDI (SEQ ID NO:14), and ARGVPSGSGYYLGLDY (SEQ ID NO:15);
(4) the CDR L1 comprises the sequence of any one of QASQDISNYLN (SEQ ID NO:21), RASQYISSYLN (SEQ ID NO:22), QGDSLRSYYAS (SEQ ID NO:23), and SGSSSNIGSNYVY (SEQ ID NO:24);
(5) the CDR L2 comprises a sequence of any one of YDASNLET (SEQ ID NO:25), YGKNNRPS (SEQ ID NO:26), and YRNNQRPS (SEQ ID NO:27); and
(6) the CDR L3 comprises a sequence of any one of LQDYNYPLT (SEQ ID NO:28), LQDYSYPLT (SEQ ID NO:29), QQSYRTPPT (SEQ ID NO:30), NSRDSSGNHWV (SEQ ID NO:31), and AAWDDSLSGPV (SEQ ID NO:32).

3. The isolated antibody of claim 1, wherein
(a) the CDR H1 comprises the sequence of SEQ ID NO:1; the CDR H2 comprises the sequence of SEQ ID NO:6; and the CDR H3 comprises the sequence of SEQ ID NO:11;
(b) the CDR H1 comprises the sequence of SEQ ID NO:2; the CDR H2 comprises the sequence of SEQ ID NO:7; and the CDR H3 comprises the sequence of SEQ ID NO:12;
(c) the CDR H1 comprises the sequence of SEQ ID NO:3; the CDR H2 comprises the sequence of SEQ ID NO:8; and the CDR H3 comprises the sequence of SEQ ID NO:13;
(d) the CDR H1 comprises the sequence of SEQ ID NO:4; the CDR H2 comprises the sequence of SEQ ID NO:9; and the CDR H3 comprises the sequence of SEQ ID NO:14;
(e) the CDR H1 comprises the sequence of SEQ ID NO:5; the CDR H2 comprises the sequence of SEQ ID NO:10; and the CDR H3 comprises the sequence of SEQ ID NO:15.

4.-7. (canceled)

8. The isolated antibody of claim 1, wherein
(a) the CDR L1 comprises the sequence of SEQ ID NO:21; the CDR L2 comprises the sequence of SEQ ID NO:25; and the CDR L3 comprises the sequence of SEQ ID NO:28;
(b) the CDR L1 comprises the sequence of SEQ ID NO:22; the CDR L2 comprises the sequence of SEQ ID NO:25; and the CDR L3 comprises the sequence of SEQ ID NO:29;
(c) the CDR L1 comprises the sequence of SEQ ID NO:21; the CDR L2 comprises the sequence of SEQ ID NO:25; and the CDR L3 comprises the sequence of SEQ ID NO:30;
(d) the CDR L1 comprises the sequence of SEQ ID NO:23; the CDR L2 comprises the sequence of SEQ ID NO:26; and the CDR L3 comprises the sequence of SEQ ID NO:31
(e) the CDR L1 comprises the sequence of SEQ ID NO:24; the CDR L2 comprises the sequence of SEQ ID NO:27; and the CDR L3 comprises the sequence of SEQ ID NO:32.

9.-12. (canceled)

13. The isolated antibody of claim 1, wherein the antibody comprises a heavy chain variable region having at least 90% identity to a sequence of any one of SEQ ID NOS: 16-20; and/or a light chain variable region having at least 90% identity to a sequence of any one of SEQ ID NOS: 33-37.

14. (canceled)

15. The isolated antibody of claim 1, wherein the antibody comprises a HCDR1 having the sequence of SEQ ID NO:1, a HCDR2 having the sequence of SEQ ID NO:6, a HCDR3 having the sequence of SEQ ID NO:11, a LCDR1 having the sequence of SEQ ID NO:21, a LCDR2 having the sequence of SEQ ID NO:25, and a LCDR3 having the sequence of SEQ ID NO:28.

16. The isolated antibody of claim 15, wherein the antibody comprises a heavy chain variable region having at least 90% identity to the sequence of SEQ ID NO:16; and/or a light chain variable region having at least 90% identity to the sequence of SEQ ID NO:33.

17. (canceled)

18. The isolated antibody of claim 1, wherein the antibody comprises a HCDR1 having the sequence of SEQ ID NO:2, a HCDR2 having the sequence of SEQ ID NO:7, a HCDR3 having the sequence of SEQ ID NO:12, a LCDR1 having the sequence of SEQ ID NO:22, a LCDR2 having the sequence of SEQ ID NO:25, and a LCDR3 having the sequence of SEQ ID NO:29.

19. The isolated antibody of claim 18, wherein the antibody comprises a heavy chain variable region having at least 90% identity to the sequence of SEQ ID NO:17; and/or a light chain variable region having at least 90% identity to the sequence of SEQ ID NO:34.

20. (canceled)

21. The isolated antibody of claim 1, wherein the antibody comprises a HCDR1 having the sequence of SEQ ID NO:3, a HCDR2 having the sequence of SEQ ID NO:8, a HCDR3 having the sequence of SEQ ID NO:13, a LCDR1 having the sequence of SEQ ID NO:21, a LCDR2 having the sequence of SEQ ID NO:25, and a LCDR3 having the sequence of SEQ ID NO:30.

22. The isolated antibody of claim 21, wherein the antibody comprises a heavy chain variable region having at least 90% identity to the sequence of SEQ ID NO:18; and/or comprises a light chain variable region having at least 90% identity to the sequence of SEQ ID NO:35.

23. (canceled)

24. The isolated antibody of claim 1, wherein the antibody comprises a HCDR1 having the sequence of SEQ ID NO:4, a HCDR2 having the sequence of SEQ ID NO:9, a HCDR3 having the sequence of SEQ ID NO:14, a LCDR1 having the sequence of SEQ ID NO:23, a LCDR2 having the sequence of SEQ ID NO:26, and a LCDR3 having the sequence of SEQ ID NO:31.

25. The isolated antibody of claim 24, wherein the antibody comprises a heavy chain variable region having at least 90% identity to the sequence of SEQ ID NO:19; and/or a light chain variable region having at least 90% identity to the sequence of SEQ ID NO:36.

26. (canceled)

27. The isolated antibody of claim 1, wherein the antibody comprises a HCDR1 having the sequence of SEQ ID NO:5, a HCDR2 having the sequence of SEQ ID NO:10, a HCDR3 having the sequence of SEQ ID NO:15, a LCDR1 having the sequence of SEQ ID NO:24, a LCDR2 having the sequence of SEQ ID NO:27, and a LCDR3 having the sequence of SEQ ID NO:32.

28. The isolated antibody of claim 27, wherein the antibody comprises a heavy chain variable region having at least 90% identity to the sequence of SEQ ID NO:20; and/or a light chain variable region having at least 90% identity to the sequence of SEQ ID NO:37.

29. (canceled)

30. The isolated antibody of claim 1, wherein the antibody comprises an Fc polypeptide having at least 90% identity to a sequence of SEQ ID NO:43.

31. The isolated antibody of claim 1, wherein the antibody binds to a cell expressing integrin α3β1 or a portion thereof.

32. The isolated antibody of claim 31, wherein the cell is a podocyte or a neutrophil.

33. The isolated antibody of claim 1, wherein the antibody binds to the α3 portion of the integrin α3β1.

34. The isolated antibody of claim 33, wherein the antibody binds to a sequence within a thigh-genu region in the α3 portion.

35. The isolated antibody of claim 1, wherein the antibody binds to the sequence of SEQ ID NO:44 or a sequence within the sequence of SEQ ID NO:44.

36. The isolated antibody of claim 1, wherein the antibody is a monoclonal antibody, a humanized antibody, a full-length antibody, a Fab, a Fab′, a F(ab′)2, an Fv, a single chain Fv (scFv) antibody, or a bispecific antibody.

37.-39. (canceled)

40. An isolated nucleic acid encoding the isolated antibody of claim 1.

41. An expression vector comprising the nucleic acid of claim 40.

42. An isolated host cell comprising the vector of claim 41.

43. A pharmaceutical composition comprising the isolated antibody of claim 1 and a pharmaceutically acceptable carrier.

44. A method for treating a disease or condition associated with a loss of podocytes in a subject in need thereof, comprising administering to the subject an isolated antibody of claim 1.

45.-48. (canceled)

49. A method for identifying antibodies that bind to an integrin α3β1 or a portion thereof, comprising:
1) removing antibodies that bind against the β1 chain of the integrin α3β1 in the presence or absence of a ligand mimetic peptide and/or an antibody;
2) from the remaining antibodies from step 1), selecting for antibodies that bind to the integrin α3β1 in the presence or absence of a β1 agonist antibody;
3) counter selecting for antibodies that bind to the integrin α3β1 against immobilized β1 agonist antibodies or a ligand mimetic peptide alone; and
4) repeating steps 1), 2), and 3) above to enrich for antibodies that are integrin α3 allosteric agonists in the presence of cell surface-expressed integrin α3β1.

50.-51. (canceled)


SEQUENCE LISTING (71 sequences)

SEQ ID NO: 1 - protein, 13 aa, synthetic construct
AASGFTFSSYGMH

SEQ ID NO: 2 - protein, 13 aa, synthetic construct
KASGYTFTSYGIS

SEQ ID NO: 3 - protein, 13 aa, synthetic construct
KTSGFTFTNYGIS

SEQ ID NO: 4 - protein, 13 aa, synthetic construct
AASGFTFSSYSMN

SEQ ID NO: 5 - protein, 13 aa, synthetic construct
KASGGTFSSYAIN

SEQ ID NO: 6 - protein, 10 aa, synthetic construct
GISGSADTTY

SEQ ID NO: 7 - protein, 10 aa, synthetic construct
WISAYNGNTN

SEQ ID NO: 8 - protein, 10 aa, synthetic construct
WISANNGNSN

SEQ ID NO: 9 - protein, 10 aa, synthetic construct
SISSSSSYIY

SEQ ID NO: 10 - protein, 10 aa, synthetic construct
GIIPIFGTAN

SEQ ID NO: 11 - protein, 9 aa, synthetic construct
VRDDIQLRD

SEQ ID NO: 12 - protein, 18 aa, synthetic construct
ARDYSGSWYPSNGPALDY

SEQ ID NO: 13 - protein, 11 aa, synthetic construct
AREFPGWYFDY

SEQ ID NO: 14 - protein, 19 aa, synthetic construct
AREYYDFWSGYPSGYAFDI

SEQ ID NO: 15 - protein, 16 aa, synthetic construct
ARGVPSGSGYYLGLDY

SEQ ID NO: 16 - protein, 116 aa, synthetic construct
EVQLLESGGGVVQPGRSLRLSCAASGFTFSSYGMHWVRQAPGKGLEWLSGISGSADTTYYADSVKGRFTISRDNSKNTLYLQMTSLRAEDTAVYYCVRDDIQLRDWGQGTLVTVSS

SEQ ID NO: 17 - protein, 125 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDYSGSWYPSNGPALDYWGQGTMVTVSS

SEQ ID NO: 18 - protein, 118 aa, synthetic construct
EVQLVQSGAEVKKPGASVKVSCKTSGFTFTNYGISWVRQAPGQGLEWMGWISANNGNSNYAQDHQGRVTMTTDTSTSTAYMELRSLRSDDTAVYYCAREFPGWYFDYWGQGTLVTVSS

SEQ ID NO: 19 - protein, 126 aa, synthetic construct
EVQLVESGGGLVQPGGSLRLSCAASGFTFSSYSMNWVRQAPGKGLEWVSSISSSSSYIYYADSVKGRFTISRDNSKNTVYLQMNSLRAEDTAVYYCAREYYDFWSGYPSGYAFDIWGQGTLVTVSS

SEQ ID NO: 20 - protein, 123 aa, synthetic construct
QVQLVQSGAEVKKPGSSVKVSCKASGGTFSSYAINWVRQAPGQGLEWMGGIIPIFGTANYAQKFQGRVTITADKSTSTAYMELSSLRSEDTAVYYCARGVPSGSGYYLGLDYWGQGTMVTVSS

SEQ ID NO: 21 - protein, 11 aa, synthetic construct
QASQDISNYLN

SEQ ID NO: 22 - protein, 11 aa, synthetic construct
RASQYISSYLN

SEQ ID NO: 23 - protein, 11 aa, synthetic construct
QGDSLRSYYAS

SEQ ID NO: 24 - protein, 13 aa, synthetic construct
SGSSSNIGSNYVY

SEQ ID NO: 25 - protein, 8 aa, synthetic construct
YDASNLET

SEQ ID NO: 26 - protein, 8 aa, synthetic construct
YGKNNRPS

SEQ ID NO: 27 - protein, 8 aa, synthetic construct
YRNNQRPS

SEQ ID NO: 28 - protein, 9 aa, synthetic construct
LQDYNYPLT

SEQ ID NO: 29 - protein, 9 aa, synthetic construct
LQDYSYPLT

SEQ ID NO: 30 - protein, 9 aa, synthetic construct
QQSYRTPPT

SEQ ID NO: 31 - protein, 11 aa, synthetic construct
NSRDSSGNHWV

SEQ ID NO: 32 - protein, 11 aa, synthetic construct
AAWDDSLSGPV

SEQ ID NO: 33 - protein, 107 aa, synthetic construct
DIQMTQSPSSLSASVGDRVTITCQASQDISNYLNWYQQKPGKAPKLLIYDASNLETGVPSRFSGSGSGTDFALTISSLQPEDFATYYCLQDYNYPLTFGGGTKVDIK

SEQ ID NO: 34 - protein, 107 aa, synthetic construct
DIQMTQSPSSLSASVGDRVTITCRASQYISSYLNWYQQKPGKAPKLLIYDASNLETGVPSRFSGSGSGTDFTFTISSLQPEDIATYYCLQDYSYPLTFGGGIKVDIK

SEQ ID NO: 35 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPDDFATYYCQQSYRTPPTFGQGTKLEIK

SEQ ID NO: 36 - protein, 108 aa, synthetic construct
QSALTQDPAVSVALGQTVRITCQGDSLRSYYASWYQQKPGQAPVLVIYGKNNRPSGIPDRFSGSSSGNTASLTITGAQAEDEADYYCNSRDSSGNHWVFGGGTKLTVL

SEQ ID NO: 37 - protein, 110 aa, synthetic construct
QSELTQPPSASGAPGQRVTISCSGSSSNIGSNYVYWYQQLPGTAPKLLIYRNNQRPSGVPDRFSGSKSGTSASLAISGLRSEDEADYYCAAWDDSLSGPVFSGGTKLTVL

SEQ ID NO: 38 - protein, 13 aa, synthetic construct
  VARIANT 1: note = A or K
  VARIANT 2: note = A or T
  VARIANT 5: note = F, G, or F
  VARIANT 8: note = S or T
  VARIANT 9: note = S or N
  VARIANT 11: note = G, S, or A
  VARIANT 12: note = M or I
  VARIANT 13: note = H, N, or S
XXSGXTFXXYXXX

SEQ ID NO: 39 - protein, 10 aa, synthetic construct
  VARIANT 5: note = Y or N
  VARIANT 9: note = T or S
WISAXNGNXN

SEQ ID NO: 40 - protein, 11 aa, synthetic construct
  VARIANT 1: note = Q or A
  VARIANT 5: note = D or Y
  VARIANT 8: note = N or S
XASQXISXYLN

SEQ ID NO: 41 - protein, 8 aa, synthetic construct
  VARIANT 2: note = G or R
  VARIANT 3: note = K or N
  VARIANT 5: note = N or Q
YXXNXRPS

SEQ ID NO: 42 - protein, 9 aa, synthetic construct
  VARIANT 1: note = L or Q
  VARIANT 3: note = D or S
  VARIANT 5: note = N, S, or R
  VARIANT 6: note = Y or T
  VARIANT 8: note = L or P
XQXYXXPXT

SEQ ID NO: 43 - protein, 217 aa, unidentified
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 151 aa, unidentified
VINIVHKTLVPRPAVLDPALCTATSCVQVELCFAYNQSAGNPNYRRNITLAYTLEADRDRRPPRLRFAGSESAVFHGFFSMPEMRCQKLELLLMDNLRDKLRPIIISMNYSLPLRMPDRPRLGLRSLDAYPILNQAQALENHTEVQFQKEC

SEQ ID NO: 45 - protein, 1051 aa, unidentified
MGPGPSRAPRAPRLMLCALALMVAAGGCVVSAFNLDTRFLVVKEAGNPGSLFGYSVALHRQTERQQRYLLLAGAPRELAVPDGYTNRTGAVYLCPLTAHKDDCERMNITVKNDPGHHIIEDMWLGVTVASQGPAGRVLVCAHRYTQVLWSGSEDQRRMVGKCYVRGNDLELDSSDDWQTYHNEMCNSNTDYLETGMCQLGTSGGFTQNTVYFGAPGAYNWKGNSYMIQRKEWDLSEYSYKDPEDQGNLYIGYTMQVGSFILHPKNITIVTGAPRHRHMGAVFLLSQEAGGDLRRRQVLEGSQVGAYFGSAIALADLNNDGWQDLLVGAPYYFERKEEVGGAIYVFMNQAGTSFPAHPSLLLHGPSGSAFGLSVASIGDINQDGFQDIAVGAPFEGLGKVYIYHSSSKGLLRQPQQVIHGEKLGLPGLATFGYSLSGQMDVDENFYPDLLVGSLSDHIVLLRARPVINIVHKTLVPRPAVLDPALCTATSCVQVELCFAYNQSAGNPNYRRNITLAYTLEADRDRRPPRLRFAGSESAVFHGFFSMPEMRCQKLELLLMDNLRDKLRPIIISMNYSLPLRMPDRPRLGLRSLDAYPILNQAQALENHTEVQFQKECGPDNKCESNLQMRAAFVSEQQQKLSRLQYSRDVRKLLLSINVTNTRTSERSGEDAHEALLTLVVPPALLLSSVRPPGACQANETIFCELGNPFKRNQRMELLIAFEVIGVTLHTRDLQVQLQLSTSSHQDNLWPMILTLLVDYTLQTSLSMVNHRLQSFFGGTVMGESGMKTVEDVGSPLKYEFQVGPMGEGLVGLGTLVLGLEWPYEVSNGKWLLYPTEITVHGNGSWPCRPPGDLINPLNLTLSDPGDRPSSPQRRRRQLDPGGGQGPPPVTLAAAKKAKSETVLTCATGRAHCVWLECPIPDAPVVTNVTVKARVWNSTFIEDYRDFDRVRVNGWATLFLRTSIPTINMENKTTWFSVDIDSELVEELPAEIELWLVLVAVGAGLLLLGLIILLLWKCGFFKRARTRALYEAKRQKAEMKSQPSETERLTDDY

SEQ ID NO: 46 - protein, 798 aa, unidentified
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 47 - protein, 446 aa, synthetic construct
EVQLLESGGGVVQPGRSLRLSCAASGFTFSSYGMHWVRQAPGKGLEWLSGISGSADTTYYADSVKGRFTISRDNSKNTLYLQMTSLRAEDTAVYYCVRDDIQLRDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 214 aa, synthetic construct
DIQMTQSPSSLSASVGDRVTITCQASQDISNYLNWYQQKPGKAPKLLIYDASNLETGVPSRFSGSGSGTDFALTISSLQPEDFATYYCLQDYNYPLTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 455 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDYSGSWYPSNGPALDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 214 aa, synthetic construct
DIQMTQSPSSLSASVGDRVTITCRASQYISSYLNWYQQKPGKAPKLLIYDASNLETGVPSRFSGSGSGTDFTFTISSLQPEDIATYYCLQDYSYPLTFGGGIKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 448 aa, synthetic construct
EVQLVQSGAEVKKPGASVKVSCKTSGFTFTNYGISWVRQAPGQGLEWMGWISANNGNSNYAQDHQGRVTMTTDTSTSTAYMELRSLRSDDTAVYYCAREFPGWYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPDDFATYYCQQSYRTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 456 aa, synthetic construct
EVQLVESGGGLVQPGGSLRLSCAASGFTFSSYSMNWVRQAPGKGLEWVSSISSSSSYIYYADSVKGRFTISRDNSKNTVYLQMNSLRAEDTAVYYCAREYYDFWSGYPSGYAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 215 aa, synthetic construct
QSALTQDPAVSVALGQTVRITCQGDSLRSYYASWYQQKPGQAPVLVIYGKNNRPSGIPDRFSGSSSGNTASLTITGAQAEDEADYYCNSRDSSGNHWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 453 aa, synthetic construct
QVQLVQSGAEVKKPGSSVKVSCKASGGTFSSYAINWVRQAPGQGLEWMGGIIPIFGTANYAQKFQGRVTITADKSTSTAYMELSSLRSEDTAVYYCARGVPSGSGYYLGLDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 217 aa, synthetic construct
QSELTQPPSASGAPGQRVTISCSGSSSNIGSNYVYWYQQLPGTAPKLLIYRNNQRPSGVPDRFSGSKSGTSASLAISGLRSEDEADYYCAAWDDSLSGPVFSGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 19 aa, synthetic construct
MGWSCIILFLVATATGVHS

SEQ ID NO: 58 - protein, 9 aa, synthetic construct
MDYKDDDDK

SEQ ID NO: 59 - protein, 6 aa, synthetic construct
HHHHHH

SEQ ID NO: 60 - protein, 15 aa, synthetic construct
GGGGSGGGGSGGGGS

SEQ ID NO: 61 - protein, 8 aa, synthetic construct
LEVLFQGP

SEQ ID NO: 62 - protein, 1064 aa, synthetic construct
MGPGPSRAPRAPRLMLCALALMVAAGGCVVSAFNLDTRFLVVKEAGNPGSLFGYSVALHRQTERQQRYLLLAGAPRELAVPDGYTNRTGAVYLCPLTAHKDDCERMNITVKNDPGHHIIEDMWLGVTVASQGPAGRVLVCAHRYTQVLWSGSEDQRRMVGKCYVRGNDLELDSSDDWQTYHNEMCNSNTDYLETGMCQLGTSGGFTQNTVYFGAPGAYNWKGNSYMIQRKEWDLSEYSYKDPEDQGNLYIGYTMQVGSFILHPKNITIVTGAPRHRHMGAVFLLSQEAGGDLRRRQVLEGSQVGAYFGSAIALADLNNDGWQDLLVGAPYYFERKEEVGGAIYVFMNQAGTSFPAHPSLLLHGPSGSAFGLSVASIGDINQDGFQDIAVGAPFEGLGKVYIYHSSSKGLLRQPQQVIHGEKLGLPGLATFGYSLSGQMDVDENFYPDLLVGSLSDHIVLLRARPILHVSHEVSIAPRSIDLEQPNCAGGHSVCVDLRVCFSYIAVPSSYSPTVALDYVLDADTDRRLRGQVPRVTFLSRNLEEPKHQASGTVWLKHQHDRVCGDAMFQLQENVKDKLRAIVVTLSYSLQTPRLRRQAPGQGLPPVAPILNAHQPSTQRAEIHFLKQGCGPDNKCESNLQMRAAFVSEQQQKLSRLQYSRDVRKLLLSINVTNTRTSERSGEDAHEALLTLVVPPALLLSSVRPPGACQANETIFCELGNPFKRNQRMELLIAFEVIGVTLHTRDLQVQLQLSTSSHQDNLWPMILTLLVDYTLQTSLSMVNHRLQSFFGGTVMGESGMKTVEDVGSPLKYEFQVGPMGEGLVGLGTLVLGLEWPYEVSNGKWLLYPTEITVHGNGSWPCRPPGDLINPLNLTLSDPGDRPSSPQRRRRQLDPGGGQGPPPVTLAAAKKAKSETVLTCATGRAHCVWLECPIPDAPVVTNVTVKARVWNSTFIEDYRDFDRVRVNGWATLFLRTSIPTINMENKTTWFSVDIDSELVEELPAEIELWLVLVAVGAGLLLLGLIILLLWKCGFFKRARTRALYEAKRQKAEMKSQPSETERLTDDY

SEQ ID NO: 63 - protein, 1075 aa, synthetic construct
MGPGPSRAPRAPRLMLCALALMVAAGGCVVSAFNLDTRFLVVKEAGNPGSLFGYSVALHRQTERQQRYLLLAGAPRELAVPDGYTNRTGAVYLCPLTAHKDDCERMNITVKNDPGHHIIEDMWLGVTVASQGPAGRVLVCAHRYTQVLWSGSEDQRRMVGKCYVRGNDLELDSSDDWQTYHNEMCNSNTDYLETGMCQLGTSGGFTQNTVYFGAPGAYNWKGNSYMIQRKEWDLSEYSYKDPEDQGNLYIGYTMQVGSFILHPKNITIVTGAPRHRHMGAVFLLSQEAGGDLRRRQVLEGSQVGAYFGSAIALADLNNDGWQDLLVGAPYYFERKEEVGGAIYVFMNQAGTSFPAHPSLLLHGPSGSAFGLSVASIGDINQDGFQDIAVGAPFEGLGKVYIYHSSSKGLLRQPQQVIHGEKLGLPGLATFGYSLSGQMDVDENFYPDLLVGSLSDHIVLLRARPVINIVHKTLVPRPAVLDPALCTATSCVQVELCFAYNQSAGNPNYRRNITLAYTLEADRDRRPPRLRFAGSESAVFHGFFSMPEMRCQKLELLLMDNLRDKLRPIIISMNYSLPLRMPDRPRLGLRSLDAYPILNQAQALENHTEVQFQKECGPDNKCQSNLQLVRARFCTRVSDTEFQPLPMDVDGTTALFALSGQPVIGLELMVTNLPSDPAQPQADGDDAHEAQLLVMLPDSLHYSGVRALDPAEKPLCLSNENASHVECELGNPMKRGAQVTFYLILSTSGISIETTELEVELLLATISEQELHPVSARARVFIELLQTSLSMVNHRLQSFFGGTVMGESGMKTVEDVGSPLKYEFQVGPMGEGLVGLGTLVLGLEWPYEVSNGKWLLYPTEITVHGNGSWPCRPPGDLINPLNLTLSDPGDRPSSPQRRRRQLDPGGGQGPPPVTLAAAKKAKSETVLTCATGRAHCVWLECPIPDAPVVTNVTVKARVWNSTFIEDYRDFDRVRVNGWATLFLRTSIPTINMENKTTWFSVDIDSELVEELPAEIELWLVLVAVGAGLLLLGLIILLLWKCGFFKRARTRALYEAKRQKAEMKSQPSETERLTDDY

SEQ ID NO: 64 - protein, 1051 aa, synthetic construct
MGPGPSRAPRAPRLMLCALALMVAAGGCVVSAFNLDTRFLVVKEAGNPGSLFGYSVALHRQTERQQRYLLLAGAPRELAVPDGYTNRTGAVYLCPLTAHKDDCERMNITVKNDPGHHIIEDMWLGVTVASQGPAGRVLVCAHRYTQVLWSGSEDQRRMVGKCYVRGNDLELDSSDDWQTYHNEMCNSNTDYLETGMCQLGTSGGFTQNTVYFGAPGAYNWKGNSYMIQRKEWDLSEYSYKDPEDQGNLYIGYTMQVGSFILHPKNITIVTGAPRHRHMGAVFLLSQEAGGDLRRRQVLEGSQVGAYFGSAIALADLNNDGWQDLLVGAPYYFERKEEVGGAIYVFMNQAGTSFPAHPSLLLHGPSGSAFGLSVASIGDINQDGFQDIAVGAPFEGLGKVYIYHSSSKGLLRQPQQVIHGEKLGLPGLATFGYSLSGQMDVDENFYPDLLVGSLSDHIVLLRARPVINIVHKTLVPRPAVLDPALCTATSCVQVELCFAYNQSAGNPNYRRNITLAYTLEADRDRRPPRLRFAGSESAVFHGFFSMPEMRCQKLELLLMDNLRDKLRPIIISMNYSLPLRMPDRPRLGLRSLDAYPILNQAQALENHTEVQFQKECGPDNKCESNLQMRAAFVSEQQQKLSRLQYSRDVRKLLLSINVTNTRTSERSGEDAHEALLTLVVPPALLLSSVRPPGACQANETIFCELGNPFKRNQRMELLIAFEVIGVTLHTRDLQVQLQLSTSSHQDNLWPMILTLLVDYTPLSIAGMAIPQQLFFSGVVRGERAMQSERDVGSKVKYEVTVSNQGQSLRTLGSAFLNIMWPHEIANGKWLLYPMQVELEGGQGPGQKGLCSPRPNILHLDVDSRDRRRRELEPPEQQEPGERQEPSMSWWPVSSAEKKKNITLDCARGTANCVVFSCPLYSFDRAAVLHVWGRLWNSTFLEEYSAVKSLEVIVRANITVKSSIKNLMLRDASTVIPVMVYLDPMEELPAEIELWLVLVAVGAGLLLLGLIILLLWKCGFFKRARTRALYEAKRQKAEMKSQPSETERLTDDY

SEQ ID NO: 65 - protein, 1034 aa, synthetic construct
MGWSCIILFLVATATGVHSFNLDTRFLVVKEAGNPGSLFGYSVALHRQTERQQRYLLLAGAPRELAVPDGYTNRTGAVYLCPLTAHKDDCERMNITVKNDPGHHIIEDMWLGVTVASQGPAGRVLVCAHRYTQVLWSGSEDQRRMVGKCYVRGNDLELDSSDDWQTYHNEMCNSNTDYLETGMCQLGTSGGFTQNTVYFGAPGAYNWKGNSYMIQRKEWDLSEYSYKDPEDQGNLYIGYTMQVGSFILHPKNITIVTGAPRHRHMGAVFLLSQEAGGDLRRRQVLEGSQVGAYFGSAIALADLNNDGWQDLLVGAPYYFERKEEVGGAIYVFMNQAGTSFPAHPSLLLHGPSGSAFGLSVASIGDINQDGFQDIAVGAPFEGLGKVYIYHSSSKGLLRQPQQVIHGEKLGLPGLATFGYSLSGQMDVDENFYPDLLVGSLSDHIVLLRARPVINIVHKTLVPRPAVLDPALCTATSCVQVELCFAYNQSAGNPNYRRNITLAYTLEADRDRRPPRLRFAGSESAVFHGFFSMPEMRCQKLELLLMDNLRDKLRPIIISMNYSLPLRMPDRPRLGLRSLDAYPILNQAQALENHTEVQFQKECGPDNKCESNLQMRAAFVSEQQQKLSRLQYSRDVRKLLLSINVTNTRTSERSGEDAHEALLTLVVPPALLLSSVRPPGACQANETIFCELGNPFKRNQRMELLIAFEVIGVTLHTRDLQVQLQLSTSSHQDNLWPMILTLLVDYTLQTSLSMVNHRLQSFFGGTVMGESGMKTVEDVGSPLKYEFQVGPMGEGLVGLGTLVLGLEWPYEVSNGKWLLYPTEITVHGNGSWPCRPPGDLINPLNLTLSDPGDRPSSPQRRRRQLDPGGGQGPPPVTLAAAKKAKSETVLTCATGRAHCVWLECPIPDAPVVTNVTVKARVWNSTFIEDYRDFDRVRVNGWATLFLRTSIPTINMENKTTWFSVDIDSELVEELPAEIEGTGGLLEVLFQGPGENAQLEKELQALEKENAQLEWELQALEKELAQGGDYKDDDDK

SEQ ID NO: 66 - protein, 781 aa, synthetic construct
MGWSCIILFLVATATGVHSQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDDTSGLLEVLFQGPGKNAQLKKKLQALKKKNAQLKWKLQALKKKLAQGGHHHHHH

SEQ ID NO: 67 - protein, 392 aa, synthetic construct
ESNLQMRAAFVSEQQQKLSRLQYSRDVRKLLLSINVTNTRTSERSGEDAHEALLTLVVPPALLLSSVRPPGACQANETIFCELGNPFKRNQRMELLIAFEVIGVTLHTRDLQVQLQLSTSSHQDNLWPMILTLLVDYTLQTSLSMVNHRLQSFFGGTVMGESGMKTVEDVGSPLKYEFQVGPMGEGLVGLGTLVLGLEWPYEVSNGKWLLYPTEITVHGNGSWPCRPPGDLINPLNLTLSDPGDRPSSPQRRRRQLDPGGGQGPPPVTLAAAKKAKSETVLTCATGRAHCVWLECPIPDAPVVTNVTVKARVWNSTFIEDYRDFDRVRVNGWATLFLRTSIPTINMENKTTWFSVDIDSELVEELPAEIEGTGGLLEVLFQGPGENHHHHHH

SEQ ID NO: 68 - protein, 233 aa, synthetic construct
MGWSCIILFLVATATGVHSMDYKDDDDKGGGGSGGGGSGGGGSLEVLFQGPLRARPVINIVHKTLVPRPAVLDPALCTATSCVQVELCFAYNQSAGNPNYRRNITLAYTLEADRDRRPPRLRFAGSESAVFHGFFSMPEMRCQKLELLLMDNLRDKLRPIIISMNYSLPLRMPDRPRLGLRSLDAYPILNQAQALENHTEVQFQLEVLFQGPGGGGSGGGGSGGGGSHHHHHH

SEQ ID NO: 69 - protein, 1036 aa, synthetic construct
MGWSCIILFLVATATGVHSFNLDTRFLVVKEAVNPGSLFGYSVALHRQTERQQRYLLLAGAPRDLAVGDDYTNRTGAVYLCPLTAHKDDCERMDISEKSDPDHHIIEDMWLGVTVASQGPAGRVLVCAHRYTKVLWSGLEDQRRMVGKCYVRGNDLQLDPGDDWQTYHNEMCNSNTDYLQTGMCQLGTSGGFTQNTVYFGAPGAYNWKGNSYMIQRKDWDLSEYSYRGSEEQGNLYIGYTVQVGNAILHPTDIITVVTGAPRHQHMGAVFLLKQESGGDLQRKQVLKGTQVGAYFGSAIALADLNNDGWQDLLVGAPYYFERKEEVGGAVYVFMNQAGASFPDQPSLLLHGPSRSAFGISIASIGDINQDGFQDIAVGAPFEGLGKVYIYHSSSGGLLRQPQQIIHGEKLGLPGLATFGYSLSGKMDVDENLYPDLLVGSLSDHIVLLRARPVINILHRTLVARPAVLDPALCTATSCVQVELCFAYNQSAGNPNYRRNITLAYTLEADRDRRPPRLRFARSQSSVFHGFFSMPETHCQTLELLLMDNVRDKLRPIVIAMNYSLPLRMPDRLKLGLRSLDAYPVLNQAQAMENHTEVHFQKECGPDNKCDSNLQMRAAFLSEQLQPLSRLQYSRDTKKLFLSINVTNSPSSQRAGEDAHEALLTLEVPSALLLSSVRPSGTCQANNETILCELGNPFKRNQRMELLIAFEVIGVTLHTRDLPVLLQLSTSSHQDNLQPVLLTLQVDYTLQASLSLMNHRLQSFFGGTVMGEAAMKTAEDVGSPLKYEFQVSPVGDGLAALGTLVLGLEWPYEVTNGKWLLYPTEITIHSNGSWPCQPSGNLVNPLNLTLSDPGVTPLSPQRRRRQLDPGGDQSSPPVTLAAAKKAKSETVLTCSNGRARCVWLECPLPDTSNITNVTVKARVWNSTFIEDYKDFDRVRVDGWATLFLRTSIPTINMENKTTWFSVDIDSELVEELPAEIEGTGGLLEVLFQGPGENAQLEKELQALEKENAQLEWELQALEKELAQGGDYKDDDDK

SEQ ID NO: 70 - protein, 781 aa, synthetic construct
MGWSCIILFLVATATGVHSQTDKNRCLKANAKSCGECIQAGPNCGWCTNTTFLQEGMPTSARCDDLEALKKKGCQPSDIENPRGSQTIKKNKNVTNRSKGMAEKLRPEDITQIQPQQLLLKLRSGEPQKFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTDRGEFFNELVGQQRISGNLDSPEGGFDAIMQVAVCGSLIGWRNVTRLLVFSTDAGFHFAGDGKLGGIVLPNDGQCHLENNVYTMSHYYDYPSIAHLVQKLSENNIQTIFAVTEEFQPVYKELKNLIPKSAVGTLSGNSSNVIQLIIDAYNSLSSEVILENSKLPDGVTINYKSYCKNGVNGTGENGRKCSNISIGDEVQFEISITANKCPNKESETIKIKPLGFTEEVEVVLQFICKCNCQSHGIPASPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCRCRVCECYPNYTGSACDCSLDTGPCLASNGQICNGRGICECGACKCTDPKFQGPTCETCQTCLGVCAEHKECVQCRAFNKGEKKDTCAQECSHFNLTKVESREKLPQPVQVDPVTHCKEKDIDDCWFYFTYSVNGNNEAIVHVVETPDCPTGPDDTSGLLEVLFQGPGKNAQLKKKLQALKKKNAQLKWKLQALKKKLAQGGHHHHHH

SEQ ID NO: 71 - protein, 399 aa, synthetic construct
MGWSCIILFLVATATGVHSDSNLQMRAAFLSEQLQPLSRLQYSRDTKKLFLSINVTNSPSSQRAGEDAHEALLTLEVPSALLLSSVRPSGTCQANNETILCELGNPFKRNQRMELLIAFEVIGVTLHTRDLPVLLQLSTSSHQDNLQPVLLTLQVDYTLQASLSLMNHRLQSFFGGTVMGEAAMKTAEDVGSPLKYEFQVSPVGDGLAALGTLVLGLEWPYEVTNGKWLLYPTEITIHSNGSWPCQPSGNLVNPLNLTLSDPGVTPLSPQRRRRQLDPGGDQSSPPVTLAAAKKAKSETVLTCSNGRARCVWLECPLPDTSNITNVTVKARVWNSTFIEDYKDFDRVRVDGWATLFLRTSIPTINMENKTTWFSVDIDSELVEELPAEIEGENHHHHHH